(19) United States
(12) Patent Application Publication
(10) Pub. No.: US 2022/0008839 A1
(43) Pub. Date: Jan. 13, 2022

(54) SEMI-AQUEOUS METHOD FOR EXTRACTING A SUBSTANCE

(71) Applicants: David P. Jackson, Saugus, CA (US); Mackenzie A. Jackson, Saugus, CA (US); John J. Lee, Santa Clarita, CA (US)

(72) Inventors: David P. Jackson, Saugus, CA (US); Mackenzie A. Jackson, Saugus, CA (US); John J. Lee, Santa Clarita, CA (US)

(73) Assignee: Clean Imagineering LLC, Santa Clarita, CA (US)

(21) Appl. No.: 17/372,399

(22) Filed: Jul. 9, 2021

Related U.S. Application Data
(60) Provisional application No. 63/050,307, filed on Jul. 10, 2020.
(60) Provisional application No. 63/212,254, filed on Jun. 18, 2021.

(51) Int. Cl. B01D 11/02; A61K 36/185; A23L 33/105; C12H 3/00; A23L 2/52; G01N 11/00; G01N 1/40

(52) U.S. Cl. CPC B01D 11/0288; B01D 2011/007; A23L 33/105; C12H 3/00; A23L 2/52; B01D 11/0292; B01D 11/028; G01N 11/00; G01N 1/4055; A61K 2236/37; A61K 2236/333; A61K 2236/331; A61K 2236/53; A61K 2236/51; A23V 2002/00; A61K 36/185

(57) ABSTRACT

A semi-aqueous method for extracting a substance. The method involves combining a first liquid or solid substance containing an extract with a semi-aqueous solution containing a water-soluble or water-emulsifiable (WSWE) compound. Said WSWE compound selectively dissolves extract during a dense phase CO₂ expansion and salting-out process, which is simultaneously co-extracted using said dense phase CO₂, and desolvated to produce a CO₂ salted-out solvent mixture containing extract. Said CO₂ salted-out solvent mixture is treated using various secondary processes. The present invention is useful for producing extracts for use as additives in pharmaceuticals, nutraceuticals, cosmetics, beverages, or foods, and for quantitative analysis.

### PRIORITY CLAIM

This application claims the benefit of U.S. Provisional Patent Applications 63/050,307 (filed 10 Jul. 2020) and 63/212,254 (filed 18 Jun. 2021), which are incorporated by reference in entirety.

### BACKGROUND

### Field of Invention

An enormous variety of natural solid and liquid substances, also called natural products or biomaterials, contain extractable materials, also called natural extracts, biomaterial extracts or simply extracts. Biomaterial extracts are considered valuable for use in foods, pharmaceuticals, nutraceuticals, and cosmetics. The process of obtaining valuable extracts from biomaterials is called value extraction, or simply extraction. Natural solid substances include, for example, plants, vegetables, herbs, microbes, fungi, soils, and animal tissues. Natural liquid substances include, for example, alcoholic beverages, fermented broths, and fermented foods. Biomaterial extracts obtained from natural products include, for example, proteins, fats, dietary fibers, sugars, antioxidants, essential oils, flavors, colors, and naturally fermented substances such as CBD and ethanol. In another example, decarboxylated natural products such as Cannabis and Hemp are extracted to recover psychoactive and bioactive biomaterial extracts such as tetrahydrocannabinol (THC) and cannabidiol (CBD), respectively.

Conventionally, biomaterial extracts are obtained using solid-liquid extraction (SLE) or liquid-liquid extraction (LLE) processes composed of several unit operations such as pre-treatment of biomaterial (i.e., drying, grinding, or decarboxylation) and post-treatment of biomaterial extracts (i.e., filtration, concentration, purification, or fractionation). According to Chemat, F. et al., “Green Extraction of Natural Products. Origins, Current Status, and Future Challenges”, Trends in Analytical Chemistry 118 (2019) 248-263 (Chemat et al.), the most impactful unit operation is the extraction process, particularly when it is not optimized. Conventional extraction processes are often time and energy consuming, require the use of huge amounts of water or petroleum solvents harmful for the environment and workers, and generate a large quantity of waste. Moreover, the resulting biomaterial extract is not entirely clean or safe as it may contain residual solvents, contaminants from raw material, or denatured compounds due to drastic extraction conditions (i.e., high solvent temperatures and long extraction periods). In this regard, practitioners of biomaterial extractions implement process intensification techniques to obtain higher extraction efficiency and higher quality extract while reducing extraction time, number of unit operations, energy consumption, quantity of solvent in the process, environmental impact, economical costs, and quantity of waste generated. These imperatives are part of the so-called “Green Extraction” initiatives.

Further to this, Chemat et al. state that green extraction involves the development and design of extraction processes which reduce energy consumption, use alternative solvents and renewable natural products, and ensure safe and high-quality extracts. In this regard, green extraction processes employ process intensification techniques such as ultrasonics, microwaves, pulsed electric fields, heating, mixing, centrifugation, and employ alternative solvents and solvent-based processes including dense phase CO₂ (supercritical and liquid CO₂) and pressurized solvent extraction techniques, for example subcritical water extraction. Subcritical water is an extraction technique that uses liquid water as an extractant (extraction solvent) at temperatures typically above the atmospheric boiling point of water (100° C., 1 atm), but below the critical point of water (374° C., 218 atm). Subcritical water extraction (SWE) is also referred to in the prior art as pressurized hot water extraction (PHWE), superheated water extraction, pressurized liquid extraction (PLE), and accelerated solvent extraction (ASE), all with water as a solvent.

The literature is full of case studies clearly demonstrating that the implementation of green extraction initiatives, and particularly, effective process intensification techniques increase biomaterial extract yields, reduces extraction time, and reduces solvent consumption, all of which decreases energy usage and operational costs. For example, typical extraction and separation processes use large quantities of organic solvents. Although organic solvents (i.e., n-hexane) have well-known performance advantages, their replacement with greener alternatives is an imperative due to their toxic effects on the human health and the environment. Further to this, most conventional extraction solvents are classified as Volatile Organic Compounds (VOCs), hazardous air pollutants (HAPs), or greenhouse gases (GHGs), and increase the risks of fire and explosion.

Conventional so-called exhaustive extraction processes such as liquid-liquid and solid-liquid extraction typically utilize a significant volume of solvent (with or without co-solvent modifiers or additives), heat, and long processing times. For example, many large-capacity biomaterial extraction processes employ n-hexane, a highly flammable solvent known to be hazardous to humans and the ecosystem. In this regard, residual amounts of n-hexane invariably contaminate the extracts obtained using same. Moreover, nonpolar green solvents such as supercritical CO₂ and liquid CO₂ provide environmental protection and human safety, but produce a narrower extract range (i.e., exhibit extract selectivity) due to extraction-extract factors such as biomaterial morphology, molecular weight, chemical complexity, and polarity. Finally, eco-friendly and natural solvents such as fermented ethanol provide a much broader range of extract solubility but pose significant fire hazards if used in large volumes and leave the biomaterial saturated in ethanol following processing. Although a single solvent system is useful for obtaining a limited range of bulk extract from a substance, it is not efficient for full-spectrum extraction applications utilizing conventional extraction techniques, mainly attributed to physicochemical constraints and long processing times.

An alternative approach for resolving environmental, health, safety (EHS), and solvent selectivity constraints is to use blends of bio-based solvents, for example hydroethanolic solvent blends. However, although hydroethanolic solvent blends may address EHS constraints, these compounds introduce their own solvent selectivity and recyclability constraints. For example, hydroethanolic blends with ethanol:water ratios different from 95%:5% by volume is difficult to maintain and recycle due to evaporation and azeotropic distillation challenges, and hydroethanolic blends possessing ethanol content less than 80% by volume are much less selective for hydrocarbon-like extracts due to higher cohesion energy.

Biomaterial extracts possess solubility chemistries (i.e., molecular cohesion properties) that can range the entire Hansen solubility parameter spectrum, from hexane-like (14.9 MPa^{1/2}) to water-like (47.8 MPa^{1/2}), and include a wide range of molecular weights, polarities, molecular complexities, polar surface areas (P.S.A.), and hydrogen bonding properties. Moreover, biomaterial extracts obtained from plants, vegetables, and herbs are often located within physical structures such as highly polar and high molecular weight cutaneous or cellulosic membranes which require swelling (pore expansion) to improve solvent access and diffusion processes and require heat to improve swelling and extract solubility. As such, optimizing an exhaustive extraction process (i.e., maximizing extract yield in a minimal amount of time) requires (at a minimum) optimization of the following key extraction process variables:

(1) Mechanical energy inputs (i.e., solvent mixing and flow);
(2) Thermal energy inputs (i.e., high solvent temperature); and
(3) Chemical energy inputs (i.e., like-dissolves-like or like-swells-like).

In this regard, many conventional and newer green extraction techniques do not lend themselves to efficient full-spectrum extraction optimization due to several constraining factors including low boiling points, high volatility, high pressure implementation, temperature limitations, flammability, and limited cohesion energy in terms of polarity and hydrogen bonding, among others. For example, dense phase CO₂ solvent extraction processes are considered green extraction technology but cannot effectively implement acoustic (mechanical energy) and high solvent temperatures (thermal energy). Pure liquid carbon dioxide transitions from liquid phase to supercritical above 31° C., and supercritical carbon dioxide requires much higher pressures to maintain adequate solvent power (chemical energy) and extract (solute) carrying capacity as the CO₂ fluid temperature rises, all of which constrains this green extraction process; resulting in longer processing times and increased operational cost of the extraction process.

### Prior Art

There is a significant amount of prior art relevant to the present invention pertaining to the extraction and recovery of natural extracts from a biomaterial. The following discussion focuses on four most relevant prior art extraction technologies; dense phase gas extraction, organic solvent extraction, water-based extraction, and salting-out assisted liquid-liquid

TABLE 1
Properties of Dense Phase CO₂ (Compared to Fluorocarbon Solvents)

| | SOLVENT | | | | |
|---|---|---|---|---|---|
| | | | | HFE-7100 | |
| | | | | methoxy- | CFC-113 |
| | Carbon Dioxide | | | nonafluorobutane | trichlorotrifluoroethane |
| | (CO₂) | | | (C₄F₉OCH₃) | (Cl₂FC—CClF₂) |
| PROPERTY | Solid | Liquid | Supercritical | Liquid | Liquid |
| Density | 1.6 | 0.8 | 0.5 | 1.5 | 1.6 |
| g/ml | | | | | |
| Viscosity | — | 0.07 | 0.03 | 0.58 | 0.75 |
| mN-s/m² | | | | | |
| Surface | 5 | 5 | 0 | 19 | 19 |
| Tension | | | | | |
| mN/m | | | | | |
| Solubility | 22 | 17.9 | 14 | 13 | 15 |
| Parameter | | | | | |
| MPa^{1/2} | | | | | |
| KB Value | 20-30 | 20-30 | 10-20 | 10 | 14 |
| Conditions | −78° C., 1 | 20° C., 60 | 35° C.,100 | 25° C., 1 | 25° C., 1 |
| | atm | atm | atm | atm | atm |

extraction (SALLE).

### Dense Phase Gas Extraction Technology

In Hansen, C. M., Handbook of Solubility Parameters: A User's Handbook, 2^{nd} Edition, CRC Press, 2007 (Hansen), Hansen provides the experimentally derived solubility or cohesion chemistry of (nonpolar) dense phase CO₂ to be between 0 MPa^{1/2} (i.e., high pressure gas/vapor, a non-solvent) and 18 MPa^{1/2} (i.e., saturated liquid phase), and dependent upon both temperature and pressure conditions (Hansen, page 189, FIG. 10.3). Compared to liquid CO₂, supercritical CO₂ is a tunable (highly selective) solvent. The Hansen solubility or cohesion energy parameters are more dependent upon both the temperature and pressure conditions above its critical point (31° C. and 73 atm), or pseudocritical point as already discussed, due to high compressibility and non-condensing fluid properties. Relevant to the present invention, Hansen solvent cohesion properties useful for practicing the present invention are preferably close to those of the nonpolar and polar cannabinoids, terpenoids, and flavonoids to be extracted from cannabis-hemp and other natural organic compounds, as well as alcoholic beverages containing fermented and additive organic compounds. Chemical and physical properties of dense phase CO₂ (compared to fluorocarbon solvents) is provided under Table 1.

An example of prior art using liquid CO₂ to extract a biomaterial is U.S. Pat. No. 7,344,736 ('736), B. Whittle et al., “Extraction of Pharmaceutically Active Components from Plant Materials”. '736 teaches a method for extracting botanical compounds such as cannabidiol (CBD) from plant materials using liquid CO₂. The '736 method comprises first using a conventional thermal decarboxylation step to convert CBD-A (CBD acid form) contained in the botanical material to CBD, followed by selectively extracting the CBD from plant material using liquid CO₂. Following this, a final processing step is performed to reduce the proportion of non-target materials such as lipids and chlorophyll contained within the resulting CO₂ extract. The last step is partly accomplished by performing the extraction step using CO₂ under subcritical (liquid) conditions at a temperature in the range from 5° C. to 15° C. at a pressure between 50 atm and 70 atm, which is cooler than conventional supercritical CO₂ extraction processes operating well above 31.1° C. and pressures greater than 73 atm, and but not as cool as conventional cold organic solvent extraction methods operating at atmospheric pressure.

Much of the prior art involving dense phase CO₂ processes is focused on selectively removing or isolating compounds from a plant material, for example isolating CBD from chlorophyll during extraction. More recently, full-spectrum extraction is becoming recognized as a more attractive and complete method for producing healthful or beneficial botanical extracts. A full-spectrum extract is an extraction of all the plants beneficial and natural compounds—for example cannabinoids, terpenes, and flavonoids together. Preferably, an alcohol such as ethanol is recommended and is derived from any number of commercial processes. CBD is just one part of the whole cannabinoid spectrum. This spectrum is where the plant holds its synergy with the endocannabinoid system within the body. Any modifications to the natural spectrum of cannabinoids will degrade the synergy that nature intended the plant to have. When a high-CBD content hemp plant is extracted via supercritical or liquid CO₂, mostly the cannabinoids are extracted leaving behind most of the terpenoids. This makes for a purer extract, but lacks the full-spectrum of extractables the plant has to offer. Both propane and butane extractions are also very selective in this regard and may also leave behind various chemical residues or impurities contained in these flammable dense fluids. According to Russo, E. B., “Taming THC: potential cannabis synergy and phytocannabinoid-terpenoid entourage effects”, British Journal of Pharmacology (2011) 163 1344-1364 (Russo), it is becoming more apparent from pharmacological studies that a full-spectrum of hemp or cannabis compounds are much more beneficial (i.e., due to the so-called “entourage effect”), as cannabinoids alone do not have the highest medicinal benefits as compared to a mixture of terpenes, cannabinoids, and other synergistic compounds. As such, a full-spectrum solvent system that can extract a full-spectrum of botanical compounds is most desirable. Solvent selectivity is necessary for isolating a certain class or group of organic compounds, for example for a medical or food use. For example, supercritical CO₂ is selective for CBD, however a portion of volatile terpenes are lost during CBD oil recovery operations (i.e., depressurization). Liquid CO₂ is less selective than supercritical CO₂ in this regard, and ethanol even less selective than both CO₂ solvent extraction methods.

More elaborate dense phase CO₂ separation methods such as the CO₂ solvent phase-shifting process are taught by the first-named inventor of the present invention and described in U.S. Pat. No. 5,013,366 ('366), D. P. Jackson, “Cleaning Process using Phase Shifting of Dense Phase Gases”. The '366 phase shifting process is adaptable to botanical extractions (i.e., replace hardware processing with plant processing) to provide a much broader range of extractables, and particularly with the addition of polar co-solvent additives such as ethanol, IPA, and acetone. '366 teaches a novel phase shifting process to extract compounds from a substrate by shifting the cohesion energy of a dense fluid extraction solvent between a subcritical (liquid) phase and supercritical state. Although directed particularly to cleaning hardware used in high vacuum or space-borne system applications, '366 is easily adaptable to and useful for the present field of invention and application. However, it should be noted that the '366 CO₂ phase shifting process, as well as most any large-scale production dense phase gas extraction process, is a very time consuming and costly method (i.e., capital equipment) for producing a full-spectrum extract.

Another exemplary dense phase CO₂ process developed by the first named inventor of the present invention is a hybrid extraction process, detailed in U.S. Pat. No. 7,601,112 ('112), D. P. Jackson, “Dense Fluid Cleaning Centrifugal Phase Shifting Separation Process and Apparatus”. Like '366, the '112 process was developed for and directed at precision cleaning (extraction) of manufactured articles used in critical applications. However, identical to the '366 process, the '112 process is directly (without modification) suitable for use in any botanical extraction application. For example, a porous or semi-permeable basket or cellulosic bag, and which is chemically and physically compatible with the CO₂ solvent system and process, can be used to contain a botanical material (wet, dry, particulate or whole) and processed in accordance with the process described in '112. The '112 process employs a bi-directional and/or tumbling centrifugal separation apparatus in cooperation with one or more organic solvent pre-wash operations (pre-extraction step), followed by a liquid or supercritical CO₂ rinsing (post-extraction process agent). Moreover, CO₂ is used as a compressed gas solute to enhance the performance of the organic solvent pre-wash step, providing lower surface tension, lower viscosity, and froth flotation effects. As applied to botanical material extractions, the broad mechanical and chemical processing capability of '112 ensures fast, efficient, and complete full-spectrum botanical extractions. However, although the '112 process is much more efficient than '366 for producing a full-spectrum extract, the '112 process also suffers from drawbacks such as a very high capital cost and system complexity and employs relatively high temperature and pressure conditions.

Related to the dynamic centrifugal and solvent prewash processes described in '112, another example of a mechanical (process intensification) method used in cooperation with an organic solvent for botanical extraction is described in U.S. Pat. No. 2,680,754 ('754), J. J. Liewenald, “Solvent Extraction of Oils, Fats, and Waxes from Particles of Solid Matter”. '754 teaches a centrifuge-based nonpolar liquid hexane solvent extraction process for removing and refining extractable oils from both plant and animal products.

The present invention utilizes CO₂ in different phases and capacities. As such, a particularly important aspect of CO₂-based or CO₂-assisted extraction processes is the recycling and purification of CO₂, and particularly in high-production extraction applications. In this regard, U.S. Pat. No. 6,979,362 ('362), D. P. Jackson, “Apparatus and Process for the Treatment, Delivery, and Recycle of Process Fluids used in Dense Phase Carbon Dioxide Applications”, developed by the first-named inventor of the present invention, describes a novel low-energy isobaric CO₂ recycling process. The '362 invention is easily adapted to a CO₂-based or CO₂-assisted biomaterial extraction process to provide a simple desolvation and extract recovery capability.

Finally, dense phase gases other than CO₂ are used in botanical material extractions. For example, the so-called Butane Honey Oil (BHO) extraction method employs liquid butane, a highly flammable dense phase gas solvent, at relatively low pressure and temperature to make a cannabis “red oil” commonly called hash oil. Liquid propane is similarly used in this regard, termed Propane Honey Oil (PHO) extraction. Like dense phase CO₂ extractions, liquid butane or propane are used to extract CBD and THC from cannabis and separated in a phase change process to recover both the dense fluid and extract. One advantage of using butane or propane compared to dense phase CO₂ is much lower operating pressures, but a major drawback is the fire and explosion hazards associated with using flammable dense fluids in extraction processes. Both flammable dense fluids can extract a high percentage of botanical compounds such as cannabinoids, terpenes, and flavonoids. However, because of nonpolar cohesion energy properties, butane and propane also extract relatively nonpolar hydrophobic constituents such as plant waxes and lipids.

Having discussed various and exemplary prior art dense phase gas solvent extraction techniques, following is a discussion of liquid (organic) solvent extraction techniques.

### Organic Solvent Extraction Technology

Liquid organic solvents and solvent blends (non-aqueous and semi-aqueous) used to extract biomaterials are numerous and include so-called green or naturally derived solvents. Examples of non-aqueous extraction solvents include ethanol, isopropyl alcohol (IPA), hexane, and acetone. These solvents can be used at extremely low extraction temperatures due to their low melt points and as relatively hot solvents in Soxhlet extraction processes. The main advantages of green non-aqueous solvents are their low toxicity, low cost and relatively low boiling points relative to botanical extracts such as terpenes, cannabinoids, and flavonoids which facilitates easy separation and recovery of both solvent and extract by simple distillation separation means, including vacuum- and heat-assisted distillation techniques. New separation methods include nanofiltration using special membranes under pressure to create solvent- and extract-rich phases. The main disadvantage of many non-aqueous organic solvents useful for botanical extractions is their inherent flammability (and low flash point temperatures), which requires specially designed equipment and facilities for safe handling and operation, particularly when used in large volume. In this regard, textbook resource, Smallwood, I., Solvent Recovery Handbook, McGraw-Hill, Inc., 1993 (Smallwood), Smallwood provides a detailed description of liquid organic solvent physicochemistry, treatment and recovery methods, and health, safety, and regulatory aspects related to the use, recovery, and management of organic solvents commonly used in liquid organic solvent extraction processes.

There are numerous methods and processes to extract compounds from biomaterials using non-aqueous solvents. The non-aqueous solvents may be miscible with water (i.e., acetone, IPA, ethanol) or water-immiscible (i.e., Hexane) and vary in solvent power—in terms of Kauri-Butanol (KB) value, cohesion parameter (Hansen Solubility Parameter (HSP)), and polarity. An example of a nonpolar solvent extraction process is described in U.S. Pat. No. 6,365,416 ('416), M. A. Elsohly, “Method for Preparing Delta-9-TetraHydroCannabinol”. The '416 process uses a nonpolar solvent such as hexane to selectively extract predominantly nonpolar extracts followed by vacuum distillation and chromatography to separate (purify) the THC compound from the full-spectrum of lipids, terpenes, chlorophyll, waxes and the like.

Semi-aqueous solvent blends, so-called hydroethanolic solvents, comprising ethanol and water are used to extract biomaterials. In U.S. Ser. No. 14/711,030, US 2016/02228787 ('787), J. F. Payack, “Method and Apparatus for Extracting Plant Oils using Ethanol Water”, a Soxhlet type extraction method is used to continuously provide fresh Ethanol-Water azeotrope mixture (95%:5% v:v) to a plant material during extraction. The main benefit of the '787 process is that a relatively dry and pure azeotropic ethanol solvent is continuously presented to the botanical material which continuously drives solute concentration-driven extraction phenomenon in accordance with Fick's Law. However, drawbacks of this process are that the solvent is heated to its boiling point (78.1° C.) which will solubilize most non-volatile botanical compounds including undesirable chlorophyllins, waxes, and lipids, and potentially volatilize beneficial terpenoids.

In Jacotet-Navarro, M. et al., “What is the best ethanol-water ratio for the extraction of antioxidants from rosemary? Impact of the solvent on yield, composition, and activity of the extracts”, Electrophoresis 2018, 0, 1-11 (Jacotet-Navarro et al.), Jacotet-Navarro et al. state that botanical compounds such as CBD and THC located in the trichomes of the leaves (i.e., hemp, cannabis) are easily accessible for extraction using an organic solvent such as ethanol. By contrast, botanical compounds located in more complex cellulosic plant structures requires enhanced mass transfer of the solvent to improve extraction efficiency. A critical aspect of a natural product extraction process is the proper selection of solvent and extraction conditions to provide maximum efficiency. Solubilization is not the only process that drives the extraction process. Other important phenomena must be taken into consideration. According to Jacotet-Navarro et al., “plant extraction” is more accurately depicted as a sequential molecular mass and energy transfer process, roughly divided into three steps:
(i) diffusion of the solvent through the plant material core;
(ii) desorption of the targeted compound from the plant matrix due to chemical affinity with the solvent; and
(iii) mass transfer of the solute from the plant vicinity to the solvent bulk.

For example, polyphenols accumulate in the vacuole of plant cells located within the interior plant anatomy in rosemary leaves. As such, an extraction solvent must cross several cell compartments such as cellulosic walls and membranes to get to the vacuole. Increasing water content improves mass transfer (extraction efficiency) of ethanol into these plant structures, likely due to the need to have a higher cohesion energy to swell the cellulosic plant walls and to dissolve and remove water-soluble and amphiphilic phospholipids (plasticizer) from the polymeric membranes, and which softens same for better water-ethanol solvent penetration. Moreover, water-ethanol mixtures have a cohesion energy chemistry that better matches the solubility chemistry of the target polyphenol compounds. As such, controlling the chemical and physical properties of an extraction solvent can have a huge impact on solvent extraction efficiency. Further to this, in Yamamoto, H. et al., “Separation of Polyphenols in Hop Bract part discharged from Beer Breweries and their Separability Evaluation Using Solubility Parameters”, KAGAKU KOGAKU RONBUNSHU, The Society of Chemical Engineers, Japan, Volume 34 (2008) Issue 3 (Yamamoto et al.), Yamamoto et al. determined the maximum efficiency for polyphenol extraction from Hop bract using a 50:50 (by weight) solution of ethanol and water, which has an approximate (and calculated) solubility parameter of 37 MPa^{1/2}. As such and relevant to the present invention, it is understood by those skilled in the art that both optimal mass diffusion properties and solubility parameters are necessary to achieve maximum extraction efficiency.

Moreover, several relatively new solvent-based extraction processes are being used to extract and recover CBD and THC from hemp and cannabis. One such technique employs liquid nitrogen injection to super cool a low melt-point organic solvent prior to and during a botanical material extraction process. Like the use of an external refrigeration process, direct cooling with liquid nitrogen injection imparts no beneficial co-solvency effects as nitrogen gas exhibits no solvent solubility and induces no beneficial changes in organic solvent properties such as expansion, regardless of temperature and pressure used.

Finally, another relatively new non-aqueous solvent extraction technique to be discussed, and relevant to the present invention, combines a compressed CO₂ gas and a heated liquid organic solvent under elevated pressure and temperature—termed gas-expanded liquid extraction or simply “GXLE”. In Jessop, P. G. and Subramaniam, B., “Gas-Expanded Liquids”, Chem. Rev. 2007, 107, 2666-2694 (Jessop and Subramaniam), Jessop and Subramaniam detail the principles, practices, and applications using gas-expanded liquids using compressed gases such as ethane and CO₂. Jessop and Subramaniam note that several research groups have clearly demonstrated how these relatively new solvents, called gas-expanded liquids (GXLs), are promising alternative media for performing separations, extractions, reactions, and other applications. A GXL is a mixed solvent composed of a compressible gas (such as CO₂ or ethane) dissolved as a solute in a liquid organic solvent. Because of the safety and economic advantages of CO₂, CO₂-expanded liquids (CXLs) are the most used class of GXLs. By varying the CO₂ composition, a continuum of liquid media ranging from the neat organic solvent to supercritical CO₂ is generated, the properties of which can be adjusted by tuning the operating pressure; for example, a large amount of CO₂ favors mass transfer and, in many cases, gas solubility, and the presence of polar organic solvents enhances the solubility of solid and liquid solutes. CXLs have been shown to be optimal solvents in a variety of roles including inducing separations, precipitating fine particles, polymer processing, and serving as reaction media for catalytic reactions. Process advantages include ease of removal of the CO₂, enhanced solubility of reagent gases (compared to liquid solvents), fire suppression capability of the CO₂, and milder process pressures (tens of atmospheres) compared to supercritical CO₂ (hundreds of atmospheres). Environmental advantages include substantial replacement of organic solvents with environmentally benign dense phase CO₂. Thus, CXLs have emerged as important components in the optimization of chemical processes, for example botanical extractions.

As CO₂ dissolves into an organic liquid, the liquid expands volumetrically, forming a GXL. Not all liquids expand equally in the presence of CO₂ pressure, and the differences in expansion behavior are attributed to differences in the ability of CO₂ to dissolve into a liquid organic solvent. Analogous to “like-dissolves-like” and “like-seeks-like” general solubility rules, the smaller the differences between the cohesion energies (dispersion, hydrogen bonding, and polar solubility parameters) of the liquid solvent and CO₂ solute, the larger the solvent expansion effect. Regarding properties of CO₂ gas expanded liquids, dissolving compressed CO₂ into a liquid organic solvent decreases its dielectric permittivity and subsequently its polarizability as well as its solubility parameters. Furthermore, dissolving compressed CO₂ in a liquid organic solvent decreases its surface tension and viscosity, and thereby improves its mass transfer properties.

Another interesting phenomenon associated with CXL technology, and particularly CO₂ expanded liquid mixtures is miscibility changes. As discussed in the prior under Jessop and Subramaniam, when CO₂ is compressed into an organic solvent mixture comprising, for example, an alcoholic water solution at 40° C., the mixture can be split into two phases at a so-called (and specific) lower critical solution pressure (LCSP) to form a multiphasic solution comprising a water-rich phase, an alcohol-rich phase, and a CO₂ vapor phase. Moreover, a specific upper critical solution pressure (UCSP), which is essentially the formation of a supercritical CO₂ phase, is required to form a biphasic system comprising supercritical CO₂-alcohol phase and water-rich phase. The comprehensive prior art review of Jessop and Subramaniam focuses on CXL technology and phase behavior operating at elevated temperatures and pressures, for example 40° C. and 80 atm, typical of supercritical fluid processing technology. The comprehensive literature review and references of Jessop and Subramaniam, as well as other prior art disclosed herein, do not suggest exploiting CO₂ expansion and salting-out phenomenon in a novel method for extracting natural or environmental substances containing extractable substances such as essential oils or pollutants, respectively. Still moreover, heretofore, no known prior art has been discovered by the present inventors which describes temperature- and pressure-selective CO₂ expansion and salting-out solvent miscibility behaviors at subcritical dense phase CO₂ temperatures and pressures observed employing a (purposely) added water-miscible or water-emulsifiable (WSWE) compound (i.e., used as a primary extractant) in a solid-liquid or liquid-liquid semi-aqueous extraction solvent system and co-extracted by dense phase CO₂ (i.e., used as a secondary extractant and extract concentration solvent), as disclosed in the present invention. Further to this, the present inventors believe the selective phase separation behavior disclosed in the present invention is a unique characteristic of subcritical CO₂ expansion and salting-out phenomenon. In this regard, utilizing a semi-aqueous extraction solvent system in combination with subcritical CO₂ (gas-liquid) temperature and pressure conditions provides much higher aqueous CO₂ concentrations, which may explain selective salting-out behavior at pressures as low as 7 atm at a temperature of 20° C., disclosed herein. These distinctions are not disclosed in the prior art. As such, the synergistic combination of CO₂ expansion and salting-out phenomenon, collectively referred to herein as CO₂ salting-out, are uniquely exploited in the present invention as a biphasic or multiphasic solid-liquid or liquid-liquid extraction and extract concentration and recovery process called CO₂ Salting-out Assisted Liquid-Liquid Extraction (CO₂ SALLE) process.

In Al-Hamimi, S. et al., “Carbon Dioxide Expanded Ethanol Extraction: Solubility and Extraction Kinetics of α-Pinene and cis-Verbenol”, Anal. Chem. 2016, 88, 4336-4345 (Al-Hamimi et al.), Al-Hamimi et al. detail an experimental study of extraction kinetics during the extraction of medium-polar α-Pinene and cis-Verbenol (terpenes) from Boswellia sacra tree resin using a CXL process employing ethanol at a temperature of 40° C. and CO₂ at a pressure of 95 atm. As shown in Table 2, Al-Hamimi et al. calculated the solubility parameter for the CO₂-expanded liquid ethanol (CXLE) as 14.9 MPa^{1/2}, which is 42% lower than the solubility parameter of 25.8 MPa^{1/2} for pure ethanol at 1 atm and 40° C. Further to this, Al-Hamimi et al. showed that CO₂-expanded ethanol (CXE) is a high-diffusion extraction phase that provides fast and efficient extraction of medium polar compounds from a solid complex botanical material. Finally, Al-Hamimi et al. showed that CXLE is faster and more efficient than both supercritical CO₂ extraction (SFE, scCO₂) using an ethanol solute additive and conventional solvent liquid extraction (SLE) using pure ethanol. For example, the cis-verbenol extraction rate using CXLE was 10-fold faster than SFE.

TABLE 2
Solubility Parameter of CO₂-Expanded
Liquid Ethanol (95 atm and 40° C.)

| Solvent | δ_{T} - MPa^{1/2} | Conditions |
|---|---|---|
| EtOH | 25.8 | 40° C./1 atm |
| CXLE | 14.9 | 40° C./95 atm |
| Reduction: | 42% | |

CXLE - CO₂ Expanded Liquid Ethanol

Conventional GXLE processes typically employ CO₂ as a high-pressure gas or liquid which is injected into a liquid organic solvent having a temperature that is well above the critical temperature of CO₂. Adjusting the CO₂ concentration (using CO₂ gas pressure) within the solvent under these conditions provides a range of solvent cohesion energies ranging from (heated) neat liquid organic solvent to supercritical CO₂.

### Water-Based Extraction Technology

Water (H₂O) is a polar, colorless, and odorless inorganic compound often described as the “universal solvent”. It is the most abundant substance on the surface of Earth. Water molecules form hydrogen bonds with each other and are strongly polar. This strong polarity allows water to readily dissolve and dissociate salts and dissolve other polar substances such as alcohols and acids. Strong hydrogen bonding properties results in a moderately high boiling point (100° C.) and extremely high heat capacity. Finally, water is an amphoteric solvent, meaning that it can exhibit properties of an acid or a base, depending on the pH of the solution, and readily produces both H⁺ and OH⁻ ions.

In Plaza, M. et al., “Pressurized Hot Water Extraction of Bioactives”, Trends in Analytical Chemistry 71 (2015) 39-54 (Plaza et al.), the properties and benefits of using subcritical water in botanical extraction processes is detailed. When water is heated and pressurized to form a subcritical fluid, its dielectric and Hansen Solubility Parameter properties plummet, reaching levels like liquid organic solvents such as methanol and ethanol at room temperature. Moreover, adding organic substances to water, for example ethanol, surfactants, modifiers enhance the recovery of polyphenols from plant materials during pressurized heated water extraction. Moreover, the pH of water decreases with increasing temperature (and autogenous vapor pressure), lowering from a pH=7 at 25° C. to a pH=5.5 at 250° C. Still moreover, mass transfer properties of water improve significantly with increasing temperature. As temperature increases, viscosity decreases, diffusivity increases, and surface tension decreases to levels like or even less than conventional organic solvents—all of which improves mass transfer of extracts into subcritical water during extraction. Given this, subcritical water is potentially a universal and green extraction solvent for polar, nonpolar, mineral-based, and ionic extracts, and over a broad temperature (and pressure) spectrum.

In Mihaylova, D. et al., “Water an Eco-Friendly Crossroad in Green Extraction: An Overview”, The Open Biotechnology Journal, 2019, Volume 13, pp. 155-162 (Mihaylova et al.), numerous water-based extraction processes used to extract phytochemicals from biomaterials are contrasted and compared. Although water is considered a green and non-toxic extraction solvent for many different extraction techniques, a significant drawback is that a lot of energy is required to concentrate and recover dissolved extracts once removed from a solid substance. Using water as a solvent has nearly negligible environmental impact considering production and transportation. Exemplary water-based extraction methods include Soxhlet extraction, maceration, percolation, decoction, infusion, steam distillation, and heated pressurized water extraction or subcritical water extraction.

A foundational patent in subcritical water extraction is U.S. Pat. No. 7,943,190 ('190), G. Mazza and J. Eduardo Cacace, “Extraction of Phytochemicals”. '190 teaches various processing systems and methods for extracting phytochemicals from plant materials with subcritical water. The processing system includes a water supply interconnected with a high-pressure pump, diverter valve, a temperature-controllable extraction vessel, a cooler, a pressure-relief valve, and a collection apparatus for collecting eluent fractions from the extraction vessel. The processing system controllably varies the temperature of subcritical water within the extraction vessel and may optionally be configured to controllably vary the pH of subcritical water flowing into the extraction vessel. A plant material is placed into the extraction vessel after which a flow of subcritical water is provided through the extraction vessel for extraction of phytochemicals. The temperature of subcritical water is controllably varied between 55° C. and 373° C. during its flow through the extraction vessel water thereby producing a plurality of eluent sub-fractions corresponding to the temperature changes, thereby separating the different classes of phytochemicals extracted from the plant material. The high-pressure pump is used to pressurize said subcritical water at elevated temperatures to maintain a liquid state within the extractor.

Moreover, a multi-staged subcritical water extraction apparatus is detailed in U.S. Pat. No. 9,084,948 ('948), G. Mazza and C. Pronyk, “Pressurized Low Polarity Water Extraction Apparatus and Methods of Use”. '948 describes various multi-stage subcritical water extraction system designs for extraction and recovery of components from biomass feedstocks with pressurized low polarity water. The apparatus is configured with two or more reaction columns, each separately communicating with sources of pressurized water, pressurized heated water, and pressurized cooling water. Components are extracted from the biomass by separately flooding the column with pressurized water (using a mechanical high-pressure pump), heating the column and its contents to the point where the water becomes pressurized low polarity (PLP) water, recovering the PLP water comprising the extracted components, cooling the column with PLP water, and removing the spent biomass material from the column.

U.S. Pat. Nos. '190 and '948 do not prescribe a particular phytochemical extract concentration and recovery method for the water-based extractants produced or used by these inventions. Based on prior art discussed herein, the subcritical water solvent may be reused and concentrated with phytochemical extracts, evaporated to recover said extracts, or used directly as a water-based phytochemical additive concentrate in a formulation. Alternatively, a conventional extract concentration and recovery technique such as solid-phase extraction may be employed, followed by organic solvent extraction of solid-phase separation media, distillation of the organic solvent, and recovery of both the organic solvent and phytochemical extracts.

The main disadvantage of using water as a solvent in biomaterial extraction is the difficulty in concentrating the aqueous extracts, since the heat of vaporization of water is relatively high compared to that of many organic solvents. Furthermore, the need to concentrate the sample is often relevant, since the concentration of bioactive compound in the water extract could be extremely low. Using water as a solvent is energy intensive in applications where water needs to be removed to concentrate the extracts. Plaza et al. note that the energy demand to heat liquid water (25° C. to 250° C., 5 MPa) for extraction applications is almost three times less than needed to vaporize water to create steam (25° C. to 250° C., 0.1 MPa). As such, secondary (and often toxic) water-insoluble organic solvents such as hexane or methylene chloride are employed in a liquid-liquid extraction, desolvation, and extract recovery process.

Extract concentration and recovery methods such as freeze drying have been developed, but many are highly energy- and time-intensive. Plaza et al. describes a newer and more promising method called water extraction and particle formation on-line (WEPO). The WEPO method is a rapid expansion of supercritical solutions (RESS) micronization process that combines subcritical water solvent containing dissolved extracts with supercritical CO₂, which is rapidly expanded into a chamber to form dried particles comprising the extracts. The WEPO extract concentration and recovery method operates on the principle that subcritical water at high temperature (200° C.) and autogenous pressures (8 MPa, 80 atm, 1200 psi) is alcohol-like and can be solubilized with supercritical CO₂. However, due to the high heat capacity of water and the large Joule-Thomson expansion cooling effects during CO₂ expansion, RESS micronization processes involving water-based extraction solvents are much slower and less efficient than those using an organic solvent-extract system. Moreover, it is uneconomical to capture, recompress, and reuse the significant volume of carbon dioxide needed in a large-capacity RESS concentration and recovery operation using water-based extraction solvents. Finally, the main disadvantage of the WEPO process is high CO₂ consumption and, in general, aqueous-based RESS processes suffer from the operational and scale-up problems related with nozzle plugging due to accumulation of the particles (i.e., salts and extracts) in the fluid nozzle. To minimize this constraint, demineralized water is required as a base aqueous extractant fluid to lower salt content, and more dilute concentrations of subcritical water-extract are processed using the WEPO method, all of which adds additional processing time, extraction system complexity, and operating cost.

In summary, the main drawbacks of conventional water-based extraction are the high temperatures and time involved in extraction and concentration and recovery of extracts. As such, newer water-based extraction methods providing lower operating temperatures, faster processing (including extract recovery), and complete material input recovery are desirable.

Having thus described water-based extraction technologies relevant to the present invention, following is a discussion of a novel technique for extracting and recovering a dissolved analyte from an aqueous liquid, called salting-out assisted liquid-liquid extraction, or more simply “SALLE”.

### Salting-Out Assisted Liquid-Liquid Extraction (SALLE) Technology

The present invention discloses a novel extraction, co-extraction, and infusion process called CO₂ salting-out assisted liquid-liquid extraction (“CO₂ SALLE”), initially developed by the present inventors for salting-out and selectively extracting naturally fermented ethanol and dissolved ethanol-soluble and/or CO₂-soluble fermented compounds from a fermented liquid or broth for recovery or for co-extraction of a biomaterial. Said ethanol-soluble and/or CO₂-soluble compounds may be used to facilitate and infuse a biomaterial extract during a solid-liquid extraction process, for example hemp extraction. The CO₂ SALLE method and apparatus of the present invention is related to conventional salting-out assisted liquid-liquid extraction, or simply “SALLE”. SALLE is a popular laboratory sample preparation technique that uses a water-miscible organic solvent (e.g., acetone, acetonitrile, etc.) as an extraction solvent and one or more dissolved salts as phase separation (salting-out) agents. Briefly, following the addition of a water-soluble extraction solvent, significant amounts of one or more water-soluble salts (NaCl, K₂SO₄, K₂CO₃, etc.) are added to the aqueous solvent mixture to complex with the water molecules, which induces a phase separation of the water-miscible organic solvent. Interestingly, and relevant to the present invention, carbonate salts are shown to be one of the most effective salting-out agents. This aspect is discussed in more detail herein. The salted-out extraction solvent eventually forms a distinct upper (or lower) liquid phase, dependent upon the difference in density between the water-soluble organic solvent (salted-out solvent) and the (salted) aqueous solution. The salted-out liquid phase extracts (based on partition coefficients) a substantial fraction of the dissolved extracts (solutes) from the (salted) aqueous solution using turbulent salting-out mixing and phase separation using air flotation and centrifugal force. Compared to other laboratory sample preparation procedures, SALLE techniques are cheaper, faster, and considerably simpler to implement for small volume samples. Moreover, conventional SALLE techniques lend itself to direct coupling of the separated organic solvent layer containing dissolved extracts with a sample analytical technique such as high-performance liquid chromatography (HPLC). For example, in Alemayehu, Y. et al., “Salting-Out Assisted Liquid-Liquid Extraction Combined with HPLC for Quantitative Extraction of Trace Multiclass Pesticide Residues from Environmental Waters”, American Journal of Analytical Chemistry, 2017, 8, 433-448 (Alemayehu et al), Alemayehu et al. developed a SALLE technique combined with high performance liquid chromatography diode array detector (SALLE-HPLC-DAD) method for simultaneous analysis of carbaryl, atrazine, propazine, chlorothalonil, dimethametryn and terbutryn in environmental water samples.

However conventional SALLE techniques as developed by Alemayehu et al. are most efficient and cost-effective for only small-volume sample workups needed for laboratory analysis. Conventional SALLE techniques are difficult to adapt to larger capacity applications due to operating complexity and expense associated with using large amounts of highly flammable organic extraction solvents, one or more mineral salts to effect phase separation, the need to separate and recover both extraction solvents and solutes, and the need to remove large amounts of dissolved salts from aqueous and/or organic solvent fluids prior to reuse or disposal.

For example, in U.S. Pat. No. 4,508,929 ('929), D. C. Sayles, April 1985, “Recovery of Alcohol from a Fermentation Source by Separation Rather than Distillation”, Sayles describes the use of calcium chloride (CaCl₂) salt and diethyl ether solvent to extract and precipitate fermented ethanol (and presumably ethanol-soluble and diethyl ether-soluble fermented compounds) from a fermented liquid (i.e., beer). The '929 process uses a highly volatile and flammable diethyl ether to extract fermented ethanol from a fermentation liquid, followed by salting-out the ethanol from the diethyl ether by dissolving CaCl₂ into the diethyl ether-ethanol solution, which salts-out the ethanol from the diethyl ether layer. Following recovery of the ethanol from the diethyl ether, the ethanol solution is furthered processed by dissolving a second salt, sodium carbonate (Na₂CO₃), into solution to precipitate calcium carbonate (CaCO₄⬇) and sodium chloride (NaCl⬇) salts from the ethanol solution.

In summary, the main drawback of conventional SALLE processes thus described is that a lot of mineral salt is needed to enable the salting-out effect, resulting in a heavily saline wastewater which must be further treated prior to reuse or disposal. As with water-based extraction processes, concentrating and separating dissolved solutes such as mineral salts from water is both energy- and time-intensive, and results in significant operational cost impacts. As a result, conventional SALLE processes are considered a laboratory-scale extraction technique for preparing small-volume samples for analytic procedures.

Having described the relevant prior art, it is apparent that there is no one universal solvent extraction chemistry or technique, and each extraction technique discussed is constrained in one or more unique ways. However, each extraction technique discussed provides unique and desirable chemical, operational, and performance characteristics. Given this, an extraction technique is desirable which provides a combination of the desirable aspects and benefits, and with fewer drawbacks. For example, an extraction technique that provides environmental protection, human health and safety, and which can be optimized by conventional process intensification techniques is desirable. Further to this, a more capable extraction technique is desirable, which can more effectively extract polar and nonpolar compounds, ionic and non-ionic compounds, and low molecular weight and high molecular weight compounds to produce true full-spectrum extracts. Full-spectrum extracts are more efficiently and economically concentrated and separated using fractionation techniques such as thin-film vacuum distillation or chromatographic columns to produce discrete natural and pure compounds for use as pharmaceutical, nutraceutical, food, and beverage additives. Moreover, a smart and scalable extraction technique is desirable, which uses green extraction technology and is adaptable to a broader range of liquid and solid substances and extraction purposes.

Those skilled in the art of value extraction most commonly specialize in a particular extraction technology, for example, dense phase CO₂ (i.e., supercritical CO₂) extraction, organic solvent (i.e., ethanol) extraction, or aqueous (i.e., subcritical water) extraction. The prior art is replete with technical studies in those extraction techniques and little co-development or overlap exists between these technologies except in aspects such as solvent modification (i.e., solvent added to supercritical CO₂ or solvent added to a plant substance and then extracted with scCO₂) or employing one or more conventional extraction techniques in sequence.

Thus, there has been no significant development of an extraction technique that combines singular aspects of conventional extraction technology into a true hybrid process. This is evidenced by the dearth of prior art in either semi-aqueous or hybridized extraction techniques for recovering and processing a valuable extract from a plant material. According to Zhu, Z., et al. (University of Bath, U.K.), “A Review of Hybrid Manufacturing Processes—State of the Art and Future Perspectives”, International Journal of Computer Integrated Manufacturing, 26 (7), 2013, pp. 596-615 (Zhu et al.), although there is no specific consensus on the definition of the term ‘hybrid processes’, researchers have developed a number of approaches to combine different manufacturing processes with the similar objectives of improving surface integrity, increasing material removal rate, reducing tool wear, reducing production time and extending application areas. Zhu et al. further states that the initial purpose of developing hybrid manufacturing processes is to provide the advantages of constituent processes while minimizing their inherent drawbacks. There is a major need to establish the relationships between constituent processes and their respective control systems. This will largely determine the development of hybrid processes in the future. In this regard, the lack of hybrid extraction process development may be due to a lack of appreciation of the beneficial relationships between the various extraction techniques. Ultimately, a true hybrid extraction process must enable new opportunities and applications for extracting and processing an extract or analyte recovered from a substance which is not able to be performed economically (or not able to be performed at all) by conventional extraction processes on their own.

To address this opportunity, the present inventors have developed a novel extraction technique that adapts, modifies, and combines synergistic chemical, operational, and performance characteristics from several conventional extraction techniques into a true hybrid extraction process. The present invention hybridizes aspects of aqueous, organic solvent, dense phase CO₂, and SALLE extraction techniques into a tunable semi-aqueous extraction and extract recovery system. A central harmonizing component of the tunable extraction system is a unique CO₂ pressure-driven solvent expansion and salting-out phase separation process.

### SUMMARY OF THE INVENTION

The present invention provides liquid-liquid and solid-liquid extraction methods employing a semi-aqueous extractant, comprising water and one or more water-soluble or water-emulsifiable (WSWE) compounds, which is used simultaneously with a novel dense phase CO₂ salting-out assisted liquid-liquid extraction, extract concentration, and desolvation process. The present invention is useful for producing full-spectrum extracts derived from biomaterials such as plant materials for use as colorful, flavorful, or healthful additives in pharmaceuticals, nutraceuticals, beverages, or foods, and for producing extracts derived from environmental substances such as contaminated industrial wastewaters or polluted soils for quantitative analysis.

The present invention is very different from conventional liquid-liquid or solid-liquid extraction processes utilizing a dense phase CO₂ (i.e., liquid or supercritical carbon dioxide) or an organic solvent as a primary extractant, with or without co-solvents, used for example, to remove trace amounts of environmental organic pollutants from an aqueous solution (i.e., pesticide or gasoline residues), typically present at low parts-per-million (ppm) levels, or to extract valuable organic compounds from a plant material (i.e., vegetable oils, CBD, polyphenols), some of which are present at up to nearly 60% by weight.

A major distinction between the present invention and conventional dense phase CO₂ extraction processes is that an aqueous solution containing a water-soluble organic compound serving as a primary extractant (and a source of polar co-solvent for nonpolar CO₂) is selectively phase-separated or re-dissolved from or into water using CO₂ gas pressure. This process is visually evident within a Jerguson Gage operating at CO₂ gas pressures as low as 7 atm and up to vapor saturation pressure. Although gas phase CO₂ does not exhibit appreciable solvation power until a condensed phase (liquid) or high-density supercritical state is achieved, nonetheless high-pressure CO₂ gas significantly (and favorably) changes the physicochemical properties of solutes and solvents. In this regard, CO₂ gas driven liquid-liquid extraction and separation effects observed in dilute and concentrated aqueous solutions containing one or more WSWE compounds is believed to be caused by two phenomena. Firstly, pressure-driven CO₂ gas expansion of dissolved organic compounds contained in water dramatically changes their physicochemical properties such as polar cohesion energy. Secondly, pressure-driven CO₂ gas hydration (as well as CO₂ gas solvation) by water changes hydrogen-bonding cohesion energy of water. A lowering of WSWE (i.e., as solute) polar cohesion energy (δ_{P}) combined with a lowering of water (i.e., as solvent) hydrogen bonding cohesion energy (δ_{H}) by the dense phase CO₂ (i.e., as co-solvent) leads to phase separation or expansion/salting-out of WSWE to form a second or third solvent phase. CO₂ gas expanded and salted-out organic compounds may be decanted as a carbonated solvent-extract mixture. Alternatively, a CO₂ salted-out solvent-extract mixture may be selectively dissolved into a liquid phase CO₂ or supercritical state CO₂, if present as an upper solvent phase, and which is dependent on cohesion chemistry differences between the CO₂ salted-out organic compounds and dense phase CO₂.

Moreover, the unique phase separation and solvation phenomenon of the present invention are not observed in conventional organic solvent-aqueous extraction systems utilizing, for example, water-insoluble organic compounds such as vegetable oil, hexane, or methylene chloride. Still moreover, the CO₂-enabled salting-out method of the present invention is unique as compared to conventional salting-out liquid-liquid and solid-liquid techniques employing water, organic solvents, and mineral salts.

As such, a first and central aspect of the present invention is a CO₂-driven expansion and salting-out process called CO₂ salting-out assisted liquid-liquid extraction process or simply CO₂ SALLE process. In the CO₂ SALLE process, WSWE and additive compounds dissolved in water (all of which forming a primary extractant mixture) are selectively expanded and salted-out (phase separated or phase shifted) using dense phase CO₂ during or following a liquid-liquid or solid-liquid extraction process. The extraction solvent system comprising water-WSWE-additives-CO₂ is selectively adjusted (“tuned”) using dense phase CO₂ pressure and aqueous solution temperature, with the addition of process intensifiers such as ultrasonics, heat, and centrifugation, to provide an optimal extraction environment for either a liquid or solid substance. Dense phase CO₂ serves as an expansion and salting-out agent, co-extractant, and enables subsequent extract concentration and desolvation processes. Moreover, during biphasic and multiphasic semi-aqueous CO₂ SALLE processes of the present invention, first and second separated phases are produced, which comprise dense phase CO₂, WSWE, and extract. These first and second separated phases are called WSWE-rich and CO₂-rich CO₂ salted-out solvent mixtures, respectively. Dense phase CO₂ works synergistically with hot or cold water to plasticize and swell cuticular and cellulosic plant structures to improve CO₂ salted-out solvent mixture access to phytochemicals, and to enhance related solvent-extract diffusion phenomenon.

Still moreover, dense phase CO₂ is not a powerful or effective full-spectrum extraction solvent. As discussed under prior art, liquid and supercritical CO₂ solvent properties are remarkably like a fluorocarbon solvent chemistry. Dense phase CO₂ is a relatively nonpolar solvent with a low Kauri-Butanol (KB) value (<20) and extract solubility properties are based on fluid pressure and temperature. For example, dense phase CO₂ is a poor solvent for the many complex organic compounds (i.e., polycyclic and highly branched phytochemicals) encountered in botanical extraction applications, for example high molecular weight compounds with a large polar surface area (PSA) such as flavonoids. As a result, dense phase CO₂ (as a primary extractant) extractions are slower and more selective as compares to more powerful solvents such as ethanol. Moreover, a full-spectrum CO₂-based botanical extraction necessitates operating under supercritical conditions with higher temperatures and pressures, and the addition of a polar organic co-solvent or solvent modifier.

In this regard, organic compounds useful as co-solvent additives may be toxic, flammable, and are difficult to completely remove from extracted compounds. For example, commercially available ethanol is purposely denatured with up to 5% by volume of toxic organic compounds such as methanol, IPA, methyl ethyl ketone, and/or heptane to deter human consumption. These same denaturants ultimately contaminate botanical extracts. Moreover, co-solvent additives must be contained in a separate vessel and pumped into and mixed with a dense phase CO₂ prior to its introduction into the solid or liquid extraction system.

Given this, the CO₂ SALLE process preferably utilizes natural and purposefully formulated green and non-toxic semi-aqueous solutions, comprising water (typically most of a semi-aqueous composition) and one or a blend of WSWE and additive compounds, as a primary CO₂ salted-out extractant used in combination with dense phase CO₂ co-extractant; this process can also be used with synthetic and non-toxic solutions. Using novel CO₂-driven expansion and salting-out liquid-liquid extraction and adjunct methods and apparatuses of the present invention, the extraction process is significantly enhanced in terms of improved performance of the dense phase CO₂ liquid-liquid co-extraction chemistry and process, as well as improved quality of extracted compounds in terms of healthfulness, quantity, and value.

Exemplary CO₂ SALLE methods of the present invention utilize three basic components; 1) a solid and/or liquid substance to be extracted and co-extracted, 2) a semi-aqueous solution containing one or more water-soluble or water-emulsifiable (WSWE) compounds and additives, and 3) a dense phase CO₂ fluid. These components are collectively referred to herein as a “Tunable Extraction System”, detailed as follows:
1.1 A mass-regulated and particle size-regulated solid substance, for example botanicals, soils, microalgae, or animal tissue contained in a porous container (i.e., basket or cellulose extraction thimble) to be extracted (and co-extracted) by said semi-aqueous solution and dense phase CO₂; and/or
1.2 A volume-regulated liquid substance, for example alcoholic beverages, polluted wastewaters, or water-based extractants to be extracted (and co-extracted) by said semi-aqueous solution and dense phase CO₂; and either the solid substance or liquid substance may be co-located with said dense phase CO₂ or said aqueous solution;
2. A volume-regulated, concentration-regulated, and temperature-regulated semi-aqueous solution comprising water containing one or a blend of (preferably naturally derived) water-soluble or water-emulsifiable (WSWE) compounds and other additives, including hydrated and dissolved CO₂, used as a primary extractant to be selectively CO₂ salted-out, phase separated, and concentrated for extract recovery; and said liquid substance may be used as a semi-aqueous solution (i.e., high proof alcoholic beverage); and
3. A volume-regulated, pressure-regulated, and temperature-regulated dense phase CO₂ (CO₂ gas, solid CO₂, liquid CO₂, or supercritical CO₂) used primarily as a selective WSWE expansion and salting-out agent, and co-extractant; and used as an acidification and cooling agent for said semi-aqueous solution, liquid substance, and solid substance.

The CO₂ SALLE process is a central component of said tunable extraction system, which may be homogeneous or heterogeneous, and biphasic or multiphasic. The CO₂ SALLE process window of said tunable extraction system comprises a temperature range between −40° C. and 300° C. and a pressure range between 1 atm and 340 atm, and a preferred processing window comprising a temperature between −20° C. and 100° C. and a pressure range between 5 atm and 100 atm. Moreover, said semi-aqueous solution may contain between 0.1% and 95% by volume of one or a blend of (preferably naturally derived) WSWE and additive compounds.

A tunable extraction system utilizing the CO₂ SALLE process is useful as a stand-alone exhaustive extraction system or as an adjunct solvent-extract concentration and recovery process for water-based extraction processes and systems, for example, subcritical water extraction. A semi-aqueous solution containing water-soluble or water-emulsifiable (WSWE) compounds and optional additives (naturally present or purposefully added) is salted-out (phase-separated or phase-shifted) using hydrated and dissolved CO₂ gas species, inclusively referred to as aqueous CO₂ or CO_{2(aq)}, to produce predominantly aqueous and non-aqueous solvent phases, each of which contain compounds that are selectively soluble in said aqueous or non-aqueous solvent phases based on cohesion energy differences and partition coefficients. Further to this, the non-aqueous solvent phases may be selectively withdrawn and desolvated ex-situ using a simple CO₂-based capillary condensation technique or using a conventional dense phase CO₂ distillation and extract recovery technique. Alternatively, said aqueous and non-aqueous phases may be used in-situ and in combination as biphasic or multiphasic extraction mixtures to produce full-spectrum botanical extracts. Still moreover, the versatile CO₂ SALLE process of the present invention can be used to extract compounds (i.e., oils, metals, and other environmental pollutants) from solid phase or liquid phase environmental substances such as contaminated soils and industrial wastewaters for direct instrumental analysis.

In another aspect of the present invention, several exemplary stand-alone and hybrid (tunable) liquid-liquid and solid-liquid extraction systems, including a novel subcritical water-CO₂ SALLE extraction system, are described for removing both organic and inorganic compounds (collectively referred to as extracts herein) from liquid and/or solid substances. Exemplary tunable extraction systems include:
1. Stand-alone solid-CO₂ SALLE extraction, liquid-CO₂ SALLE extraction, and liquid-solid-CO₂ SALLE extraction, co-extraction, and extract infusion systems;
2. Hybridized dense phase CO₂—CO₂ SALLE systems for producing and delivering co-solvents and extract infusions (i.e., fermented ethanol and botanical extracts) to a solid-dense phase CO₂ extraction system and process (i.e., centrifugal liquid CO₂ extraction);
3. Hybridized subcritical water-CO₂ SALLE systems for concentrating and recovering extracts derived from a conventional solid-water extraction system and process (i.e., subcritical or pressurized water extraction); and
4. Hybridized CO₂ SALLE-analysis systems for providing solid or liquid substance extraction and instrumental chemical analysis (i.e., light-induced fluorescence spectroscopy).

Moreover, another aspect of the present invention is “Smart Extraction”. Smart extraction as illustrated herein employs an analytical instrumental method to monitor dissolved extract concentrations contained in an aqueous extractant phase, semi-aqueous extractant phase, or CO₂ extractant phase to optimize and control the exemplary extraction, extract concentration, and extract recovery processes described herein. For example, many organic compounds present in biomaterials are unsaturated organic compounds that will fluoresce when excited by ultraviolet (UV) light. Unsaturated extractable compounds contain one or more unsaturated carbon-carbon bonds (i.e., double or triple bonds). As such, specific unsaturated extractable compounds that do fluoresce are used as “chemical markers” to optimize biomaterial extraction performance and to monitor the progress of exemplary biomaterial extraction and biomaterial extract recovery processes of the present invention. For example, a laser- or light-induced fluorescence (LIF) smart extraction technique is described herein for detecting and quantifying changes in an exemplary chemical marker concentration, d-limonene; a natural unsaturated terpenoid found in many biomaterials. Using LIF, the d-limonene concentration is measured in-situ and in real-time within a particular extraction fluid phase to determine an increasing or decreasing concentration level.

Another aspect of the present invention is “Process Adaptability”. Process adaptability as illustrated herein is the ability to integrate and hybridize the present invention with conventional extraction processes such as CO₂ phase shifting extraction ('366), centrifugal CO₂ extraction ('112), and dense fluid treatment and recycling ('362) technologies developed by the first-named inventor of the present invention, and discussed under the prior art. Moreover, process adaptability describes the ability to integrate and hybridize the present invention with conventional water-based extraction processes such as subcritical water extraction (SWE), for example subcritical water extraction of phytochemicals ('190) and pressurized low polarity water extraction apparatus ('948), discussed under the prior art. With regards to conventional SWE, the present invention describes unique and beneficial CO₂-solvent physicochemical adaptations and modifications, termed “modified SWE” or “MSWE” herein. Modified SWE solvents and processes described herein utilize water-soluble or water-emulsifiable (WSWE) compounds which lower the cohesion energy, and processing temperature and pressure. Moreover, modified SWE processes utilize alcoholic beverages as subcritical water extraction solvents. Finally, modified SWE processes utilize dense phase CO₂ as a vapor pressure control agent and solvent modifier.

Another aspect of the present invention is “Scalability”. The present invention can be implemented as a small-scale tabletop botanical extraction system for research and development or single consumer use. For example, a benchtop system uses disposable 100% pure cellulose thimbles containing the botanical material to be processed, like a Soxhlet extraction system. Moreover, the present invention can be implemented as an on-line environmental sampling and analysis system, for example an in-situ method for sampling, extracting, and quantifying organic pollutants contained in an industrial wastewater effluent. Alternatively, the present invention can be scaled to process much larger volumes of dry ground botanical material, for example capacities of 25 cubic feet or more, using for example a centrifugal extraction system ('112) developed by the first-named inventor, and discussed under prior art.

Finally, another aspect of the present invention is “Minimization”. The present invention minimizes the use of toxic or hazardous organic solvents, and energy and time intensive monophasic solvent extraction systems and processes, using novel and green hybrid Water-CO₂-natural/human safe organic solvent extraction chemistries in combination with novel CO₂ SALLE co-extraction, extract concentration, and extract recovery processes. Moreover, the present invention supports and enables new emergent green water-based extraction processes such as subcritical water extraction.

### BRIEF DESCRIPTION OF THE DRAWINGS

The accompanying drawings, which are incorporated in and constitute a part of the specification, illustrates the present invention and, together with the description, serve to exemplify the principles, practices, benefits, and novelty of the present invention.

FIG. 1A and FIG. 1B provide digital photos taken during experimental testing using a dilute aqueous alcohol (DAA) solution, a 90%:10% v:v Water:IPA solvent system, illustrating selective CO₂ salting-out solvent effects used to enhance liquid-liquid and solid-liquid extraction processes of the present invention. FIG. 1C and FIG. 1D are graphs describing the solubility of CO_{2 (g)} in water with respect to water temperature and CO₂ pressure, respectively.

FIG. 2 provides digital photos taken during experimental testing using a concentrated aqueous alcohol (CAA) solution, a 90%:10% v:v IPA:Water solvent system, illustrating CO₂ solvent expansion and Marangoni-Rayleigh convection effects used to enhance liquid-liquid and solid-liquid extraction processes of the present invention.

FIG. 3A is a schematic describing key aspects of an exemplary CO₂ SALLE apparatus and process of the present invention. FIG. 3B is a diagram related to FIG. 3A describing tunable solvent phase and solubility properties of the present invention as used in a liquid-liquid or solid-liquid extraction process. FIG. 3C is a diagram related to FIGS. 3A and 3B describing solubility properties of exemplary plant structures. FIG. 3D is a diagram related to FIGS. 3A and 3B describing solubility properties of exemplary phytochemicals contained in exemplary plant structures of FIG. 3C.

FIG. 4 is a flowchart describing exemplary aspects of a tunable extraction system used in combination with a CO₂ SALLE process of FIGS. 3A and 3B.

FIG. 5A provides diagrams describing four exemplary CO₂ SALLE methods derived from FIGS. 3A and 3B, and FIG. 4 used in liquid-liquid and solid-liquid extraction schemes. FIG. 5B provides a diagram describing an exemplary CO₂ SALLE method and process for performing a cluster extraction.

FIG. 6A is a chart contrasting and comparing the change in total cohesion properties versus temperature for unmodified (water only) and modified subcritical water extraction (MSWE) solutions containing ethanol. FIG. 6B is a chart contrasting and comparing the change in total cohesion energy versus semi-aqueous solution temperature for a modified subcritical water extraction (MSWE) of FIG. 6A with integration of an exemplary CO₂ SALLE process of FIG. 3B.

FIG. 7 is a schematic describing the use of the exemplary CO₂ SALLE processes of FIGS. 3A and 3B in an exemplary semi-aqueous solid-liquid subcritical water (MSWE) extraction system, including a means for recycling process fluids, and a means for monitoring the progress of the CO₂ SALLE process using exemplary analytical chemical processes.

FIG. 8A is a schematic showing the integration of an exemplary light-induced fluorescence (LIF) smart extraction monitoring and control system of the present invention. FIG. 8B is an exemplary LIF spectrogram for the smart extraction monitoring and control system described under FIG. 8A. FIG. 8C is an exemplary solvent extraction curve showing the general profile for an optimized smart extraction process using an exemplary d-limonene marker chemical.

FIG. 9 is a schematic describing an exemplary CO₂ solid-gas aerosol assembly for use a cooling device and as a desolvation device.

FIG. 10A is a schematic describing a novel use of an ozonation process to alter the chemistry of beverage and biomaterial extracts to produce oxygenated tinctures or concentrates for producing bio-based extract-infused emulsions. FIG. 10B describes the effect of ozonation of an exemplary plant extract, oleic acid, including changes in chemical and physical properties which enable improved emulsification.

FIG. 11 provides a schematic and flowchart describing an exemplary hybrid cannabis decarboxylation and extraction process utilizing a semi-aqueous extractant, under subcritical water temperature and pressure conditions, followed by a CO₂ SALLE process.

### DETAILED DESCRIPTION OF THE INVENTION

In the description that follows, like parts are indicated throughout the specification and drawings with the same reference numerals, respectively. The figures are not drawn to scale and the proportions of certain parts have been exaggerated for convenience of illustration.

The liquid-liquid phase separation phenomenon, which forms the basis of exemplary CO₂ SALLE methods and apparatuses detailed herein, was unexpectedly observed by the first-named inventor during dense phase CO₂-liquid solubility experiments employing a high pressure Jerguson Gage. In one experiment among many involving natural oils and alcohols, the Jerguson Gage was partially filled with an aqueous solution comprising 10% isopropanol (IPA) and 90% deionized water (H₂O), considered a dilute aqueous alcoholic (DAA) solution. The purpose of this extraction test was to determine the volume of IPA that could be extracted from a substantially water-based solution (aqueous phase). IPA is a water-soluble or water emulsifiable (WSWE) organic compound useful for practicing the present invention. While applying an incremental and increasing CO₂ gas pressure gradient over said aqueous solution ranging from 1 atm (ambient pressure, no CO₂ gas present) to 61 atm (CO₂ gas saturation conditions) at a temperature of approximately 20° C., an IPA phase was formed (visually evident) at 7 atm and gradually increased in volume above the aqueous phase as CO₂ pressure increased. This phase separation was also marked by a gradual decrease in the level of the aqueous phase meniscus (or interphase). Additionally, the volume of the IPA phase decreased, and the volume of the aqueous phase increased as the CO₂ gas pressure was decreased, but more slowly presumably due to IPA-water density differences and CO₂ gas evolution (effervescence), demonstrating the capability to control the IPA-water phase separation process reversibly using CO₂ gas pressure.

Further development determined that injecting solid-gas CO₂ aerosol through the lower port of the Jerguson Gage using a small capillary tube significantly improved the liquid-liquid phase separation process through improved mixing action and lower solution temperature. Lower solution temperature increased CO₂ solubility levels and the resulting CO₂ froth rising through the aqueous solution quickly transferred and segregated the IPA solvent phase to form an upper surface layer. As such, this technique is a preferred CO₂ injection method in the present invention. Upon reaching CO₂ gas saturation conditions (>54 atm at 20° C.), a water-insoluble liquid CO₂ phase was formed above the aqueous solution and the IPA phase. Following this, a portion of the IPA solvent phase diffused and dissolved into the liquid CO₂ phase, indicating that the small amount of liquid CO₂ phase quickly reached a saturation condition with the IPA phase. As more liquid CO₂ was added to the Jerguson Gage, more IPA dissolved into the liquid CO₂ phase.

Subsequently, IPA dissolved in the liquid CO₂ was recovered by withdrawing the upper liquid CO₂ phase and condensing same into a solid phase CO₂-IPA mixture using a 6-foot section of 0.020-inch (inside diameter) polyetheretherketone (PEEK) capillary tubing. The PEEK capillary condenser technique is a simple CO₂ condensation process developed by the first-named inventor in the early 1990's, described in prior art U.S. Pat. No. '154 et al., and is uniquely adapted to the present invention as a novel near-cryogenic phase separation and extract recovery technique.

Moreover, testing with dilute and concentrated acetone-water solutions produced similar results as IPA-water solutions. Still moreover, additional testing confirmed that the CO₂ SALLE process was very effective in separating and recovering fermented and distilled ethanol (and Raspberry flavonoids) from a commercially available Raspberry-flavored 70 Proof Vodka (35% fermented EtOH by volume), as well as ethanol (and Whiskey flavonoids) from a commercially available 80 Proof Bourbon Whiskey (40% fermented EtOH by volume). Flavonoids represent a complex mixture of polyphenolic compounds which are not appreciably soluble in nonpolar solvents such as liquid and supercritical carbon dioxide.

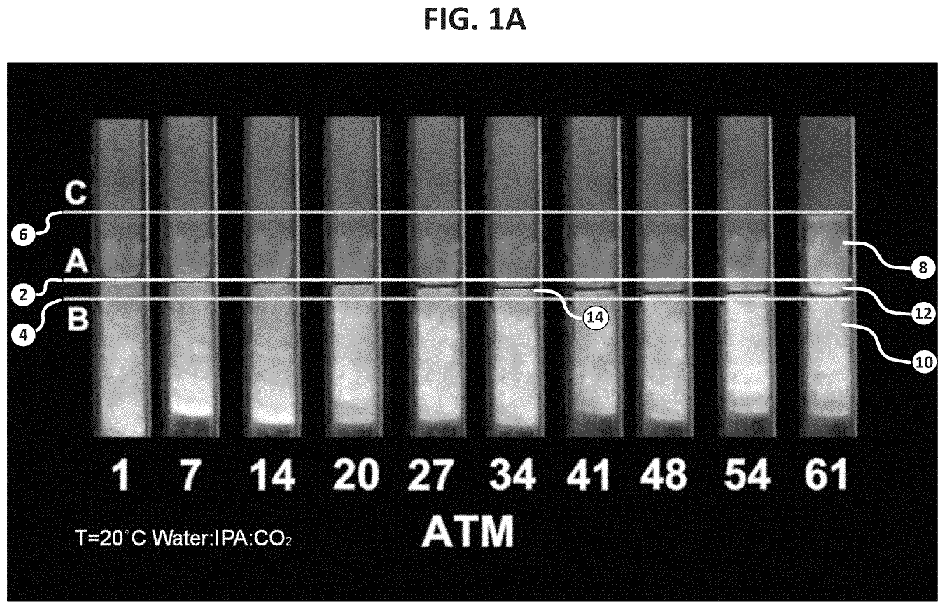

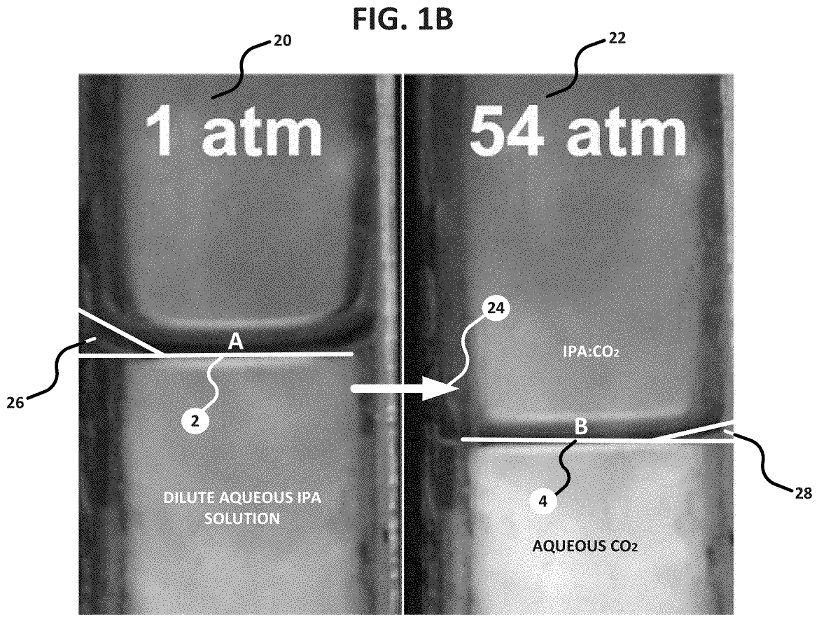

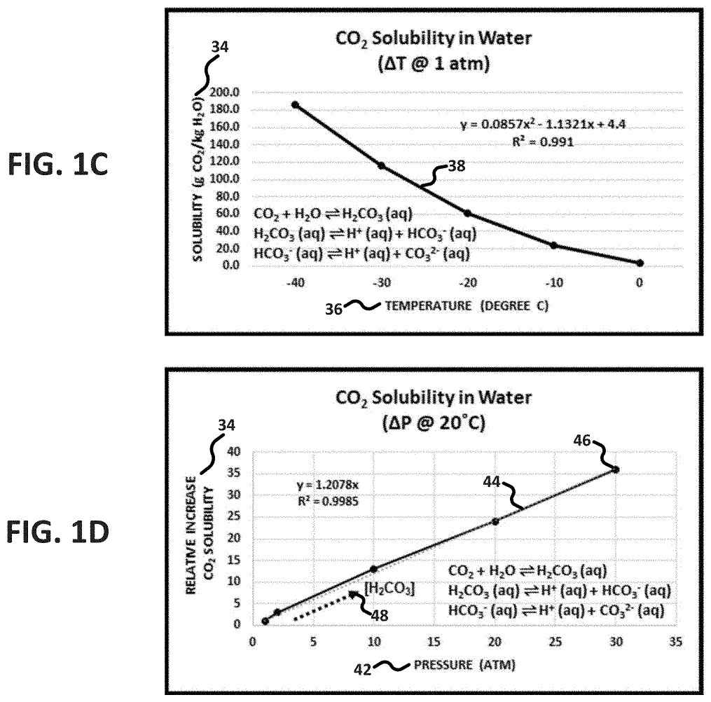

FIG. 1A and FIG. 1B provide digital photos taken during experimental testing using a dilute aqueous alcohol (DAA) solution, a 90%:10% v:v Water:IPA solvent system, illustrating selective CO₂ salting-out solvent effects used to enhance liquid-liquid and solid-liquid extraction processes of the present invention.

Experiments were performed by the present inventors using a high pressure Jerguson Gage (Series 40, Transparent Rectangular Sight Glass, 5000 psi @ 100° F. rating, Clark-Reliance, Strongsville, Ohio). The Jerguson Gage contains threaded top and bottom ports for implementing piping, pressure Gage, and inlet-outlet valves for facilitating filling, pressurization, and draining test solvents and CO₂ gas. The Jerguson Gage was filled with a fixed volume (about 50% of Gage capacity) of aqueous solvent solution comprising 90%:10% (by volume) Water:IPA, also described as a dilute aqueous alcohol (DAA) solution herein, following which pressure-regulated CO₂ gas derived from a steel cylinder of high pressure liquid CO₂ was introduced into the top of the Jerguson Gage containing said fixed volume of aqueous organic solvent in discrete and increasing pressurization increments or stages from 1 atm to 61 atm. Prior to and following each pressurization stage, a fixed-position digital camera was used to take a photograph of the same liquid-vapor level region within the Jerguson Gage, supplemented by low-level backlight illumination using a microscope light source positioned behind the transparent high-pressure window of the Jerguson Gage.

Now referring to FIG. 1A, ten (10) digital photographs were taken during each pressurization stage from 1 atm (S.T.P., no CO₂ gas present) to 61 atm (saturated CO₂ liquid-vapor formation at ambient temperature). As can be seen in FIG. 1A, from a CO₂ pressure of 1 atm to 54 atm, the Water:IPA liquid-vapor level decreases steadily and consistently with each pressure step starting from Level A (2) at 1 atm to Level B (4) at 61 atm. As shown in FIG. 1A, the water phase salts-out and separates the more dilute IPA phase selectively and uniformly through the entire CO₂ (gas→vapor) pressurization range, evidenced by the decreasing solution level. The density (relative concentration) of CO₂ for each pressure step is given in Table 3. In this regard, the level of the DAA solution steadily decreases with increasing CO₂ pressure (concentration), producing a biphasic separation between the lower phase (predominantly aqueous) and the emerging upper phase (predominantly non-aqueous). It can also be seen in FIG. 1A, the emergent CO₂₍ᵥ₎-IPA phase exhibits Marangoni-Rayleigh convective effects, with the expanded/salted-out IPA exhibiting capillary rise along the interior of the sight glass. This mass transfer is caused by large surface tension and density gradients between the liquid IPA and dense CO₂ vapor.

TABLE 3
CO₂ Pressure vs. Density (Concentration) @ T = 20° C.

| Pressure | |
|---|---|
| P_{CO2} - | Density |
| atm | σ - g/cm³ |
| 1 | 0.002 |
| 7 | 0.013 |
| 14 | 0.027 |
| 20 | 0.041 |
| 27 | 0.058 |
| 34 | 0.078 |
| 41 | 0.101 |
| 48 | 0.131 |
| 54 | 0.166 |
| 61 | 0.787 |

The IPA phase (an exemplary WSWE compound) is expanded and salted-out to the surface of the Water:IPA solution due to both density and solubility parameter (cohesion energy) differences. The IPA phase increases in volume during CO₂ expansion and as more CO₂-based species are formed within the semi-aqueous solution. Finally, the emergent IPA phase is (selectively) dissolved into a liquid carbon dioxide phase formed at a CO₂ pressure above 54 atm, evidenced by the appearance of the liquid CO₂ interphase at level C (6). Again, referring to FIG. 1A, it can be seen that the CO₂-IPA solution (8) formed is turbulent and not homogeneous, indicative that an IPA-saturated liquid CO₂ solution (8) has formed. This is due to an excess volume of expanded and salted-out IPA phase. Saturated liquid CO₂ and IPA have similar densities and are partially miscible. CO₂ gas behaves as a hydrated or dissolved solute in water and as an expansion agent or solvent (liquid/SCF) for IPA, and produces a stratified multiphasic solution as follows:

a) Aqueous CO₂ (CO_{2(aq)} Phase (10)

b) CO₂ Expanded IPA Phase (12); and

c) Saturated Liquid CO₂-IPA Phase (8)

The aqueous CO₂ phase (δ_{T}—47.8 MPa^{1/2}) forms a lower phase with a interphase at level B (4), salting-out the CO₂ Expanded IPA Phase (δ_{T}˜23.6 MPa^{1/2}) to the surface due to an approximate 20% difference in density (water—1.0 g/cm³ and IPA—0.78 g/cm³ with a interphase at level A (2), and forms a saturated liquid CO₂-IPA upper phase (δ_{T}˜20 MPa^{1/2}) at 61 atm with a interphase at level C (6).

Finally, and again referring to FIG. 1A, the semi-aqueous solution level progressively decreases from a starting Level A (2) to a stopping Level B (4), evidenced by the lowering of the IPA-H₂O interphase, and indicated by a thin dashed line (14) representing an approximate middle point for the progressive change in the semi-aqueous solution meniscus at 34 atm CO_{2(g)}. Given this, during operation of the CO₂ SALLE process, Level A (2) is used as a pressure vessel fill and withdrawal marker for the semi-aqueous solution and CO₂ salted-out CO₂-WSWE solution, respectively, for example using an optical level sensor such as the high pressure ELS Series electro-optical level sensors available from Gems Sensors and Controls, Plainville, Conn. Following a CO₂ SALLE process, the CO₂-WSWE solution containing extracts, both solvated and desolvated extracts, produced between Level B (4) and Level A (2) (called a “WSWE-rich CO₂ salted-out solvent mixture” herein) may be withdrawn for desolvation and extract recovery operations. Alternatively, the CO₂-WSWE solution containing extracts produced between Level A (2) and Level C (6) (called a “CO₂-rich CO₂ salted-out solvent mixture” herein) may be withdrawn for desolvation and extract recovery operations.

Now referring to FIG. 1B. FIG. 1B is a side-by-side comparison of the same interphase region from FIG. 1A showing changes to the composition and liquid-vapor level between 1 atm (20) and 54 atm (22) of CO₂ pressure. As shown in FIG. 1B, the starting liquid-vapor level A (2) at S.T.P., 1 atm and 20° C. (0.002 g CO₂/cm³), decreases as the IPA (WSWE compound) (24) is salted-out from the dilute aqueous alcohol solution to produce a lower liquid-vapor Level B (4) at 54 atm CO₂ pressure (0.166 g CO₂/cm³). Moreover, as shown in FIG. 1B, a significant decrease in surface tension occurs, evidenced by comparing the interphase contact angle (26) at Level A (2) with the interphase contact angle (28) at Level B (4).

Finally, interfacial turbulence caused by Marangoni-Rayleigh instability during the physical absorption and desorption of carbon dioxide into and from non-aqueous solvents (i.e., WSWE compounds) salted-out from a semi-aqueous solution. Marangoni instabilities depend on the change of interfacial tension and Rayleigh instabilities on the change of liquid densities with solute concentration. Such flows develop increasingly complex cellular or wavy patterns. The presence of interfacial turbulence significantly enhances mass transfer rates in liquid-liquid and solid-liquid extraction processes.

The observed Marangoni-Rayleigh convections (turbulences) observed in the CO₂ SALLE process are due to differences in interfacial surface tensions and densities, but visible and unique (microscopic or macroscopic) pattern formations within the interphases, as evidenced by light transmission changes from transparent to translucent, are presumably due to cohesion energy differences between the CO₂, WSWE solutes, water, and gravity. As such, combined with the observations of Sun et al., it can be conjectured that the visible interfacial turbulences described herein under FIGS. 1A and 1B are a result of (1) WSWE solute(s) salting-out from the aqueous phase and (2) CO₂ absorption into (and expansion of) the emergent WSWE phase, and all of which is throttled by the CO₂ gas absorption into the liquid water phase. Moreover, the volumes and levels of the interphases formed are selectively and adjustably controlled using CO₂ pressure (i.e., concentration), WSWE cohesion energy and concentration, and aqueous solution temperature.

Having discussed CO₂ salting-out behavior of the exemplary CO₂ SALLE process, following is a more detailed discussion of CO₂ solubility and acidification aspects under FIGS. 1C and 1D.

The IPA salting-out effects described under FIGS. 1A and 1B are directly proportional to the CO₂ concentration within the semi-aqueous solution. In this regard, CO₂ pressure and semi-aqueous solution temperature drive aqueous CO₂ concentration levels. FIG. 1C and FIG. 1D are graphs describing the solubility behavior of CO₂ in water with respect to water temperature and CO₂ pressure, respectively. FIGS. 1C and 1D have been adapted from CO₂-water solubility data available from Engineering ToolBox, (2006). Carbon Dioxide Properties. [online] Available at: https://www.engineeringtoolbox.com/carbon-dioxide-d_1000.html [Accessed 20 02 2020], and Engineering ToolBox, (2008). Solubility of Gases in Water. [online] Available at: https://www.engineeringtoolbox.com/gases-solubility-water-d_1148.html [Accessed 20 02 2020].

Water content levels in semi-aqueous solutions containing organic solvents such as alcohols (i.e., ethanol, methanol, IPA) can significantly impact botanical extraction performance, and particularly at lower solution temperatures and for recovery of relatively nonpolar botanical compounds such as terpenes and cannabinoids. Degradation of extraction performance is attributed to unfavorable changes in chemical and physical factors such as increased cohesion energy (i.e., lower solubility of organic compounds) and increased surface tension (i.e., poor wetting of botanical surfaces). Semi-aqueous compositions of the present invention can range between 0.1% and 95% WSWE content, and preferably between 0.1% and 30% WSWE compounds by volume. As such, the majority component of exemplary semi-aqueous compositions of the present invention is water. In this regard, the present invention uniquely enables the use of water-concentrated semi-aqueous solutions as effective biphasic and multiphasic extractants for botanical compounds possessing extremely limited water solubility (i.e., nonpolar terpenoids) and organic compounds exhibiting higher water solubility (i.e., polar flavonoids).

Moreover, exemplary CO₂ SALLE processes of the present invention can be operated at lower temperatures and higher pressures, enabled by a near-cryogenic CO₂ gas-solid aerosol to produce CO₂ saturation with lower solution temperature in combination with (preferably) elevated CO₂ pressures using autogenous or mechanical pressurization. In this regard, FIG. 1C is a graph showing the solubility of CO₂ (34) versus water temperature (36) at 1 atm. As shown in FIG. 1C, CO₂ solubility increases with decreasing water temperature (38), saturating water (H₂O) with hydrated and dissolved CO₂ species (CO_{2(aq)}: bicarbonate ions (HCO₃⁻_{(aq)}), carbonate ions (CO₃^{2-(aq)} and carbonic acid (H₂CO_{3(aq)}). CO₂ solubility increases at a rate of approximately 5 g CO₂/kg H₂O/° C. between 0° C. and −40° C. at 1 atm, or 5×. More significantly, and now referring to FIG. 1D, CO₂ solubility in water (40) increases with applied CO₂ pressure (42) linearly (44) between 1 atm and 30 atm at 20° C. CO₂ solubility increases 36× at 30 atm (46). More significantly, at CO₂ pressures above 2 atm, appreciable amounts of carbonic acid begin to form (48), and it is thought that carbonic acid (CO₂+H₂O←→H₂CO₃) and hydrated bicarbonate ion (H₂CO₃←→H⁺+HCO₃⁻) formation are the principle aqueous species. As can be seen, lower operating temperatures with moderate CO₂ pressures are preferred in the present invention as these conditions favor the CO₂ SALLE process, and particularly the expansion and salting-out aspects to produce two or more solvent phases.

Also, liquid phase water is only sparingly soluble (as a solute) in liquid CO₂. However, CO₂ (as a hydrated and ionized solute, and dissolved gas) is variably soluble within a semi-aqueous solution based on both CO₂ pressure (P) and temperature (T), as well as WSWE compound composition. At P-T operating ranges employed in the present invention, significant differences exist between the aqueous phase and dense phase CO₂ in terms of density (σ) and total Hansen Solubility Parameter (δ_{T}). For example, at 80 atm and 0° C., liquid CO₂ has a σ=0.96 g/ml and a δ_{T}=17.9 MPa^{1/2} and liquid water has a σ=1 g/ml and a δ_{T}=47.9 MPa^{1/2}. As CO₂ gas is compressed into an aqueous solution at a pressure greater than approximately 54 atm at room temperature a water-insoluble liquid CO₂ phase forms above the aqueous solution.

Simultaneously with this, and in accordance with Equation 1 (Eq. 1), a P-T controlled portion of the CO₂ dissolves (as a gas) into said aqueous solution to form hydrated and ionized CO₂ species: P-T adjustable amounts of dissolved carbonic acid (H₂CO₃), bicarbonate anion (HCO₃⁻), and carbonate anion (CO₃²⁻), collectively referred to herein as aqueous CO₂ or CO_{2 (aq)}.

CO₂+H₂O↔H₂CO₃↔HCO₃⁻+H⁺↔CO₃²⁻+2H⁺ (Eq. 1)

In the present invention, CO_{2 (aq)} is uniquely employed to complex water molecules to assist CO₂ expansion with selectively salting-out WSWE and solvent-soluble compounds (i.e., extracts) dissolved in a semi-aqueous solution. As described herein with respect to Eq. 1, this is a result of the hydration of CO₂ gas molecules and the formation of carbonic acid, believed to be one of the major drivers of the CO₂ SALLE process at elevated pressures, and subsequent ionization of carbonic acid to form bicarbonate and carbonate anions. CO_{2 (aq)} species are very stable, even at high temperature. However, the concentration and stability of hydrated CO_{2 (aq)} complexes are CO₂ pressure and solution temperature dependent.

In Garand, E. et al., “Infrared Spectroscopy of Hydrated Bicarbonate Anion Clusters: HCO₃—(H₂O)₁₋₁₀”, J. AM. CHEM. SOC. 2010, 132, 849-856 (Garand et al.), spectroscopic evidence was presented that showed water molecules strongly associate and complex with the negatively charged CO₂ moiety of the HCO₃⁻ anion. The most stable isomer comprises n=4 water molecules, a four-membered ring with each water molecule forming a single H-bond with the CO₂ moiety. A second hydration shell forms at n=6 water molecules and forms a total hydration shell comprising ten (10) water molecules. Further to this, in Zilberg, S., et al., “Carbonate and Carbonate Anion Radicals in Aqueous Solutions Exist as CO₃(H₂O)₆²⁻ and CO₃(H₂O)₆⁻ Respectively: The Crucial Role of the Inner Hydration Sphere of Anions in Explaining Their Properties”, Phys. Chem. Phys. Chem., 2018, 20, 9429-9435 (Zilberg et al.), spectroscopic evidence was presented demonstrating that the carbonate anion radicals form strong six (6) member hydration shells. Finally, in Wu, G. et al., “Temperature Dependence of Carbonate Radical in NaHCO₃ and Na₂CO₃ Solutions: Is the Radical a Single Anion?”, J. Phys. Chem. A, 2002, 106, 2430-2437 (Wu et al.), Wu et al. determined that carbonate and bicarbonate anions dissolved in supercritical water are very stable. Wu et al. used pulsed radiolysis to produce and measure carbonate radical concentrations formed from these supercritical water-salt solutions and showed no appreciable change in the carbonate-bicarbonate anion system at temperatures as high as 400° C.

Moreover, with an increasing concentration of CO_{2 (aq)}, the pH of an (unbuffered) aqueous solution decreases. As such, small amounts of associated water co-extracted with a WSWE compound and solubilized into either an aqueous or dense phase CO₂ extraction solvent phase will be weakly acidic due to the presence of excess carbonic acid at high CO₂ gas saturation. In Peng, C. et al., “The pH of CO₂-saturated Water at Temperatures between 308 K and 423 K at Pressures up to 15 MPa”, J. of Supercritical Fluids 82 (2013) 129-137 (Peng et al.), it was determined that pH was dependent upon temperature, pressure, and CO₂ gas solubility in water (H₂O) at temperatures between 308 K (35° C.) and 423 K (150° C.) and pressure up to 15 MPa (148 atm, 2175 psi). For the pH measurements, liquid CO₂ was selectively pressurized into a temperature-controlled water sample using a precision syringe pump (Teledyne Isco, Model 100DM). The CO₂+H₂O system was contained in a pressure vessel outfitted with pressure, temperature, and pH sensors. The results of this study showed that pH decreases along an isotherm in proportion to −log 10(x), where x is the mole fraction of dissolved CO₂ in H₂O. The pH for the CO₂+H₂O system at 35° C. ranged from about pH=3.8 to pH=3 between 60 psi and 2000 psi. As expected, increasing temperature reduced CO₂ gas solubility, which increased pH values. The pH for the CO₂+H₂O system at 150° C. ranged from about pH=4.0 to pH=3.5 between 145 psi and 2000 psi.

Processing (salting-out) temperatures for exemplary CO₂ SALLE methods of the present invention are preferably less than 30° C. to produce a liquid CO₂ phase above the semi-aqueous solution. As such, the pH range at these operating temperatures (at elevated pressures) is estimated to be between pH=3.5 and pH=2 due to the much higher CO₂ gas solubility levels. In the present invention, this aspect is beneficial for improving the extraction performance of natural products containing target compounds with functional groups behaving as acids or bases, for example CBDA and THCA extracts found in cannabis. For example, in Heydari, R. et al., “Simultaneous Determination of Saccharine, Caffeine, Salicylic acid and Benzoic acid in Different Matrixes by Salt and Air-assisted Homogeneous Liquid-Liquid Extraction and High-Performance Liquid Chromatography”, J. Chil. Chem. Soc., 61, No. 3, 2016 (Heydari et al.), it was determined that sample pH has a significant influence on the extraction efficiency of organic extracts with acidic or basic functional groups and that the optimal extraction efficiency occurs at a pH=3.

In the present invention, CO_{2 (aq)} demonstrates strong and selective salting-out behavior in aqueous solutions containing WSWE compounds. For example, dissolved organic compounds (i.e., fermented ethanol (EtOH) and EtOH-soluble compounds) are adjustably “salted-out” from aqueous solutions using pressure- and temperature-controlled concentrations of CO_{2 (aq)}. The amount of salted-out organic solvent is directly proportional to the concentration of CO_{2 (aq)}. Moreover, injecting CO₂ into the bottom of an aqueous phase containing a WSWE compound produces turbulence and cooling actions through CO₂ solid phase sublimation and Joule-Thomson expansion effects, which enhances CO₂ gas saturation and mixing during salting-out of the organic solvent(s). Turbulence enhances transfer of organic compounds into the salted-out organic solvent phase and assists the rise and separation of the salted-out solvent phase (and solvent-soluble compounds) to the surface of the aqueous solution as the CO₂ rises, a process called dissolved gas flotation.

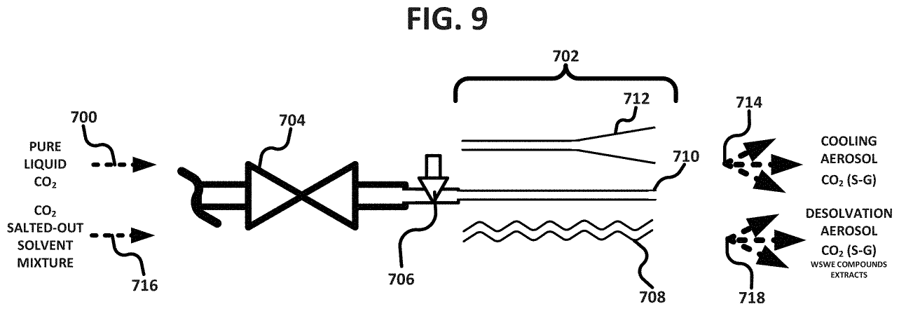

The CO₂ SALLE process can be operated at elevated temperatures and pressures, for example above the critical point for pure CO₂ (Tc=31° C., Pc=73 atm). This aspect is useful for performing hybrid subcritical water-CO₂ SALLE extraction processes described herein utilizing pressurized and heated water-based extraction solvents, for example using hydroethanolic mixtures to extract a solid substance at 80° C. in a subcritical water extraction process. Higher aqueous solution temperatures require higher dense phase CO₂ pressures to produce efficient and effective expansion and salting-out effects. Moreover, using semi-aqueous solutions containing WSWE compounds such as surfactants at temperatures above their surfactant cloud point temperature (T_{c}) can cause the surfactants to prematurely separate from the aqueous solution prior to the CO₂ SALLE process. This can affect the performance of the extraction process and complicate follow-on desolvation and extract recovery processes. As such, semi-aqueous extraction solutions are preferably cooled to below 50° C. prior to adding WSWE compounds such as these to maximize CO₂ expansion, ionization, and hydration effects, and to minimize complications during the CO₂ SALLE process. For example, a subcritical water extractant operating at 100° C. may be first cooled using a conventional heat exchanger and then further cooled and saturated with CO₂ using the novel near-cryogenic CO₂ solid-gas aerosol injection process described under FIGS. 9A and 9B. Following cool down and CO₂ saturation, a pre-determined and formulated WSWE mixture is injected and mixed into the subcritical water extractant and autogenously or mechanically pressurized to salting-out conditions using the CO₂ SALLE process.

Subsequently, CO₂ salted-out WSWE compounds containing solubilized extracts (also collectively referred to as CO₂ salted-out compounds) may be withdrawn as a CO₂ gas pressurized and carbonated solvent phase from the top-layer of the aqueous solution. Alternatively, CO₂ salted-out compounds (i.e., solvents, surfactants, and extracts) may be solubilized (partially or completely) within a top-layer liquid (or supercritical) CO₂ phase and used as solvent blend for an extraction process or desolvated to recover the CO₂ and CO₂-salted-out compounds. In an exemplary separation process of the present invention, CO₂ salted-out organics and liquid CO₂ are first separated from the top-layer of the aqueous phase and then phase-separated or desolvated using a near-cryogenic (−78° C.) crystallization process. Other novel CO₂ SALLE methods discussed herein include an in-situ aqueous botanical solid extraction and extracted oil flotation process. Finally, the CO₂ salted-out organic compounds (extracts) may be analyzed using an in-situ analytical chemical process such as light-induced fluorescence or injected directly into an external analytical chemical process instrument such as a high-performance liquid chromatography system or liquid density measurement system.

Having discussed exemplary aspects of phase separation phenomenon related to aqueous CO₂ solubility behavior under FIGS. 1A, 1B, 1C, and 1D, following is a discussion of Marangoni-Rayleigh convection phenomenon associated with the CO₂ SALLE process.

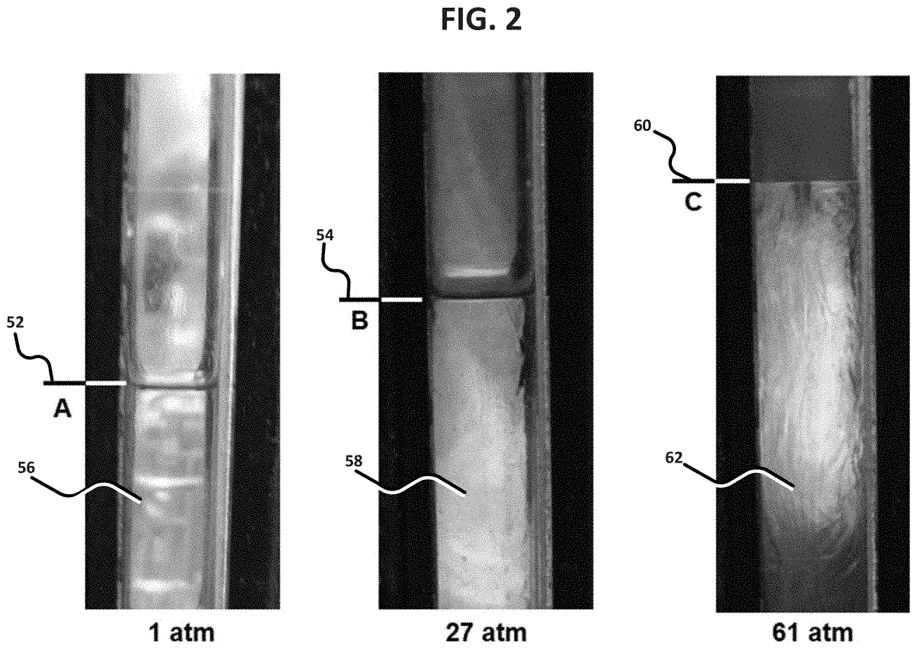

FIG. 2 provides digital photos taken during experimental testing using a concentrated aqueous alcohol (CAA) solution, a 90%:10% v:v IPA:Water solvent system, illustrating CO₂ solvent expansion and Marangoni-Rayleigh convection effects which enhance liquid-liquid and solid-liquid extraction processes of the present invention.

A central aspect of the CO₂ SALLE process is the use of CO₂ pressure and semi-aqueous solution temperature to selectively salt-out one or more WSWE compounds dissolved in a semi-aqueous solution to provide a biphasic or multiphasic extractant before, during, or after a liquid-liquid or solid-liquid extraction process. Further to this, in experiments employing either dilute or concentrated semi-aqueous solutions, light transmission through the fluid as viewed in the Jerguson Gage window changes from a transparent fluid to a translucent fluid with the introduction of CO₂ gas, indicating the development of one or more solvent interphases and the onset of so-called Marangoni-Rayleigh turbulence driven by surface tension and density gradients between the visible interphases (mass transfer interfaces).

In Sun, Z. et al., “Absorption and Desorption of Carbon Dioxide into and from Organic Solvents: Effects of Rayleigh and Marangoni Instability”, Ind. Eng. Chem. Res. 2002, 41, 1905-1913 (Sun et al.), Sun et al. describe interphase surface patterns created by Marangoni-Rayleigh convection (or turbulence) during absorption and desorption of CO₂ into and from several organic solvents. The research of Sun et al. showed that CO₂ absorbing or desorbing from the different organic solvents creates unique high surface area and turbulent roll or polygonal cellular surface structures as evidenced by Schilieren interference pattern imaging. Moreover, Sun et al. showed that CO₂ absorbing into water produced no interfacial turbulence, and the absorption process is laminar and controlled by the liquid-phase resistance according to penetration theory (CO₂ Gas Phase→CO₂-Water Interface→Liquid Water Phase).

Now referring to FIG. 2, experiments were performed by the present inventors using a concentrated aqueous alcoholic (CAA) semi-aqueous solution, comprising 90%:10% IPA:H₂O vol:vol. Compared to the experiments performed under FIGS. 1A and 1B using a DAA, the CAA semi-aqueous solution produced more pronounced (macroscopic) wavy patterns of Marangoni-Rayleigh turbulence over the pressure range 1 atm to 61 atm. Three exemplary CO₂ pressure points of the same ten pressure points tested under FIG. 1A are shown in FIG. 2: 1 atm, 27 atm, and 61 atm. Shown in FIG. 2, the CAA solution expanded from starting Level A (52) at 1 atm to a salted-out Level B (54), exemplarily shown in FIG. 2 at 27 atm. It can also be seen that, like the DAA solution experiment discussed under FIGS. 1A and 1B, the light transmission properties changed from transparent (56) to translucent (58), indicating the onset of Marangoni-Rayleigh turbulence or convection. Moreover, the interphase volume between Level A (52) and Level B (54) at 27 atm was instantly attained at 7 atm (not shown) and is approximately the same as the interphase volume between FIG. 1A Level A (2) and Level B (4) at 54 atm of CO₂. This was not anticipated, but further validated the mechanisms involved in the CO₂ SALLE process. Equal but opposite stoichiometric proportions of IPA (i.e., 10%/90%) and water (i.e., 90%/10%) were used in the two experiments. This indicates that the salting-out process in a semi-aqueous solution having lower water content completes at a lower CO₂ pressure (i.e., lower aqueous CO₂ concentration). Again, referring to FIG. 2, as the CO₂ gas pressure was increased to 61 atm, a saturated liquid CO₂ phase was formed above the salted-out IPA phase with a meniscus at Level C (60). At 61 atm, the excess amount of salted-out IPA, still containing a small amount of water, quickly formed a saturated solution with liquid CO₂ phase. At this point, the entire solution formed elongated Marangoni-Rayleigh wavy patterns (62) which were larger in width and length as compared to the semi-aqueous CAA solution (58) at 27 atm CO₂ gas pressure. The wavy patterns streamed downward. The increase in Marangoni-Rayleigh turbulence was possibly due to a large increase in aqueous CO₂ concentration as the CO₂ atmospheric density increased from 0.058 g/cm³ at 27 atm (58) to 0.787 g/cm³ at 61 atm (62), a 14× increase.

Finally, unique physicochemical changes in semi-aqueous compositions shown and described under FIGS. 1A and 1B, and FIG. 2, commence with the introduction of a small amount of CO₂ under ambient temperature and relatively low CO₂ pressure conditions for both dilute and concentrated semi-aqueous solutions. These physicochemical changes are selectively controlled by regulating dense phase CO₂ pressure—a key CO₂ SALLE process control variable (KPV) among several others to be discussed herein. Besides CO₂ hydration and ionization phenomenon unique to the dense phase CO₂-water system that drives WSWE compound salting-out phenomenon, the CO₂ SALLE process is also enabled by large differences in polar and hydrogen bonding energies (cohesion energy) within the dense phase CO₂-water biphasic system as well as similarities in cohesion energy within the dense phase CO₂-WSWE biphasic system.

In this regard, in Stone, H. W., “Solubility of Water in Liquid Carbon Dioxide”, Ind. Eng. Chem., 1943, 35, 12, pp. 1284-1286 (Stone), Stone experimentally determined the solubility of water (as a solute) in liquid carbon dioxide (as a solvent) at a pressure between 15 atm and 60 atm and a temperature between (minus) −29° C. and 26.6° C. to range between 0.02% (v:v) and 0.10% (v:v). Stone's liquid CO₂-water solubility results comport with the Jerguson Gage observations described under FIGS. 1A and 1B, and FIG. 2, and are due to the large differences between the HSP's for liquid carbon dioxide (δ_{T}—17.9 MPa^{1/2}) and water (δ_{T}—47.8 MPa^{1/2}). As such, the dense phase CO₂-Water solvent system is biphasic. Further to this, exemplary WSWE compounds suitable for use in the present invention are purposely selected, formulated, and employed as semi-aqueous extraction solutions using Hansen Solubility Parameters (HSP): δ_{D} dispersion energy, δ_{P} polar energy, and δ_{H} hydrogen bonding energy. In this regard, WSWE compounds are chosen which exhibit partial or complete miscibility (or emulsifiability) in water, forming a monophasic semi-aqueous solution. Also, critical to the performance of the CO₂ SALLE process, WSWE compounds are chosen that exhibit at least partial miscibility or expandability (i.e., WSWE compound gas expansion using CO₂) in dense phase CO₂ (gas, liquid or supercritical state). This aspect enables co-extraction, desolvation and extract recovery operations of the present invention. As such, CO₂ SALLE solvent systems are monophasic, biphasic, or multiphasic.

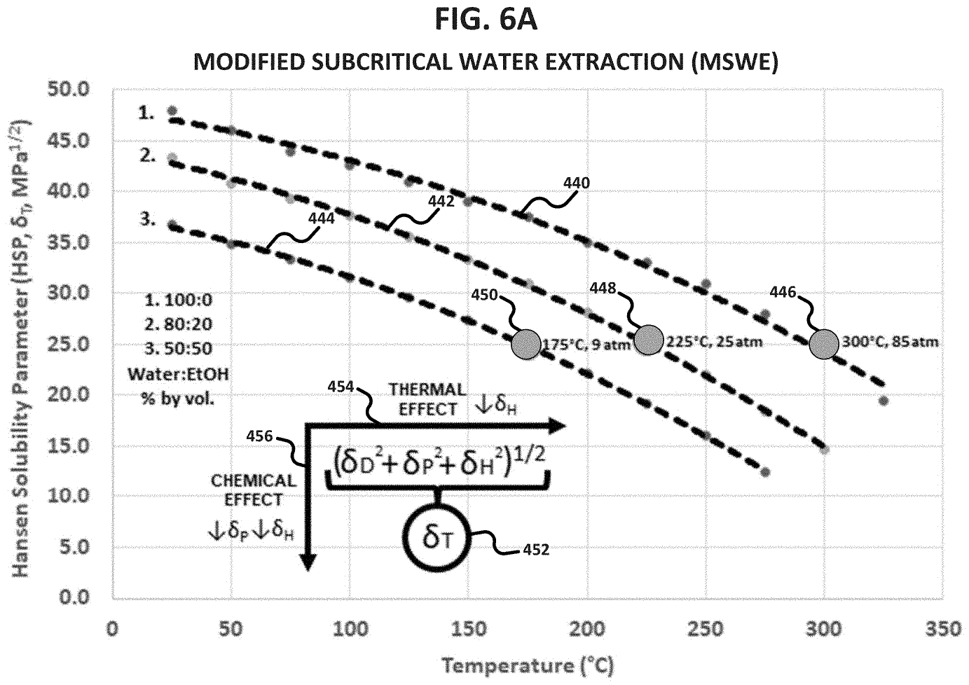

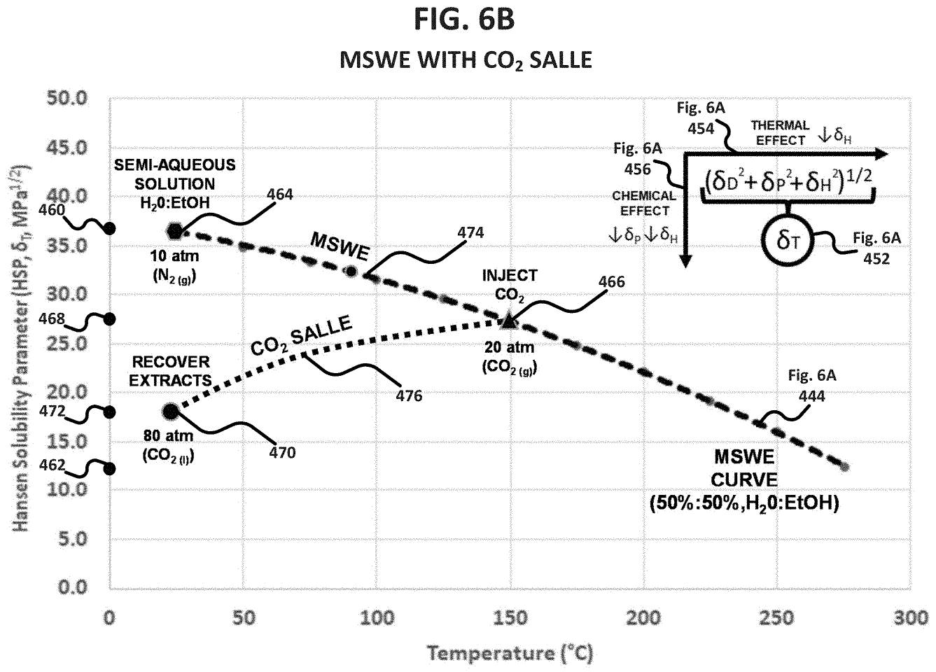

In summary, based on the experimental observations, results, and analysis provided under FIGS. 1A, 1B, 1C, and 1D, and FIG. 2, as well as a comprehensive prior art review, the present inventors believe that the CO₂ SALLE process is a unique development and understanding, particularly as applied to various methods for extracting a substance using a semi-aqueous solvent system (i.e., water is a majority component) and dense phase CO₂, described herein. To support this position, in a comprehensive literature review regarding gas-expanded liquids, and discussed under prior art herein, Jessop and Subramaniam describe a specific phase separation pressure for a water-solvent-CO₂ system above the critical point, for example 77 atm at 40° C. for a water-IPA-CO₂ solvent system. Most significantly, Jessop and Subramaniam do not suggest the unique and pressure-selective CO₂ Gas and Liquid (subcritical CO₂) salting-out phase behavior observed by the present inventors, becoming apparent at pressures as low as 7 atm and at a temperature of 20° C., and which have not been described in the prior art. In this regard, the phenomena observed and described under FIGS. 1A and 1B, and FIG. 2, clearly indicate that both gas-expansion and salting-out phenomena are operating in a water-solvent-CO₂ system. Further to this, these phenomena are controlled by semi-aqueous solution temperature and CO₂ pressure, which controls aqueous CO₂ concentration, as described under FIGS. 1C and 1D. For example, at supercritical temperatures and pressures above the CO₂ critical point (31° C., 73 atm), the salting-out effect is much less prominent than the gas-expansion effect, presumably due to lower aqueous CO₂ solubility and minimal hydrated CO₂ species (i.e., Carbonic Acid) formation until supercritical pressures are reached. At subcritical temperatures below the CO₂ critical temperature and at much lower CO₂ pressures (i.e., visible at 7 atm), salting-out effects dominate. This is evidenced by the significant differences in phase separation behavior observed between dilute water-IPA-CO₂ and concentrated water-IPA-CO₂ solvent systems described in FIGS. 1A and 1B (progressive Level A (2) transition to lower-Level B (4)), and FIG. 2 (instant Level A (52) transition to higher-Level B (54)). Further to this, the discussion under FIGS. 6A and 6B herein describe the unique use of a chemical effect (i.e., WSWE and CO₂ concentrations) and thermal effect (i.e., semi-aqueous solution temperature) as key process variables used to control the CO₂ SALLE process in a water-WSWE-CO₂-substance extraction system, called a “Tunable Extraction System” herein. In this regard, it is hypothesized that CO₂ gas driven expansion of organic compounds dissolved in a semi-aqueous solution, with a subsequent reduction in polar cohesion energy (δ_{P}) of the WSWE solute molecules, in combination with ionized and hydrated CO₂ species (aqueous CO₂ or CO_{2(aq)}), with a subsequent reduction in hydrogen bonding cohesion energy (δ_{H}) on the water molecules, is responsible for the (reversible) phase separation of water-soluble or water-emulsifiable (WSWE) compounds dissolved within a dilute or concentrated semi-aqueous solution. Moreover, the adsorption of CO₂ into a monophasic semi-aqueous solution and the selective emergence of one or more salted-out WSWE compounds, with subsequent adsorption of CO₂ into the newly emergent WSWE phase, is responsible for the formation of biphasic and multiphasic solvent systems exhibiting Marangoni-Rayleigh convection with complex microscopic and macroscopic interfacial pattern formations. Still moreover, the desorption of CO₂ from the dense phase CO₂-Water and dense phase CO₂-WSWE systems reverses WSWE salting-out and solvent expansion effects while sustaining Marangoni-Rayleigh mass transfer enhancement effects.

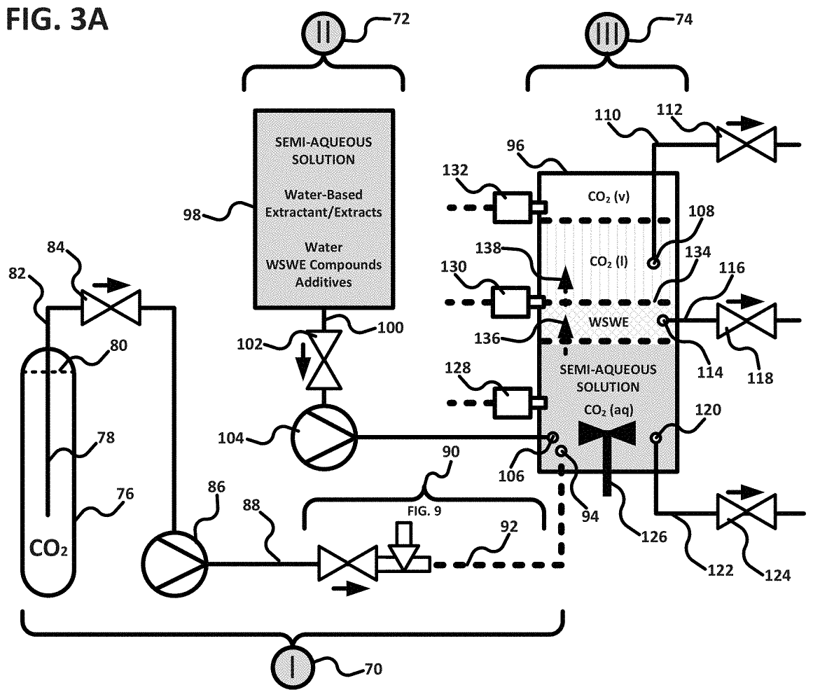

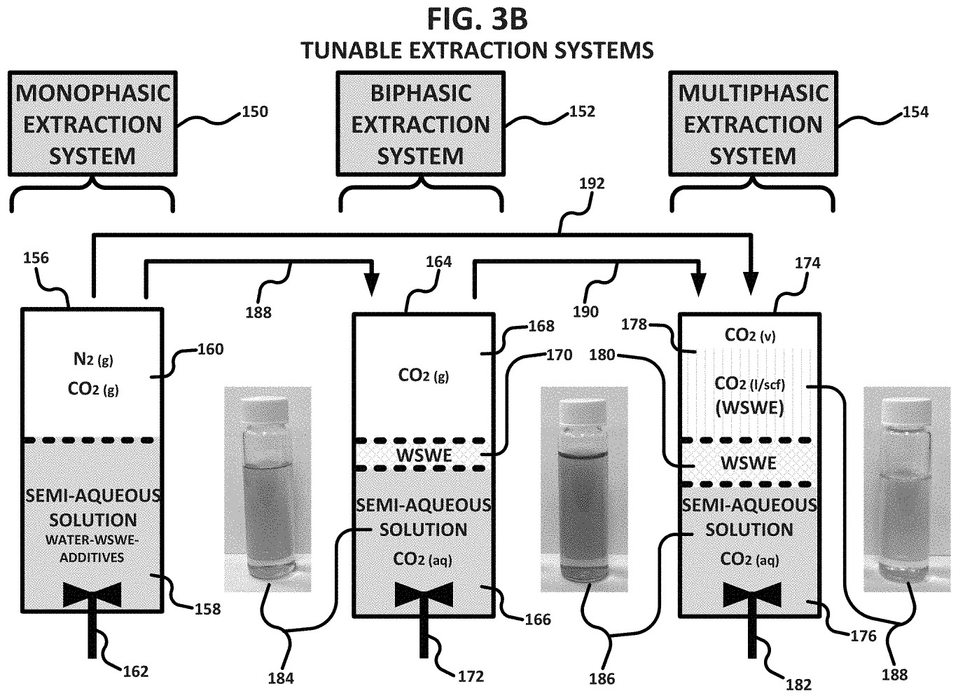

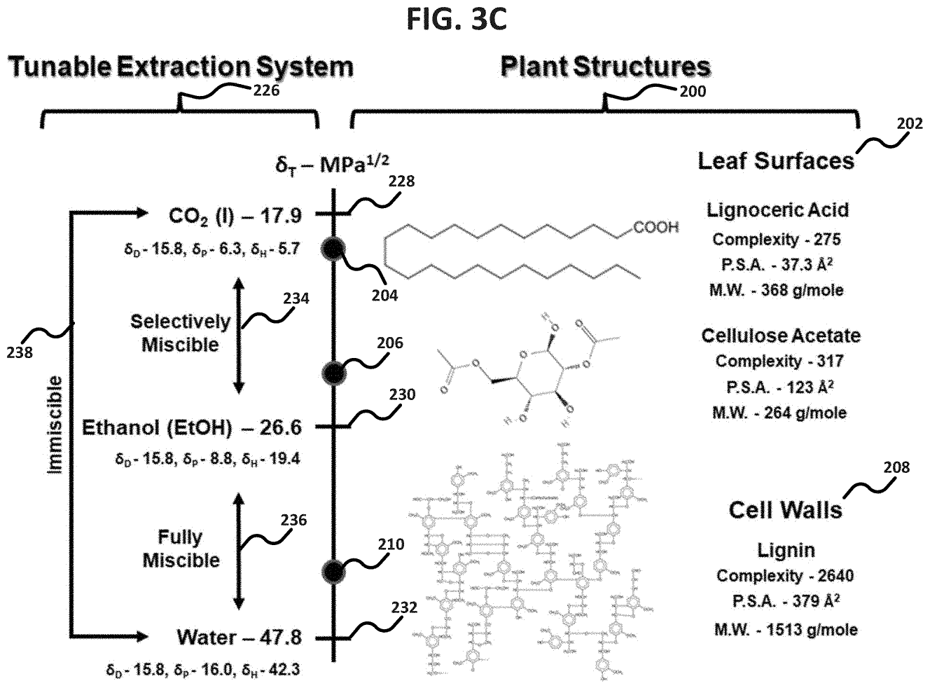

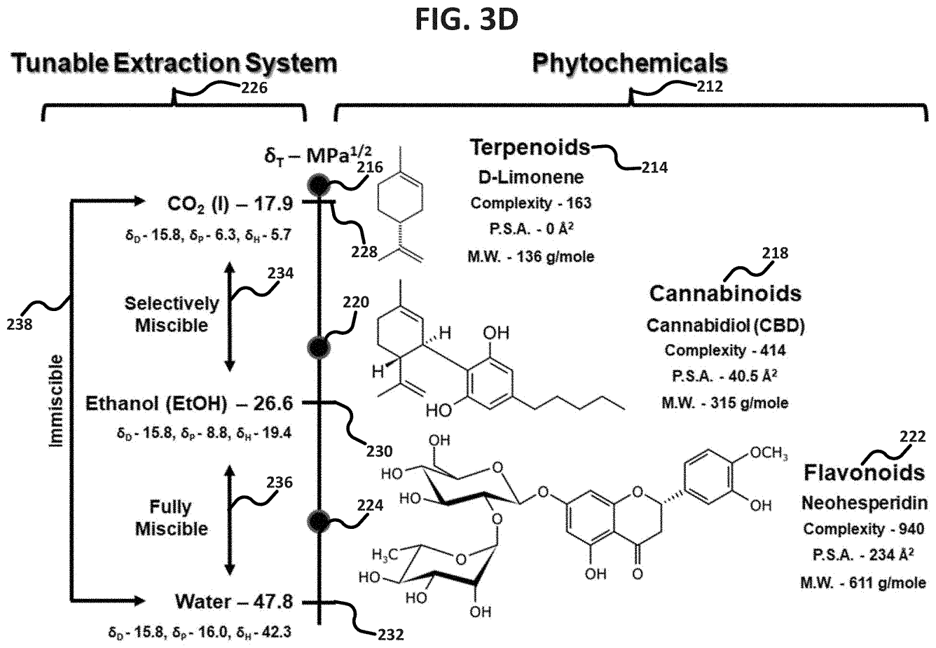

Having described exemplary CO₂ SALLE phenomenon under FIGS. 1A, 1B, 1C, 1D and FIG. 2, the following discussion by reference to FIGS. 3A, 3B, 3C, and 3D describes various aspects of the CO₂ SALLE apparatus and process with an emphasis on the tunable extraction system, cohesion energy characteristics, and relevance to the physicochemistry of the biomaterial system.

FIG. 3A is a schematic describing key aspects of an exemplary CO₂ SALLE apparatus and process of the present invention. Now referring to FIG. 3A, an exemplary CO₂ SALLE apparatus comprises three basic subsystems:
I. Liquid CO₂ Subsystem (70); comprising a Bulk CO₂ Storage Tank, a CO₂ Cylinder, or a Dense Phase CO₂ Recycling System, and a means for transferring and delivering liquid CO₂;
II. Semi-Aqueous Solution Subsystem (72); comprising water containing one or more dissolved water-soluble or water-emulsifiable (WSWE) compounds and optional additives, and heated, unheated, or cooled. The semi-aqueous solution may be a fermented liquid such as an alcoholic beverage containing ethanol and other fermented organic compounds. The semi-aqueous solution may also contain dissolved biomaterial extracts, for example, if previously employed as a water-based extractant (i.e., subcritical water extraction process);
III. CO₂ SALLE Process Vessel Subsystem (74); comprising a pressure vessel suitable for the operating at the temperature and pressure ranges of the present invention, having various inlet and outlet ports for receiving and discharging process fluids such as liquid CO₂, semi-aqueous solution, extracts, and raffinate, and facilitated with various sensors for monitoring temperature, pressure, and semi-aqueous solution level. Further to this, the CO₂ SALLE Process Vessel may contain means for heating or cooling a semi-aqueous solution and biomaterial contained therein, and contain a mixing means for thoroughly mixing solvent phases, or biomaterial and solvent phases, to ensure homogeneity and to enhance interfacial mass transfer prior to phase separation and extract recovery operations of the present invention.

Again, referring to FIG. 3A, the exemplary Liquid CO₂ Subsystem (70) comprises a high pressure CO₂ supply cylinder (76) equipped with an eductor or siphon tube (78) which enables the withdrawal of liquid CO₂ (80) from the CO₂ supply cylinder (76). A liquid CO₂ supply line (82) fluidly interconnects high pressure CO₂ supply cylinder (76), a liquid CO₂ supply valve (84), and a liquid CO₂ supply compressor or pump (86). Finally, said liquid CO₂ supply pump (86) is fluidly interconnected using a high-pressure liquid CO₂ supply line (88) to a CO₂ Aerosol Assembly (90), described in more detail under FIGS. 9A and 9B herein, and fluidly interconnected using one or more flexible polyetheretherketone (PEEK) capillary condensation tubes (92) to one or more bottom-hemisphere located liquid CO₂ inlet ports (94) of the exemplary CO₂ SALLE Process Vessel (96).

Still referring to FIG. 3A, the exemplary Semi-Aqueous Solution Subsystem (72) comprises a semi-aqueous solution holding (and mixing) tank (98), which may contain a heating or cooling means (not shown). Moreover, the exemplary semi-aqueous solution holding tank (98) may contain a mixing means (not shown) for assisting with blending one or more WSWE compounds and optional additives into water to formulate a suitable semi-aqueous solution for use as a primary biomaterial extractant in a liquid-liquid or solid-liquid extraction process. Alternatively, said semi-aqueous solution may be received from a separate water-based extraction process, for example a subcritical water extraction process, whereupon WSWE compounds and optional additives may be added if not already present. Said semi-aqueous solution holding tank, containing a semi-aqueous solution, is fluidly interconnected using a semi-aqueous solution transfer line (100) to a semi-aqueous solution transfer valve (102), semi-aqueous solution transfer pump (104), and semi-aqueous solution inlet port (106) into said exemplary CO₂ SALLE process vessel (96).

Referring to FIG. 3A, the exemplary CO₂ SALLE Process Vessel Subsystem (74) comprises a CO₂ SALLE pressure vessel (96) rated for the operating temperatures and pressures used in the present invention and having various inlet and outlet ports for receiving liquid CO₂ and semi-aqueous solution and for removing CO₂ salted-out solvent-extract mixtures and raffinate. Inlet ports are principally located in the lower hemisphere of the exemplary CO₂ SALLE pressure vessel (96) and comprise one of more liquid CO₂ inlet ports (94) and a semi-aqueous solution inlet port (106). Outlet ports are generally located along a vertical axis ranging from an upper hemisphere to a lower hemisphere of the exemplary CO₂ SALLE pressure vessel (96) based on the presence of a particular solvent phase during phase separation operations of the present invention. Outlet ports comprise one or more Dense Phase CO₂-Extract outlet ports (108) fluidly interconnected using one or more Dense Phase CO₂-Extract transfer lines (110) to one or more Dense Phase CO₂-Extract outlet valves (112). Outlet ports may also comprise one of more salted-out WSWE-Extract outlet ports (114) fluidly interconnected using one or more WSWE-Extract transfer lines (116) to one or more WSWE-Extract outlet valves (118). Finally, outlet ports include one or more Raffinate outlet ports (120) fluidly interconnected using one or more Raffinate transfer lines (122) to one or more Raffinate outlet valves (124). Also shown in FIG. 3A, the exemplary CO₂ SALLE process vessel (96) preferably contains a liquid-liquid and solid-liquid mixing means (126), comprising for example a mixing blade, ultrasonic homogenizer, and/or a centrifuge drum, all of which enabled by conventional external drive and control systems not shown in FIG. 3A. Moreover, electronic or mechanical sensors are used to monitor various physical conditions of the CO₂ SALLE process. These include one or more solvent phase temperature sensors (128), one or more solvent phase level sensors (130), and a pressure sensor (132). The solvent phase level sensors (130) are used to determine the initial semi-aqueous solution level (134), prior to CO₂-salting out operations which enables the emergence of the WSWE-extracts (136) from the semi-aqueous solution and subsequent selective solvation of WSWE-extracts (138) into dense phase CO₂ (forming a miscella), for example a liquid carbon dioxide phase as shown in FIG. 3A. Still moreover, and not shown in FIG. 3A, the exemplary CO₂ SALLE process vessel (96) may be integrated with an analytical chemical process means such as high-performance liquid chromatography (HPLC) or light-induced fluorescence (LIF) spectroscopy to identify and quantify biomaterial extracts, or an electronic density measurement means to analyze changes in the semi-aqueous solution density during CO₂-enabled salting-out and extract recovery operations. Moreover, the various sensors, analytical chemical process means, inlet and outlet valves, and transfer pumps are integrated with a process logic controller (PLC) and software system to execute process fluid transfers, level detection, solid-liquid mixing, phase separations, chemical analysis, extract and process fluid recovery, and raffinate disposal or recycling operations of the CO₂ SALLE process discussed herein.

Still moreover, said CO₂ SALLE pressure vessel (96) may contain a quick-opening closure (not shown) for conveniently introducing and removing a solid material, for example biomaterials contained in a semi-permeable bag, cellulose or glass thimble, or basket, and used to perform in-situ and simultaneous solid-liquid extraction plus CO₂ SALLE extract concentration, desolvation, and recovery processes of the present invention.

Finally, the exemplary CO₂ SALLE apparatus described under FIG. 3A can be operated as a stand-alone primary extraction/co-extraction/extract recovery system (“stand-alone extractor”) for processing a liquid or solid material; or operated as an adjunct co-extraction/extract recovery system (“adjunct extractor”) for processing a water-based extractant derived from a separate primary extraction process, for example hot effluents received from a subcritical water extraction process. A stand-alone extractor performs a primary extraction of a liquid or solid material within a semi-aqueous or non-aqueous phase and is then followed by a CO₂ SALLE co-extraction and extract concentration, desolvation, and recovery operation. An adjunct extractor receives a water-based extractant containing extracts (with or without dissolved WSWE compounds and optional additives) and is processed using exemplary CO₂ SALLE processes herein to concentrate, desolvate, and recover extracts and process fluids.

Operational aspects of the exemplary CO₂ SALLE apparatus and process described in FIG. 3A will be better understood by the following discussion with reference to FIG. 3B. FIG. 3B is a diagram describing tunable solvent phase and solubility properties (i.e., tunable extraction systems) of the present invention as used in a liquid-liquid or solid-liquid extraction process.

Now referring to FIG. 3B, the present invention is a tunable extraction system that provides optimal and full spectrum solvency for nonpolar, polar, and ionic extracts. Liquid-liquid and solid-liquid extraction processes performed using the exemplary apparatus of FIG. 3A comprise one or a combination of tunable extraction systems: monophasic extraction system (150), biphasic extraction system (152), and multiphasic extraction system (154).

A monophasic extraction system (156) employs a semi-aqueous solution (158), containing for example water, one or more WSWE compounds, and optional additives, in a nitrogen (N_{2(g)}) or a CO_{2 (g)} atmosphere (160). The monophasic extraction system (156) is operated at an exemplary semi-aqueous solution temperature between 30° C. and 300° C. and an exemplary N_{2 (g)} or CO_{2 (g)} pressure between 5 atm and 85 atm. N_{2 (g)} pressure is used to provide an inert vapor pressure at elevated temperatures to prevent solution boiling. Moreover, N_{2 (g)} does not expand dissolved WSWE compounds (if present) and does not produce aqueous species in water. As such, N_{2 (g)} is used in a WSWE-modified subcritical water extraction process to provide a monophasic WSWE-infused extraction chemistry. By contrast, CO_{2 (g)} is used in several different ways: 1) provides a vapor pressure to prevent solution boiling, 2) lowers the pH of a semi-aqueous solution (even at low CO₂ pressures (concentrations), and 3) selectively produces biphasic and multiphasic semi-aqueous extraction solutions. The monophasic extraction system (156) of the present invention is essentially a heated pressurized water or modified subcritical water extraction (MSWE) system, which produces a water-based extractant that is further processed using the CO₂ SALLE process to concentrate, desolvate, and recover dissolved extracts contained therein. The MSWE system provides a monophasic extraction solvent system with a Hansen Solubility Parameter (HSP) ranging between about 47.8 MPa^{1/2} and 25 MPa^{1/2}, depending upon the temperature and composition of the semi-aqueous solution. Finally, the MSWE system is preferably a mixed (intensified) system (162) comprising, for example, a mixing blade, ultrasonic homogenizer, or centrifuge drum. A mixing means (162) is preferably employed during a liquid-liquid or solid-liquid extraction process to enhance mass transfer.

Still referring to FIG. 3B, a biphasic extraction system (164) employs a semi-aqueous solution (166), containing for example water, one or more WSWE compounds, and optional additives, in a dense phase CO_{2 (g)} atmosphere (168). The biphasic extraction system (164) is operated at an exemplary semi-aqueous solution temperature between −40° C. and 50° C. and an exemplary CO_{2 (g)} pressure between 5 atm and 50 atm, which selectively produces (based on CO₂ pressure and semi-aqueous solution temperature) a CO₂ salted-out WSWE compound mixture phase (170). The biphasic extraction system (164) of the present invention provides a semi-aqueous extraction solvent phase (Phase 1) with a Hansen Solubility Parameter (HSP) ranging between about 47.8 MPa^{1/2} and 35 MPa^{1/2}, depending upon the CO₂ gas pressure, and temperature and composition of the semi-aqueous solution. Moreover, the biphasic extraction system produces a non-aqueous CO₂ salted-out WSWE compound mixture phase (Phase 2) with a Hansen Solubility Parameter (HSP) ranging between 20 MPa^{1/2} and 30 MPa^{1/2}, depending upon the composition and temperature of the CO₂ salted-out WSWE compound mixture. Finally, the biphasic system preferably employs a mixing means (172) to blend the biphasic system during liquid-liquid or solid-liquid extraction processes to intensify mass transfer. This is accomplished using for example a mixing blade, ultrasonic homogenizer, or centrifuge drum. Following a mixed biphasic extraction process, the mixing operation is halted to allow the biphasic system to stratify into discrete phases along a vertical axis in preparation for extract concentration, desolvation, and recovery operations.

Still referring to FIG. 3B, a multiphasic extraction system (154) employs a semi-aqueous solution (176), containing for example water, one or more WSWE compounds, and optional additives, as a co-extractant in a dense phase CO₂ liquid or supercritical solvent (178). The multiphasic extraction system (164) is operated at an exemplary semi-aqueous solution temperature between −40° C. and 60° C. and an exemplary dense phase CO₂ pressure between 65 atm and 100 atm, which selectively produces (based on CO₂ pressure and semi-aqueous solution temperature) a non-aqueous dense phase CO₂ liquid or supercritical fluid as a upper phase (178), a non-aqueous CO₂ salted-out WSWE compound mixture as a middle phase (180), and a semi-aqueous solution as a lower phase (176). Given this, the multiphasic extraction system (174) of the present invention provides a lower semi-aqueous extraction solvent phase (Phase 1) with a Hansen Solubility Parameter (HSP) ranging between about 47.8 MPa^{1/2} and 35 MPa^{1/2}, depending upon the CO₂ gas pressure, and temperature and composition of the semi-aqueous solution. Moreover, the multiphasic extraction system produces a middle non-aqueous CO₂ salted-out WSWE compound mixture phase (Phase 2) with a Hansen Solubility Parameter (HSP) ranging between 20 MPa^{1/2} and 30 MPa^{1/2}, depending upon the composition and temperature of the CO₂ salted-out WSWE compound mixture. Still moreover, the multiphasic extraction system produces an upper non-aqueous dense phase CO₂ phase comprising liquid or supercritical CO₂ (Phase 3) with a Hansen Solubility Parameter (HSP) ranging between 12 MPa^{1/2} and 20 MPa^{1/2}, depending upon the composition and temperature of the CO₂ salted-out WSWE compound mixture. Finally, the multiphasic system preferably employs a mixing means (182) to blend the multiphasic system during liquid-liquid or solid-liquid extraction processes to intensify mass transfer. This is accomplished using, for example, a mixing blade, ultrasonic homogenizer, or centrifuge drum. Following a mixed multiphasic extraction process, the mixing operation is halted to allow the multiphasic system to stratify into discrete phases along a vertical axis in preparation for extract concentration, desolvation, and recovery operations.

Finally, with reference to FIG. 3A and FIG. 3B, the following discussion provides an exemplary application and operation of the CO₂ SALLE apparatus and tunable solvent system using an alcoholic beverage as a semi-aqueous solution. In this example, a tincture comprising fermented ethanol and ethanol-soluble whiskey organic compounds is selectively salted-out, concentrated, and desolvated at a temperature between 0° C. and 20° C. and a dense phase CO₂ pressure of between 15 atm and 80 atm. Now referring to FIG. 3A, Bourbon Whiskey (80 Proof) is poured into the semi-aqueous solution storage container (FIG. 3A (98)). Following this, the whiskey is transferred through semi-aqueous solution transfer line (FIG. 3A, (100)), opened transfer valve (FIG. 3A, (102)), and using transfer pump (FIG. 3A, (104)) to fill the CO₂ SALLE process vessel (FIG. 1A, (96)) until the fill level sensor (FIG. 3A, (130)) is triggered. Following this, solution transfer valve (FIG. 3A, (102)) is closed and solution transfer pump (FIG. 3A, (104)) is stopped. Following the transfer of a quantity of whiskey into the CO₂ SALLE process vessel (FIG. 3A, (96)), the CO₂-extract valve (FIG. 3A, (112)) is opened to allow the CO₂ SALLE process vessel (FIG. 3A, (96)) to vent to atmosphere during a CO₂ cool down and CO₂ saturation process. In this regard, a CO₂ aerosol injector assembly (FIG. 3A, (90)) is used to inject a near-cryogenic CO₂ solid-gas particle stream through CO₂ inlet port (FIG. 3A, (94)) and into the whiskey solution contained in the CO₂ SALLE process vessel (FIG. 3A, (96)). This is performed using a supply of liquid CO₂ (FIG. 3A, (76)) fluidly interconnected through liquid CO₂ supply line (82), opened liquid CO₂ supply valve (FIG. 3A, (84)), through (de-energized) liquid CO₂ pump (FIG. 3A, (86)), high pressure liquid CO₂ supply line (FIG. 3A, and into said CO₂ aerosol injector assembly (FIG. 3A, (90)). During, whiskey cool down and CO₂ saturation to a temperature between 0° C. and 20° C., the CO₂ SALLE process vessel (FIG. 3A, (96)) vents CO₂ gas to the atmosphere through opened CO₂-extract valve (FIG. 3A, (112)). Once the desired temperature is reached, as determined by a temperature sensor (FIG. 3A, (128)), the CO₂-extract valve (FIG. 3A, (112)) is closed while continuing to inject CO_{2 (s-g)} aerosol into the whiskey. During this step, the cold whiskey will pressurize to between 15 and 50 atm as cold CO₂ flow into the vessel slowly declines. During CO₂ autogenous pressurization, and now referring to FIG. 3B, the cold whiskey solution (FIG. 3B, (184)), shown in a glass vial (184), begins to salt-out the fermented ethanol and ethanol-soluble whiskey organic compounds, becoming darker in color as the salted-out whiskey (FIG. 3B, (186)), also shown in a glass vial (186), loses a portion of its ethanol and ethanol-soluble organic compound content as a CO₂ salted-out whiskey tincture (FIG. 3B, (170)). Finally, the liquid CO₂ pump (FIG. 3A, (86)) is energized and the CO₂ SALLE process vessel (FIG. 3A, (96)) is pressurized to CO₂ saturated liquid conditions, between 60 atm and 80 atm, as determined by a pressure sensor (FIG. 3A, (132)). Following this, the CO₂ pump is de-energized, and the CO₂-extract valve (FIG. 3A, (112) is opened, and the CO₂-extract phase (FIG. 3B, (178)) is withdrawn and desolvated to form a whiskey flavor-infused tincture (FIG. 3B, (188)), shown in a glass vial (188). The ethanol-rich whiskey flavor-infused tincture has a taste that is similar to the 80 Proof Bourbon Whiskey (starting solution) but has a lighter color. The much darker extracted whiskey (raffinate) solution has a much lighter whiskey flavor and odor as compared to the starting solution but has a much darker color. This indicates an increased concentration of water-soluble pigments and selectivity of the CO₂-salted out WSWE mixture. Finally, using the exemplary semi-aqueous whiskey solution in a solid-liquid extraction process, for example co-extracting ground cannabis plant located within the dense CO₂ phase (not shown), a whiskey flavor-infused cannabis extract tincture can be produced.

Looking at FIG. 3B, the tunable monophasic (150), biphasic (152), and multiphasic (154) extraction systems of the present invention may be used individually or sequentially, and reversibly. For example, the monophasic extraction (156) may be followed (188) by a biphasic extraction (164), and then followed (190) by a final multiphasic extraction process, and completed with CO₂ SALLE extract concentration, desolvation, and recovery operations described herein. In another example, the monophasic extraction (156) may be followed (192) by a multiphasic extraction (174). Moreover, the exemplary sequencing thus described it is reversible. This extraction solvent sequencing is called solvent phase shifting and produces full-spectrum biomaterial extracts when used in a solid-liquid extraction process.

An exemplary semi-aqueous extraction method for forming an alcoholic mixture containing an extract comprises: a semi-aqueous extraction method for forming an alcoholic mixture, the steps comprising:
1. Placing a natural product containing an extract into a pressure vessel (FIG. 3A, (74));
2. Adding an alcoholic beverage containing fermented ethanol and ethanol-soluble fermented compounds to the pressure vessel (FIG. 3A, (72));
a. Pressurizing said alcoholic beverage and the natural product using dense phase CO₂ (FIG. 3A, (70)) to establish a tunable extraction system in the pressure vessel (FIG. 3B, (150));
3. Expanding and salting-out said tunable extraction system using said dense phase CO₂ to produce a first separated phase, which comprises fermented ethanol, ethanol-soluble fermented compounds, and the extract (FIG. 3B, (152));
4. Simultaneously co-extracting said first separated phase using said dense phase CO₂ to produce a second separated phase, which comprises a CO₂ salted-out solvent mixture containing the fermented ethanol, the ethanol-soluble fermented compounds, and the extract (FIG. 3B, (154)); and
5. Desolvating said CO₂ salted-out solvent mixture to concentrate and to form the alcoholic mixture (FIG. 3A, (74)).

Wherein said alcoholic beverage comprises beer, vodka, port, rum, gin, whiskey, bourbon, brandy, grain alcohol, cognac, tequila, wine, baijiu, sake, soju, hard seltzer, or hard cider; and said alcoholic mixture is desolvated to form a non-alcoholic concentrate.

The alcoholic mixture may be desolvated using, for example, vacuum distillation to remove fermented ethanol, which leaves a healthy and flavorful non-alcoholic beverage extract or concentrate. The non-alcoholic beverage extract can be added directly to foods and beverages or formulated into an emulsion to form a water-soluble composition.

In summary, the monophasic, biphasic, and multiphasic CO₂ SALLE process used in a tunable extraction system as described under FIGS. 3A and 3B comprises the following exemplary method:

A semi-aqueous extraction method for recovering an extract from a substance, the steps comprising:
1. Placing the substance into a pressure vessel (FIG. 3A, (74));
2. Adding a semi-aqueous solution, comprising a mixture of water and water-soluble or water-emulsifiable compound, to the pressure vessel (FIG. 3A, (72));
3. Pressurizing said semi-aqueous solution and the substance using dense phase CO₂ (FIG. 3A, (70)) to establish a tunable extraction system in the pressure vessel (FIG. 3B, (150));
4. Expanding and salting-out said tunable extraction system using said dense phase CO₂ to produce a first separated phase, which comprises the water-soluble or water-emulsifiable compound containing the extract (FIG. 3B, (152)); and
5. Simultaneously co-extracting said first separated phase into said dense phase CO₂ to produce a second separated phase, which comprises a CO₂ salted-out solvent mixture containing the extract (FIG. 3B, (154)).

Wherein said substance comprises natural product, pomace, animal tissue, soil, sludge, slurry, potable water, alcoholic beverage, fermentation broth, industrial wastewater, fermented food, or water-based extractant; said extract comprises phytochemical, essential oil, polyphenol, fermented compound, fermented ethanol, ethanol-soluble compound, decarboxylated compound, psychoactive compound, terpenoid, cannabinoid, flavonoid, carboxylic acid, protein, oxygenated compound, organic compound, metalorganic compound, inorganic compound, chemical pollutant, or ionic compound; said water-soluble or water-emulsifiable compound comprises alcohol, polyol, ketone, ester, nitrile, ether, organosulfur compound, surfactant, emulsion, hydrotrope, or aqueous carbon dioxide; said dense phase CO₂ comprises gaseous CO₂, solid CO₂, liquid CO₂, or supercritical CO₂; said dense phase CO₂ is contacted with said tunable extraction system at a temperature between −40° C. and 300° C. and at a pressure between 1 atm and 340 atm; said dense phase CO₂ is preferably contacted with said tunable extraction system at a temperature between −20° C. and 150° C. and a pressure between 5 atm and 150 atm; said CO₂ salted-out solvent mixture comprises gaseous CO₂ and CO₂ expanded and salted-out water-soluble or water-emulsifiable compound, liquid CO₂ and CO₂ expanded and salted-out water-soluble or water-emulsifiable compound, or supercritical CO₂ and CO₂ expanded and salted-out water-soluble or water-emulsifiable compound; said CO₂ salted-out solvent mixture is a water-soluble or water-emulsifiable-rich CO₂ salted-out solvent mixture containing the extract and a dense phase CO₂-rich CO₂ salted-out solvent mixture containing the extract; a quantity and Hansen Solubility Parameters of said water-soluble or water-emulsifiable compound contained in said tunable extraction system are calculated based on an amount and Hansen Solubility Parameters of the extract to be extracted by said water-soluble or water-emulsifiable compound; a quantity and Hansen Solubility Parameters of said dense phase CO₂ are calculated based on an amount and Hansen Solubility Parameters of said water-soluble or water-emulsifiable compound containing the extract to be co-extracted by said dense phase CO₂; said tunable extraction system is mixed with additives comprising purified water, organic acid, organic salt, inorganic salt, surfactant, co-surfactant, enzyme, pH buffer, chelation agent, triacetin, or ozone; said water-soluble or water-emulsifiable compound contained in said tunable extraction system is selectively expanded and salted-out using CO₂ pressure, CO₂ temperature, and CO₂ volume; a concentration of said water-soluble or water-emulsifiable compound in said tunable extraction system or said CO₂ salted-out solvent mixture is between 0.1% and 95% by volume; said CO₂ salted-out solvent mixture is used in a secondary process comprising solid-liquid extraction process, liquid-liquid extraction process, analytical chemical process, desolvation process, ozonation process, fractionation process, or decarboxylation process; said desolvation process comprises utilizing gravity separation, phase separation, near-cryogenic phase separation, high pressure distillation, atmospheric distillation, vacuum distillation, membrane separation, gas flotation, or evaporation to form a desolvated CO₂ salted-out solvent mixture, which comprises a water-soluble or water-emulsifiable compound containing the extract; an ozonated gas is bubbled through said desolvated CO₂ salted-out solvent mixture to form an oxygenated extract; said ozonated gas has a concentration between 0.2 mg/hour and 15000 mg/hour of ozone gas at a temperature between minus 20 degrees C. and 30 degrees C., and a pressure of about 1 atm; the concentration of said oxygenated extract is monitored and controlled using a digital timer or a viscosity sensor; said analytical chemical process comprises analyzing the extract dissolved in said CO₂ salted-out solvent mixture using UV-VIS spectrophotometry, fluorescence spectroscopy, Raman spectroscopy, gas chromatography, high-performance liquid chromatography, ion chromatography, liquid density analysis, or gravimetric analysis; and Said analytical chemical process is performed in-situ or ex-situ.

Having described the exemplary apparatus and tunable extraction system under FIGS. 3A and 3B, following is a detailed discussion of a fundamental application for the present invention, plant and phytochemical extraction, by reference to relevant literature research and FIGS. 3C and 3D.

The present invention is useful in a variety of liquid-liquid and solid-liquid extraction applications. However, biomaterials such as herbs and spices present a unique set of solvent extraction process challenges. Example challenges include extraction solvent access to plant materials, extraction solvent solubility characteristics, and mass transfer characteristics for the vast range of plants and phytochemicals. A particular herb or spice contains a significant variety of phytochemicals. These phytochemicals possess different polarities, densities, molecular structures and complexities, molecular weights, states of matter (liquid or solid), and concentration. Moreover, phytochemicals are located and concentrated in different locations and structures of the plant, for example leaves, bark, membranes, roots, seeds, and flowers. In some extraction applications, for example cannabis and hemp, target phytochemicals such as terpenoids and cannabinoids are concentrated in glandular structures called trichomes, which are located on the leaves and flowers of these plant systems. In this regard, hemp and cannabis extractions are straightforward using a monophasic solvent system such as hexane, carbon dioxide, or ethanol, among many other solvents. However, other types of herb and spice extraction applications involve phytochemicals such as highly polar polyphenols which are located inside cellular structures encased by cutaneous, cellulosic, and other water-bearing structures, for example as present in fruit and vegetable pomaces. These water-bearing structures are barriers to mass transfer. Extraction and recovery of these types of phytochemicals is much more challenging and requires longer processing times, higher processing temperatures, and newer tunable solvent extraction processes such as subcritical water extraction. Given this, and as discussed herein, the present invention provides a tunable extraction system, and is particularly directed to biomaterial extraction applications involving substances such as herbs, spices, pomaces, among many other botanical examples.

A key process variable in botanical extractions is the optimization of both solvent penetration into plant structures, and solvation of organic compounds contained within these structures (i.e., solvent cohesion energy (solubility) characteristics and temperature). If the target compound (i.e., lycopene) is contained within a plant structure (i.e., tomato skin), mixed-polarity solvent blends are needed for swelling the plant structure to improve both solvent penetration and extract solvation processes. This is best understood by the following discussion regarding the physicochemical characteristics of plant surfaces and solvent blends used to optimize extraction of organic components from same.

According to Khayet, M. et al., “Estimation of the Solubility Parameters of Model Plant Surfaces and Agrochemicals: A Valuable Tool for Understanding Plant Surface Interactions”, Theoretical Biology and Medical Modelling 2012, 9:45 and Khayet, M. et al., “Evaluation of the Surface Free Energy of Plant Surfaces: Toward Standardizing the Procedure”, Frontiers in Plant Science, 1 Jul. 2015, Volume 6, Article 510 (Khayet et. al.), plant surfaces are a complex system. For example, the cuticle is made of a bio-polymer matrix, waxes that are deposited on to (epicuticular) or intruded into (intracuticular) this matrix, and variable amounts of polysaccharides and phenolics. Waxes commonly constitute 20 to 60% of the cuticle mass and are complex mixtures of straight chain aliphatics. The cuticle matrix is commonly made of cutin, which is a biopolymer formed by a network of inter-esterified, hydroxyl- and hydroxy-epoxy C16 and/or C18 fatty acids. Further to this, the cuticle acts as a “solution-diffusion” membrane for the diffusion of some solvents and solutes.

The total surface free energies of plant surfaces are diverse. For example, peach and pepper fruits have similar surface free energies (SFE), approximately 32.2 mN/m, but are significantly higher than that measured for Eucalyptus leaves, 17.4 mN/m. Concerning solubility parameters, Eucalyptus leaves exhibit a significantly lower value, 10.6 MPa^{1/2}, than pepper and peach fruit surfaces, 17 MPa^{1/2}. The dominant class of compounds in both pepper and peach fruit waxes is n-alkanes, which have a solubility parameter around 16 MPa^{1/2} for the most abundant compounds reported (C23 to C31 n-alkanes).

Given this, it is understood that the botanical system represents a complex extraction environment, with variable plant substances and surfaces having different SFE and solubility parameters. Moreover, according to Khayet et al., a solubility parameter gradient is established from the external and more hydrophobic epicuticular wax layer towards the more hydrophilic internal cell wall. Owing to the properties of the dominant epicuticular waxes present in the analyzed plant materials, it is concluded that the solubility parameter increases with increasing depth from the epicuticular wax surface towards the internal cell wall.

In this regard, it is understood that an optimal solvent chemistry is necessary, as well as mechanical and thermal optimizations, which provides both polar and nonpolar cohesion energies necessary to extract nonpolar lycopene located within polar cellulosic tissues of plants (i.e., tomato skins). Swelling the cellulosic structures is an important process variable during solvent extraction. A mixed-polarity solvent is required to provide cellulosic swelling, solvent penetration, and solvation of lycopene. As such, homogeneous solvent mixtures should be used that exhibit two distinct properties: (a) high lycopene affinity and (b) ability to swell the plant material and thus enhance solvent penetration and solvation phenomenon.

According to Zuorro, A., “Enhanced Lycopene Extraction from Tomato Peels by Optimized Mixed-Polarity Solvent Mixtures”, Molecules 2020, 25, 2038 (Zuorro), cellulose is organized in microfibrils containing both crystalline and amorphous regions. Microfibrils are assembled into fibers of larger diameter that are cross-linked by hemicelluloses and embedded in a gel-like pectic matrix. The degree of cellulose crystallinity and the spatial organization of the cellulose/hemicellulose network are mainly determined by intra- and intermolecular hydrogen bonds, formed between hydroxyl groups present in the β-1,4-linked D-glucopyranose units of cellulose. Solvent molecules of small size and high polarity can penetrate the plant matrix and adsorb on these hydroxyl groups. Following adsorption, some bonds are broken, increasing the distance between the cellulose fibers, and causing the material to swell. In most cases, swelling is limited to the amorphous regions of cellulose, which are more reactive and accessible to solvent. Moreover, a multi-polar blended solvent system is best for extracting lycopene from tomato pomace. Conventionally, a hexane-ethanol-acetone blend provides optimum extraction efficiency. However, tests substituting ethyl lactate, also an excellent solvent for lycopene, for the hexane component of the solvent blend produces inferior extraction efficiency. The cause for this is attributed to solvent complexation between ethyl lactate and ethanol molecules, resulting in reduced plant tissue swelling.

As such, an important aspect of the present invention is that dense phase CO₂ behaves as a penetrant and plasticizer for polymeric matrices. This beneficial characteristic is related to liquid phase organic solvent expansion effects and is well established in the prior art for many different solid phase organic polymers. For example, in Sawan, S. P. et al., “Evaluation of Interactions Between Supercritical Carbon Dioxide and Polymer Materials”, Los Alamos National Laboratory, Report LA-UR-94-2341, 1994 (Sawan), Sawan states that high pressure carbon dioxide can cause absorption, swelling, and solvation of some polymers as evidenced by weight change data from treatments in dense phase carbon dioxide (liquid and supercritical). Amorphous polymers such as PMMA, PETG, ABS, CAB, and HIPS show more significant absorption, swelling and solvation than crystalline polymers. Moreover, Sawan emphasizes that dense phase carbon dioxide plasticizes most polymers and can cause a significant reduction in glass transition temperature (Tg). Given this, dense phase CO₂ used as a component in aqueous solvent blends assists with solvent penetration and solvation of organic extracts contained within cellulosic plant structures. For example, the ethyl lactate-ethanol complexation constraint described by Zuorro can be mitigated using, for example, an expanding and salting-out solvent blend comprising dense phase CO₂-ethyl lactate-water.

Biomaterials such as herbs and spices provide a very diverse and complex mixture of hundreds of potentially extractable organic and organometallic chemistries (i.e., phytochemicals) ranging from nonpolar to highly polar compounds; with straight chain to highly branched, to multi-cyclic chemical structures; and exhibiting volatility or non-volatility. All of this is further complicated by physical aspects and properties of the botanical solid substance, for example plant cellulosic structures and plant cellular membrane barriers. As such, many factors must be considered to optimize a biomaterial extraction process. Key process variables (KPVs) include:
Extract cohesion chemistry (i.e., dispersive, polar, and hydrogen bonding energies);
Cohesion chemistry of physical structures (i.e., vacuoles, cell walls, membranes, tissues, and organs);
Moisture content;
Botanical material pretreatments such as drying and grinding;
Cohesion chemistry of extraction solvent or solvent blend;
Extraction solvent-solid volume-mass ratio;
Extraction solvent temperature and pressure;
Extraction process intensification energies such as ultrasonics, microwaves, and centrifugation;
Extraction (solvent-substance contact) time;
Extract concentration (change over time); and
Extract recovery process (i.e., prevent degradation or volatile losses).

Given this, there is no one universal extraction solvent, or one best extraction technique, to perfectly address each of these KPVs. In this regard, the tunable extraction system of the present invention provides a more robust exhaustive extraction process as compared to a conventional so-called tunable solvent system. For example, in U.S. Pat. Nos. '366 and '112 by the first-named inventor of the present invention, the cohesion properties of dense phase CO₂ are adjusted using pressure and temperature, and using organic solvent pre-treatments and modifiers. These conventional tunable solvent systems are also used with process intensification techniques such as phase shifting and centrifugation. The commercial application of a conventional tunable solvent system is detailed by the first-named inventor in Jackson, D., “CO₂ for Complex Cleaning”, Process Cleaning Magazine, July/August 2009 (Jackson).

In contrast with tunable solvent systems, the present invention uniquely combines the tunable solvent properties of a non-aqueous dense phase CO₂ extraction system and a semi-aqueous solvent extraction system, working cooperatively as a tunable extraction system, to optimize the extraction of organic, inorganic, and ionic compounds from one or a combination of solid and/or liquid substances. The present invention enables in-situ formation and use of blends of dense phase CO₂, semi-aqueous solvent, and expanded/salted-out WSWE compounds in multiphasic liquid-liquid and solid-liquid extractions. These tuned extraction systems are based on like-dissolves-like (i.e., matching dispersive, polar, and hydrogen bonding energies between extraction solvent environment and substance) and like-seeks-like (i.e., maximizing cellular or cellulosic swelling and penetration) principles of Hansen Solubility Parameters. Tunable monophasic, biphasic, and multiphasic solvent chemistry used in combination with extraction process intensification techniques such as optimized thermal and mechanical energy inputs provide an efficient and full-spectrum extraction and recovery process.

In this regard, it is known in the prior art that utilizing both hydrocarbon-like and water-like cohesion chemistry together in a solvent blend broadens the spectrum of compounds that can be extracted from a botanical compound. For example, hydroethanolic solvents significantly improve the solubility of polar flavonoids, which are bioactive polyphenolic compounds. In Zhang, J. et al., “Solubility of Naringin in Ethanol and Water Mixtures from 283.15 to 318.15 K”, Journal of Molecular Liquids, Volume 203, March 2015, pp. 98-103 (Zhang et al.), it was determined that the hydroethanolic solvent system comprising between 40% and 60% ethanol by volume produced the highest solubility of naringin (from grapefruit peels) between the temperature range of 10° C. to 45° C., with naringin solubility increasing with temperature. In Liu, Y. et al., “Optimization of Extraction Process for Total Polyphenols from Adlay”, European Journal of Food Science and Technology, Vol. 3, No. 4, pp. 52-58, September 2015 (Liu et al.), it was determined that optimal extraction of total polyphenols from botanical solid Adlay (Chinese Barley) occurred with a hydroethanolic solution having 60% (by vol.) ethanol at 40° C. for 1.5 hours. Further to this, the results showed that the impact order of the influence factors was 1. ethanol concentration→2. extraction time→3. extraction temperature. Finally, in de Sousa, C. et al., “Greener Ultrasound-assisted Extraction of Bioactive Phenolic Compounds in Croton heliotropiifolius Kunth leaves”, Microchemical Journal, 159 (2020) 105525 (de Sousa et al.), it was determined that optimal extraction of polyphenolic compounds ranged from 88% to 94% using a hydroethanolic solvent comprising 37.5% (by vol.) ethanol at a temperature of 54.8° C. for 39.5 min in an ultrasonic bath.

Having discussed the relevant literature research supporting the need for a tunable extraction system, following is a discussion of an exemplary tunable extraction system comprising dense phase (gas-liquid) CO₂, ethanol, and water, and by reference to FIGS. 3C and 3D. In this regard, FIG. 3C is a diagram related to FIGS. 3A and 3B describing solubility properties of exemplary plant structures, and FIG. 3D is a diagram related to FIGS. 3A and 3B describing solubility properties of exemplary phytochemicals contained in exemplary plant structures of FIG. 3C.

FIGS. 3C and 3D illustrate the how a tunable extraction system of the present invention optimizes extraction solvent chemistry based physicochemical gradients present in plant structures and phytochemicals.

Now referring to FIG. 3C, plant structures (200) exhibit a total HSP ranging between approximately 16 MPa^{1/2} to 35 MPa^{1/2}, which correlates very well with a physicochemical gradient based upon increasing molecular complexity, polar surface area (P.S.A.), and molecular weight (M.W.). For example, as shown in FIG. 3C, plant leaf surfaces (202) contain a compound called Lignoceric Acid (204) with a molecular complexity of 275, a P.S.A. of 37.3 Å², and a M.W. of 368 g/mole. Further into the plant interior is a prevalent compound called Cellulose Acetate (206) with a molecular complexity of 317, a P.S.A. of 123 Å², and a M.W. of 264 g/mole. Finally, throughout the plant physical structure is a compound that forms cell walls (208) from a compound called Lignin (210), with a molecular complexity of 2640, a P.S.A. of 379 Å², and a M.W. of 1513 g/mole.

Now referring to FIG. 3D, and similar to plant structures (200) discussed under FIG. 3C, phytochemicals (212) contained in plant structures exhibit a physicochemical gradient which manifests as a total HSP ranging between approximately 16 MPa^{1/2} to 35 MPa^{1/2} based upon increasing molecular complexity, polar surface area (P.S.A.), and molecular weight (M.W.). For example, as shown in FIG. 3D, a class of compounds called Terpenoids (214) contain a compound called D-Limonene (216) with a molecular complexity of 163, a P.S.A. of 0 Å², and a M.W. of 136 g/mole. Another class of compounds called Cannabinoids (218) contain a compound called Cannabidiol (220) with a molecular complexity of 414, a P.S.A. of 40.5 Å², and a M.W. of 315 g/mole. Finally, still another class of compounds called Flavonoids (222) contain a compound called Neohesperidin (224) with a molecular complexity of 940, a P.S.A. of 234 Å², and a M.W. of 611 g/mole.

With respect to both plant structures and phytochemicals, increasing molecular complexity, polar surface area, and molecular weight requires increasing levels of extraction process intensification, for example increasing temperature, solvent agitation, solvent exchange, and solvent cohesion energy, to efficiently drive the botanical extraction process. Solid phase plant structures may be waxy, cutaneous, cellulosic, and generally polymeric in nature. This requires a more complex extraction environment operating at higher temperatures to induce swelling or plasticization to improve solvent access and solubilization of liquid or solid phytochemicals contained therein. This aspect is a primary motivation for the present invention.

In this regard, and now referring to both FIG. 3C and FIG. 3D, an exemplary tunable extraction system (226) is dense phase CO₂ (CO₂ (g/l)) with a total HSP (ST) of 17.9 MPa^{1/2} (CO₂ (1), (228)), ethanol (EtOH) with a δ_{T} of 26.6 MPa^{1/2} (230), and water (H₂O) with a δ_{T} of 47.8 MPa^{1/2} (232). Important solubility aspects of the exemplary tunable extraction system (226) are that EtOH (230), an exemplary WSWE compound, is selectively and partially soluble (234) in dense phase CO₂ (228), and fully miscible (236) with H₂O (232). Moreover, dense phase CO₂ (1) is practically insoluble (238) in H₂O (232) and dissolves into solution to form hydrated and ionized CO₂ species (CO₂ (aq)), which forms the basis for the monophasic, biphasic, and multiphasic extraction systems discussed herein under FIGS. 3A and 3B.

Finally, the composition of the exemplary CO₂-EtOH-H₂O extraction system (226) is firstly controlled by a volumetric mixture of EtOH (230) and H₂O (232) to form a semi-aqueous mixture preferably ranging between 5%:90% EtOH:H₂O v:v and 30%:70% EtOH:H₂O v:v, and preferably at a temperature between −20° C. and 50° C., which incorporates a heated pressurized semi-aqueous extraction process followed by a much cooler CO₂ SALLE extract concentration and recovery process. As such, the preferred EtOH:H₂O mixture range has a δ_{T} between approximately 48 and 40 MPa^{1/2} at room temperature. The composition of the CO₂-EtOH-H₂O extraction system (226) is secondly controlled by the CO₂ pressure, preferably between 5 atm and 100 atm, to provide a volume of CO₂ gas or liquid (228) as a non-aqueous upper phase. Further to this, CO₂ (228) saturates the EtOH:H₂O semi-aqueous phase with aqueous CO₂, which expands and salts-out a portion of the dissolved EtOH (230) component to form a CO₂-expanded EtOH middle phase located between a lower semi-aqueous phase (principally H₂O) and upper non-aqueous phase (principally CO₂). The CO₂-expanded EtOH middle phase has a δ_{T} between approximately 20 and 26 MPa^{1/2}. The CO₂ (1) phase (228) selectively dissolves a portion of the EtOH (230) to form a CO₂ salted-out EtOH mixture, controlled by CO₂ pressure and semi-aqueous solution temperature, and provides a δ_{T} between approximately 17 and 20 MPa^{1/2}. Given this, the exemplary CO₂-EtOH-H₂O system (226) provides a δ_{T} ranging between approximately 17 MPa^{1/2} and 48 MPa^{1/2}, including a range of polarities and hydrogen bonding energies, to provide an optimal solvent environment for the many types of plant structures (200) and phytochemicals (212) found in a botanical system. Finally, process intensification techniques such as heating and ultrasonic homogenization may be used in the EtOH:H₂O semi-aqueous phase. Moreover, process intensification techniques such as a blade mixing or centrifugation may be used in the CO₂-EtOH-H₂O extraction system (226).

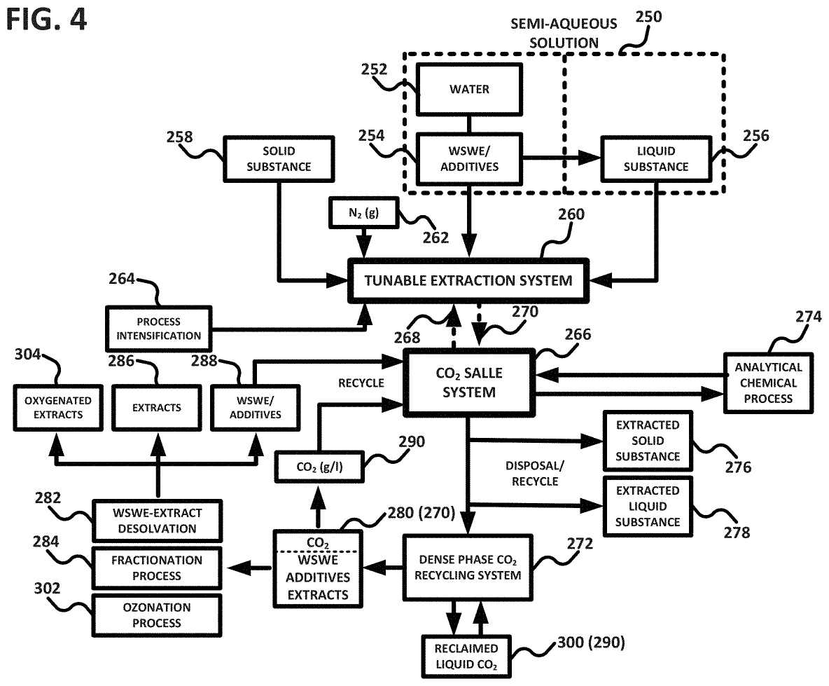

Having discussed the principal rationale for development of the present invention, following is a discussion, by reference to FIG. 4, of exemplary semi-aqueous liquid-liquid and solid-liquid extraction, concentration, and desolvation steps of the present invention used to extract virtually any type of liquid or solid substance to recover valuable organic compounds or environmental pollutants for instrumental analysis. FIG. 4 is a flowchart describing exemplary semi-aqueous liquid-liquid and solid-liquid extraction, concentration, and desolvation steps using the CO₂ SALLE processes of FIGS. 3A and 3B.

Now referring to FIG. 4, exemplary semi-aqueous solutions (250) used as primary extractants or as sources of expanded/salted-out co-solvents for dense phase CO₂ extraction/co-extraction processes comprise water (252) containing one or more water-soluble or water-emulsifiable (WSWE) compounds and optional additives (254). Bio-based, renewable, food safe, and/or low-toxicity compounds are preferred for use in the present invention as WSWE compounds and optional additives (254). Moreover, WSWE compounds (and blends containing same) are preferably formulated with a low freezing (melt) point, which is optimal for near-cryogenic desolvation and extract recovery methods used in the present invention. Different WSWE compounds, and blends of same, add different cohesion energies necessary to optimize chemical energy aspects of a semi-aqueous extraction solvent (i.e., matching solvent chemistry to plant structure and phytochemical solubility chemistries). WSWE compounds may comprise between 0.1% and 95% by volume of a WSWE:water composition. A WSWE:water composition may be used directly as a semi-aqueous extraction solvent, for example using WSWE compound concentrations between 0.1% to 30% by volume. Moreover, a WSWE:water composition may be formulated as a concentrate, for example using WSWE concentrations between 30% and 95% by volume, which is added to a second liquid to form a semi-aqueous extraction solvent. The resulting WSWE concentration is predetermined to be present in sufficient quantity and chemical composition to solvate a majority of predetermined or expected amounts of extract and to produce an adequate phase volume during CO₂ expansion and salting-out (phase separation) processes. Moreover, not all WSWE compounds are effectively expanded by dense phase CO₂, which is important for optimizing phase separation. As such, at least one CO₂-expandable WSWE component (i.e., ethanol) comprises a significant portion of a WSWE:water composition. WSWE compounds chosen from solvent groups comprising alcohols, ketones, and esters are exemplary CO₂-expandable solvent components of a particular WSWE:water composition. Finally, WSWE compositions are formulated to be at least partially miscible in dense phase CO₂ for effective co-extraction and formation of CO₂ salted-out solvent mixtures. Semi-aqueous solutions suitable for use as extractants or co-solvent adjuncts in the present invention may be formulated using one or more of the following exemplary WSWE compounds and optional additives (254):
1. Alcohols such as fermented ethanol (preferred), methanol, 1-propanol, 2-propanol, butanol, and oleyl alcohol;
2. Polyols (sugar alcohols) such as glycerol and erythritol;
3. Ketones such as acetone and butanone;
4. Esters such as ethyl acetate, ethyl lactate, propylene carbonate, oleic acid, glyceryl triacetate (triacetin), glyceryl diacetate (diacetin), and vegetable oils;
5. Nitriles such as acetonitrile;
6. Heterocyclic or poly ethers (collectively referred to as an “Ethers” herein) such as tetrahydrofuran and polyethylene glycol;
7. Organosulfur compounds such as dimethyl sulfoxide (DMSO) and diallyl disulfide (DADS);
8. Surfactants, cosurfactants, solubilizers, and emulsifiers (collectively referred to as a “Surfactants” herein) such as (preferably) plant-based or natural surfactants (cetearyl ethoxylate, cetyl ethoxylate, cetyl oleyl ethoxylate, lauryl ethoxylate, stearyl alcohol ethoxylate, castor oil ethoxylates and lauramine oxides); synthetic and natural surfactants such as sodium dodecyl sulfate (anionic surfactant), Triton X-100 (Polyoxyethylene octyl phenyl ether), PEG 2000 (polyethylene glycol), Brij-35 (polyoxyethylene), and soy lecithin (phospholipids); alcohols such as ethanol, 1-propanol, and butanol; and ozonated (oxygenated) extracts and natural compounds such as ozonated vegetable oils, ozonated terpenes, ozonated cannabinoids, ozonated flavonoids, and ozonated carotenoids;
9. Hydrotropes such as glyceryl triacetate (triacetin) and glyceryl diacetate (diacetin);
10. Emulsions, Microemulsions, and Nanoemulsions (collectively referred to as “Emulsions” herein) comprising mixtures of water, hydrocarbons, salts, surfactants, and cosurfactants;
11. Surfactant-free microemulsions (SFME) comprising oil, water, and a co-solvent or hydrotrope, which is miscible with water and oil;
12. CO₂-pressurized aqueous carbon dioxide solution (CO_{2 (aq)}) containing dissolved CO₂ gas, carbonic acid, bicarbonate anion, and carbonate anion; and
13. CO₂-expanded WSWE compounds.

Wherein, said one or more (preferably naturally derived) WSWE compounds are present in a semi-aqueous solution or a CO₂ salted-out solvent mixture at a concentration between 0.1% and 95% by volume. Further to this and in accordance with Hansen Solubility Parameters (Hansen 2007), the exemplary WSWE salting-out compounds, or blends of same, are chosen (or formulated) based on matching the dispersive (δ_{D}), polar (δ_{P}), and hydrogen-bonding parameters (δ_{H}) between the salting-out solvent(s) and the analyte(s) to be extracted from either the solid substance or liquid substance (performing as the aqueous solution), or both. This may also include computations for Solvent Interaction Radius (Ro) and Relative Energy Difference (RED). Still moreover, dense phase CO₂ (i.e., high pressure gas, saturated liquid phase, or supercritical state) serves as the relatively nonpolar salting-out agent and/or co-extractant in each liquid-liquid aqueous solvent scheme developed.

The volume of WSWE compound (i.e., organic solvent), and number of CO₂ SALLE cycles, needed for a particular application is determined using trial or bench tests which comprise HSP calculations, gravimetric measurements, or instrumental methods of analysis such as Gas Chromatography (GC), Raman Spectroscopy, and High-Performance Liquid Chromatography (HPLC). More preferably, CO₂ SALLE process development is performed in-situ and in real-time using light-induced fluorescence (LIF) spectroscopy.

An exemplary WSWE compound for use in the present invention is an emulsion. An emulsion is a dispersion of droplets of one liquid in a second immiscible liquid. The droplets are termed the dispersed phase, while the second liquid is the continuous phase. To stabilize an emulsion, a surfactant (i.e., lecithin) and cosurfactant (i.e., ethanol) are added such that the droplets remain dispersed and do not separate out as two phases. Depending on the phase, there are two types of microemulsions: water-in-oil (w/o) and oil-in-water (o/w). Water is the dispersed phase in w/o emulsions, whereas oil is the dispersed phase in o/w emulsions. One of the main differences between emulsions and microemulsions is that the size of the droplets of the dispersed phase of microemulsions is between 5 and 100 nm, while that of emulsions is >100 nm. Microemulsions are thermodynamically stable systems, whereas emulsions are kinetically stable systems. Still moreover, microemulsions are clear, thermodynamically stable isotropic liquid mixtures of hydrocarbons, water, and surfactant, frequently in combination with a cosurfactant, such as an alcohol. The aqueous phase may contain salt(s) and/or other ingredients. In contrast to ordinary emulsions, microemulsions form upon simple mixing of the components and do not require the high shear conditions generally used in the formation of ordinary emulsions.

Various surfactants, cosurfactants, and emulsifiers may be used to formulate emulsions and microemulsions. In this regard, natural nonionic or ionic plant-based surfactants, for example cetearyl ethoxylate and lecithin, are preferred for use in the present invention so that CO₂ salted-out organic mixtures containing these compounds may be formulated directly into tinctures or foods without toxicity concerns. For example, soy lecithin-ethanol-water mixtures may be used as green, low surface tension hydroethanolic emulsion extractants. During application of these surfactant-based semi-aqueous solutions in plant oil extraction applications, emulsions or microemulsions may form during processing.

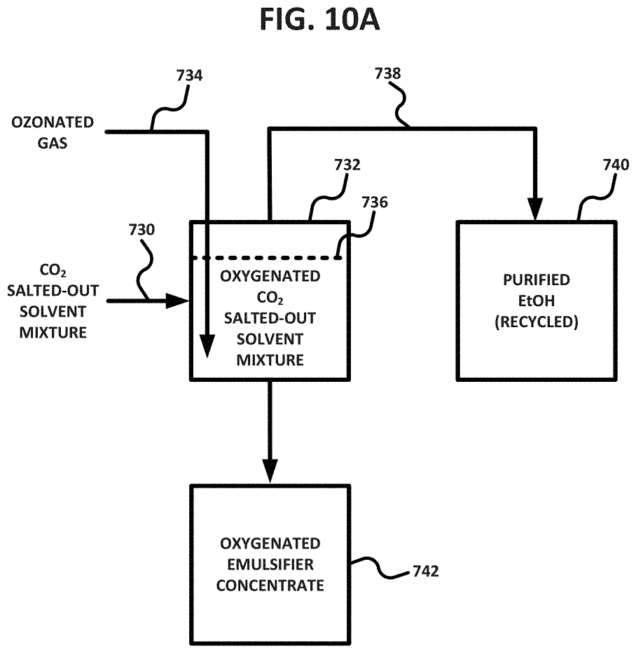

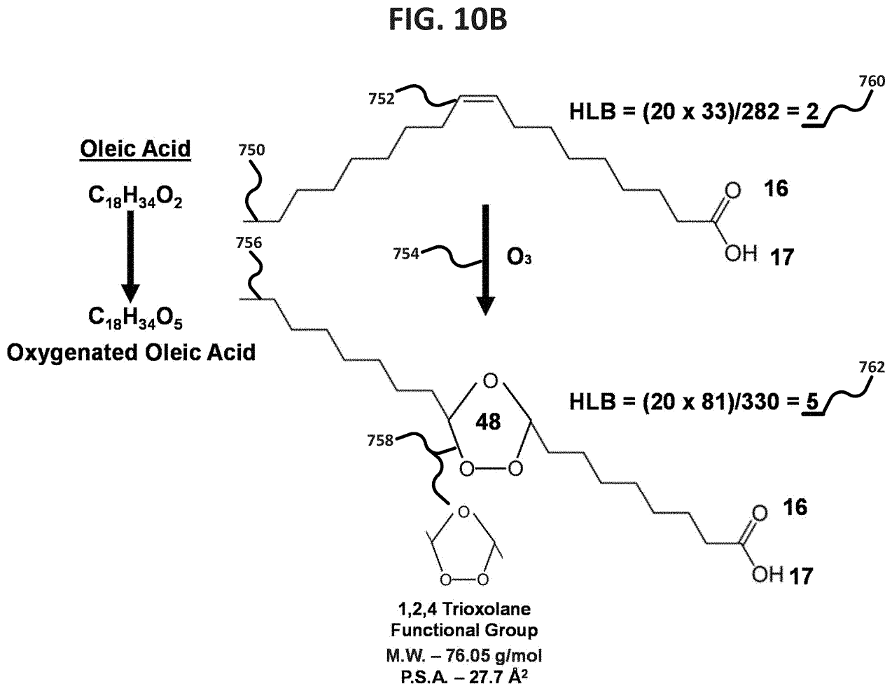

Moreover, a unique method for forming oxygenated emulsifying agents (and emulsions employing same) in-situ is disclosed herein under FIGS. 10A and 10B using selective ozonation of a portion of a natural unsaturated compound such as a vegetable oil, terpene, cannabinoid, flavonoid, or carotenoid. The ozonated natural extract is made more polar through the formation of one or more oxygenated functional groups (i.e., ozonide, trioxolane, epoxide, carboxylic acid, or peroxide), replacing one or more double bonds on the molecule. The oxygenation process is selective and quantitative based on time, temperature, degree of unsaturation, and ozone dose. Oxygenated compounds have higher hydrophilic-lipophilic balance (HLB) values and will spontaneously form an emulsion in aqueous solutions with water-insoluble compounds such vegetable oils.

For example, lycopene extract from tomato pomace is intended for use in the following food categories: baked goods, breakfast cereals, dairy products including frozen dairy desserts, dairy product analogues, spreads, bottled water, carbonated beverages, fruit and vegetable juices, soybean beverages, candy, soups, salad dressings, and other foods and beverages. Lycopene is a nonpolar compound that is insoluble in water, but can be selectively dissolved in various hydrocarbon solvents, oils, and blends of same.

A microemulsion used to extract lycopene from tomato pomace is described in Amiri-Rigi, A et al., “Extraction of Lycopene using a Lecithin-based Olive Oil Microemulsion, Food Chemistry 272 (2019) 568-573 (Amiri-Rigi et al). The microemulsion described in Amiri-Rigi et al. is composed of soy lecithin:1-propanol:olive oil:water (53.33:26.67:10:10 by wt. %). Tomato pomace (both skins and seeds) was chopped up using a blender and was added to centrifuge tubes containing the olive oil microemulsion, following which the centrifuge tubes were placed in a 35° C. shaking water bath for ° minutes to complete the extraction process. Subsequently, mixtures were centrifuged at 18,000 G-force for 15 minutes at room temperature and upper phase was decanted and its lycopene content was measured. The analysis revealed an 88% extraction efficiency. This biocompatible and food-grade microemulsion, following lycopene extraction, can be directly used in food formulations where it provides good solubility in aqueous and nonpolar media and improves the health-promoting properties of both lycopene and olive oil.

The work described under Amiri-Rigi et al. utilizes a concentrated microemulsion solution to obtain a concentrated mixture of microemulsion and relatively small amount of lycopene extract. This concentrated semi-aqueous extraction solution was employed at a ratio of 1 part tomato pomace to 5 parts extractant. Scaling this extraction process to higher production would require a tremendous amount of concentrated extractant, and a mechanical separation process such as a filter press or centrifuge to separate the biomass from the extractant. Moreover, the large amount of microemulsion extractant used to recover a very low concentration of lycopene extract from the tomato pomace (300 micrograms lycopene/g tomato pomace) may not be necessary.

As such, the present invention can replace the concentrated microemulsion extractant and high-G force centrifuge separation method of Amiri-Rigi et al. Dilute emulsion and microemulsion chemistries, as concentrated salted-out extractants, may be utilized to extract a large mass of wet tomato pomace. Process intensification techniques such as mixing, heating, centrifugation, and sonication may also be employed.

For example, a novel water-oxygenated olive oil-olive oil emulsion blend used in combination with dense phase CO₂ and process intensification techniques such as heating and ultrasonics can be used to extract lycopene from tomato peels. Moreover, a unique type of emulsifying agent of the present invention are ozonated (oxygenated) unsaturated organic compounds such as vegetable oils, terpenes, cannabinoids, flavonoids, and carotenoids.

In another example, lycopene is freely soluble in ethyl acetate (EA), a non-toxic water-soluble (86 g/L at 20° C.) and water-emulsifiable organic compound. As such, aqueous extraction solutions comprising water:EA:lecithin:olive oil or water:EA:lecithin:ethanol, for example, can be formulated and used as primary extractants for lycopene from tomato pomace using the present invention. Following each extraction cycle, the mixture is CO₂ expanded/salted-out to recover the dehydrated lycopene-lecithin-EA-oil mixture using a (HSP optimized) semi-aqueous-dense phase CO₂ extraction method of the present invention. Following this, the phase-separated water may be reformulated to form a dilute emulsion or microemulsion and reused. Moreover, aqueous solutions comprising water, surfactant, and ethyl lactate may be formulated for lycopene extraction as well.

Again, referring to FIG. 4, exemplary liquids or liquid substances (256), and mixtures of same, containing one or more extractable substances, and which also may be used alone as a naturally-containing WSWE semi-aqueous solution (i.e., alcoholic beverage) or as an ingredient in a semi-aqueous solution (i.e., water plus WSWE compound and optional additives) in a solid-liquid or liquid-liquid extraction process include:
1. Water such as purified waters—reverse osmosis purified water, dechlorinated water, activated carbon treated water, filtered water, distilled water, deionized water, or demineralized water; and potable waters—rainwater, reservoir water, lake water, stream water, tap water, or well water.
2. Alcoholic beverages such as one or a combination of beer, vodka, port, rum, gin, whiskey, bourbon, brandy, grain alcohol, cognac, tequila, wine, baijiu, sake, soju, hard seltzer water, and hard cider.
3. Fermentation broths containing valuable naturally fermented organic compounds.
4. Industrial wastewaters containing pollutants such as oils, metals, and toxic organic chemicals.
5. Fermented foods such as condiments and milks.
6. Water-based extractants derived from one or a combination of Soxhlet extraction, maceration, percolation, decoction, infusion, ultrasound-assisted extraction, pressurized liquid extraction, reflux extraction, subcritical water extraction, microwave-assisted extraction, enzyme-assisted extraction, hydro-distillation, or steam distillation processes. Moreover, liquid substances (256) may be a source of natural WSWE compounds (i.e., fermented beverages and foods) for use as co-solvents and flavor-infusing polyphenolic compounds in a dense phase CO₂ co-extraction process; or may be a source of valuable extracts (i.e., fermentation broths containing CBD); or may contain environmental pollutants requiring instrumental analysis; or may be extractants derived from water-based extraction processes (i.e., subcritical water extraction).

Finally, exemplary liquid substances (256) used for practicing the present invention may contain a significant amount of water with only minimal amounts of natural WSWE compounds, termed dilute liquid substances or solutions. Dilute liquid substances may be re-formulated as more concentrated semi-aqueous solutions by introducing additional WSWE compounds (254). Still moreover, exemplary liquid substances (256) may be mixed with optional WSWE additives such as, for example, water, organic acids and salts, inorganic salts (i.e., Sea Salt, NaCl, K₂CO₃, Na₂SO₄, K₃PO₄, etc.), natural or non-toxic surfactants and cosurfactants, enzymes, pH buffers, chelation agents, and ozone, among other additives which enhance extraction, recovery, or analytical processes herein.

Further to this, exemplary liquid substances may contain naturally fermented water-soluble organic solvents, and organic solvent-soluble compounds, for example fermented ethanol (EtOH) and EtOH-soluble fermented organic compounds, or may be mixed with semi-aqueous solutions (250) containing WSWE compounds and additives (254) such as alcohols, ketones, esters, vegetable oils, nitriles, inorganic salts, and organic acids and salts, among many other examples, and prior to liquid-liquid or solid-liquid extraction processes and CO₂ SALLE processes described herein.

For example, with regards to alcoholic beverages, low alcohol content beverages such as beers and wines may be blended with high alcoholic content beverages such as a higher-proof grain alcohol to boost natural fermented ethanol content levels of the mixture while retaining natural flavonoids present in the beers and wines. Blending is useful for producing a minimum volume of infused ethanol extract for effective dense phase CO₂ solid-liquid co-extraction and for formulating natural tinctures, vapes, or for use as food and beverage additives.

Most legal sources and supplies of grain-based or bio-based ethanol, also termed bio-EtOH herein, for botanical material extraction is also called “denatured ethanol”. Denatured ethanol typically contains up to 10% denaturant compounds (by vol.) that make it poisonous, bad-tasting, foul-smelling, nauseating, or otherwise non-drinkable. Exemplary denaturants include methanol, isopropyl alcohol, acetone, methyl ethyl ketone, and heptane. Adding these denaturants discourages recreational consumption.

The reasons for this are straightforward. Sales of alcoholic beverages are heavily taxed for both revenue and public health policy purposes. To avoid paying beverage taxes on alcohol that is not meant to be consumed, the alcohol must be denatured, or treated with added chemicals to make it unpalatable. Denatured alcohol is used identically to ethanol itself except for applications that involve fuel, surgical and laboratory stock. Pure ethanol is required for food and beverage applications and certain chemical reactions where the denaturant would interfere. As denatured ethanol is sold without the often-heavy taxes on alcohol suitable for consumption, it can be a much lower cost and purely organic solution for most uses that do not involve drinking, for example botanical material extraction.

Although denaturing ethanol does not chemically alter the ethanol molecule and its performance in a botanical extraction process, it is intentionally difficult to separate the denaturing component using conventional separation methods such as distillation or membrane filtration processes. However, the downside is that these same denaturants (i.e., poisons) end up as trace components within botanical extraction products such as tinctures and oils. As already discussed herein, it is becoming more desirable to produce completely natural and non-toxic botanical extracts and compounds using organically grown botanical materials absent of pesticides and heavy metals, as well as pure unadulterated extraction solvents.

Given this, a 100% organic solution to this constraint is to utilize already taxed and unadulterated commercial alcoholic beverages. One exemplary source is a commercial product called Everclear Grain Alcohol, 190 Proof, available from select alcoholic beverage supply stores (and U.S. States). The 190-proof variation of Everclear is 92.4% ethanol by weight, which is produced at approximately the practical limit of distillation purity (95% EtOH:5% Water). However, many U.S. States impose limits on maximum alcohol content or have other restrictions that prohibit the sale of the 190-proof variation of Everclear, and several of those States also effectively prohibit lower-proof Everclear grain alcohol.

Still moreover, the problem with low-proof grain alcohol is that it is ineffective as a solvent for most botanical material extraction applications, particularly for botanical materials containing target extractable compounds which do not exhibit appreciable water solubility under S.T.P. conditions.

The present invention provides novel methods and processes for effectively utilizing commercial alcoholic beverages as liquid substances (256) in liquid-liquid and solid-liquid extraction processes of the present invention. Suitable alcoholic beverages range from dilute aqueous alcohol solutions (i.e., Beers, Wines, Ports, etc.) to concentrated aqueous alcoholic solutions (i.e., Whiskeys, Vodkas, Grain Alcohols, etc.), and include blends of same and with various custom additives.

Commercially available alcoholic beverages are excellent sources of naturally fermented ethanol and a wide variety of naturally fermented EtOH-soluble and dense phase CO₂-soluble organic compounds. During a solid-liquid extraction process, naturally fermented WSWE compounds may be co-extracted and incorporated into a biomaterial extract to impart healthful characteristics or pleasant flavors, colors, and aromas, to form an infused biomaterial extract or tincture.

TABLE 4
Exemplary Alcoholic Beverages and Chemistries

| | Maximum | |
|---|---|---|
| Alcoholic | EtOH Content | EtOH-Soluble Compounds |
| Beverage | (% by Vol.)^{/1} | (Exemplary Fermentation-Distillation By-Products and Additives) |
| Beer | 14% | Alpha Acids, Beta Acids, Essential Oils, Esters |
| Vodka | 95% | Ethanol Hydrates, Citric Acid, Organic Alcohols, Glycerol, Coumarin |
| Port | 20% | Anthocyanins, Sotolon, Whisky Lactones |
| Rum | 75% | Esters, Vanillin, Gualacol, Organic Acids, Organic Alcohols, |
| Gin | 68% | Juniper Berry Compounds, Limonene, Myrcene, Linalool, Geranyl Acetate |
| Whiskey | 65% | Whiskey Lactones, Aldehydes, Esters, Phenolics, Organic Alcohols |
| Tequila | 40% | Isovaleraldehyde, Isoamyl Alcohol, B-Damascenone, Vanillin |
| Red Wine | 12% | Anthocyanins, Tannins, Flavan-3-ols, Flavonols |
| Baijiu | 65% | Organic acids, Esters, Lactones, Phenols, Heterocycles, Terpenes, Aromatics |

### 1. Very High EtOH Content Alcoholic Beverages May not be Commercially Available.

Exemplary alcoholic beverages and chemistries are shown in Table 4. There are many types and sources of both fermented and distilled alcoholic beverages, and innumerable blends and additives, suitable as liquid substances (256) for practicing liquid-liquid and solid-liquid extraction and extract recovery methods and processes of the present invention.

Some of which are exotic, ancient, and contain very healthful (ethanol and CO₂ solvent-soluble) ingredients, such as Baijiu, an ancient Chinese liquor and is the national liquor of China. The production of baijiu is different from that of other exemplary distilled liquors listed in Table 4 because it combines the two distinctive processes of fermentation and distillation. It may also be unique from a human health perspective as well. According to Liu, H. and Sun, B., “Effect of Fermentation Processing on the Flavor of Baijiu”, J. Agric. Food Chem., 2018, 66, pp. 5425-5432 (Liu and Sun), Liu and Sun state that Baijiu is rich in many flavor components, including organic acids (such as acetic, citric, lactic, malic, tartaric, and linoleic acids) and salts, esters (such as ethyl acetate, ethyl lactate, and ethyl hexanoate), lactones, phenols, heterocycles, terpenes, and aromatic compounds. Furthermore, Baijiu contains potential functional components, such as amino acids and peptides which are beneficial to humans. The first economic history book from China, “Shi-Huo-Zhi” by Ban Gu, reported that Baijiu has long been used as a base for traditional Chinese medicine, at least since the Eastern Han dynasty.

Exemplary liquid substances (256) may be mixed with semi-aqueous solutions containing water-soluble additives such as, for example, organic acids and salts, inorganic salts (i.e., Sea Salt, NaCl, K₂CO₃, Na₂SO₄, K₃PO₄, etc.), natural or non-toxic surfactants and cosurfactants, enzymes, pH buffers, chelation agents, and ozone, among other additives.

In FIG. 4, solid substances (258) containing one or more extractable substances, and used in combination with a semi-aqueous solution (250) and/or a liquid substance (256), as well as dense phase CO₂ co-extraction and extract concentration and recovery processes, are used in solid-liquid extraction processes of the present invention. Many different types of solid substances (258) can be extracted (or co-extracted) using the various processes of the present invention. Solid substances (258) may be dry or wet (fresh or crude). For example, solid substances (258) may contain extractable compounds which are flavorful or healthful; or may contain proteins or other organic compounds of interest (i.e., animal tissues); or may contain environmental contaminants which require an analytical chemical process (i.e., contaminated soils) to identify and quantify same.

Exemplary solid substances (258), and mixtures of same, include:
1. Natural products such as nuts, spices, herbs, hops, roots, dried fruits, bark, hemp, and psychoactive plants such as Cannabis sativa, cannabis indica, and Cannabis ruderalis.
2. Pomaces (food wastes skins, stems, seeds) such as tomato pomace, carrot pomace, apple pomace, and grape pomace.
3. Animal tissues such as skin, hair, bones, muscles, and organs.
4. Soils such as ocean outfall sediments, USEPA superfund site soils.
5. Semi-solids such as sludge and slurry.

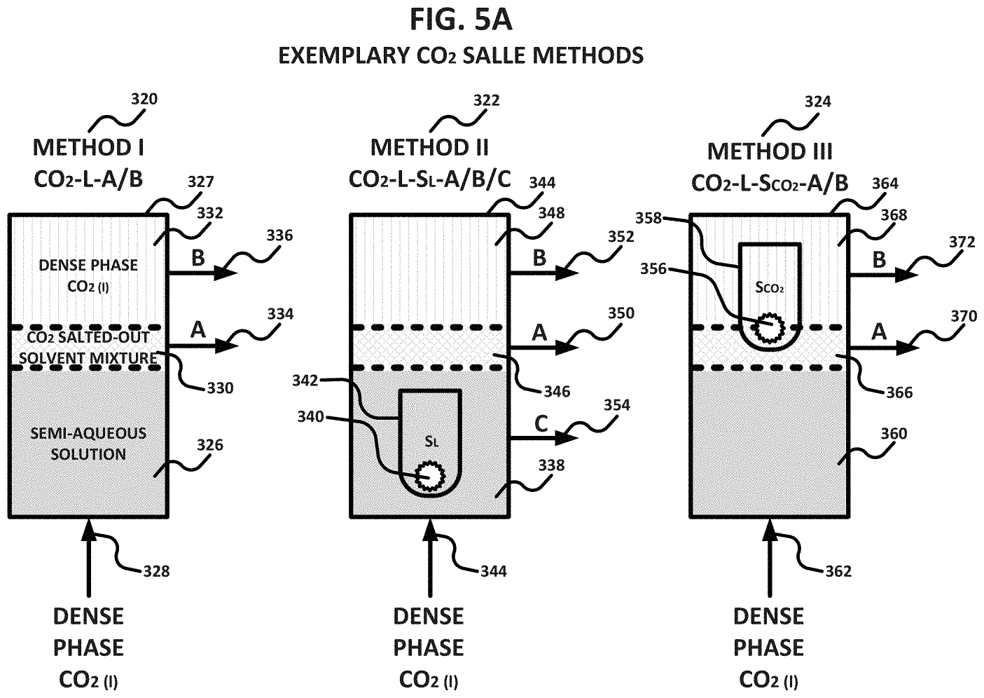

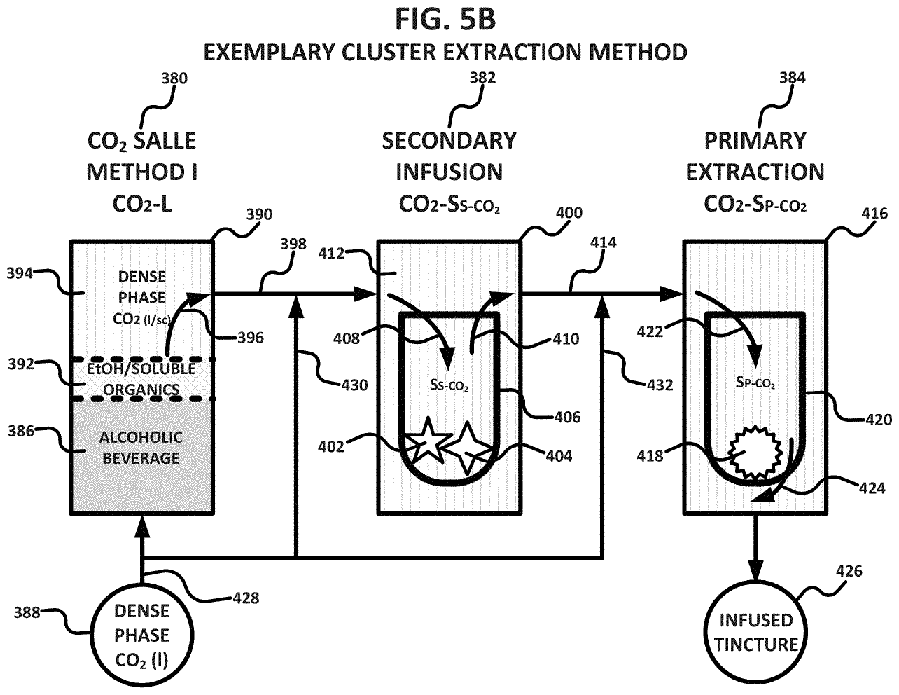

Further to this, and discussed under FIG. 5B herein, one or more solid substances (258) may be combined as a primary and secondary solid substance mixture. The secondary solid substance, containing natural solvent modifiers, flavor enhancers, or healthful additives, is co-extracted with the primary solid substance, containing the desired or target extract (i.e., CBD). This co-extraction process is referred to as a “cluster extraction” herein.

Still referring to FIG. 4, an exemplary semi-aqueous solution (250), liquid substance (256), and solid substance (258) are used in a tunable extraction system (260) in various combinations and proportions, as follows:
1. Semi-Aqueous Solution (250) and Liquid Substance (256);
2. Semi-Aqueous Solution (260) and Solid Substance (258);
3. Liquid Substance (256), WSWE and Additives (254), and Solid Substance (258); and
4. Semi-Aqueous Solution (250), Liquid Substance (256), and Solid Substance (258).

Moreover, said one or more solid substances (258) may be co-extracted together in said semi-aqueous solution, or in a dense phase CO₂ or CO₂ salted-out solvent mixture during a subsequent CO₂ SALLE process. Alternatively, said one or more solid substances (258) may be co-extracted separately in said semi-aqueous solution, or in a dense phase CO₂ or CO₂ salted-out solvent mixture, for example as described in a cluster extraction process under FIG. 5B.

An exemplary extraction process used in a tunable extraction system in combination with a CO₂ SALLE process is a modified subcritical water extraction (MSWE). Again, referring to FIG. 4, a semi-aqueous solution (260) is preheated to, for example, 100° C., and introduced into a tunable extraction system (260) containing a solid substance (256). If the target extract contained in the solid substance (256) is subject to oxidation or is more efficiently extracted under near-neutral pH conditions, nitrogen gas (N_{2 (g)}) (262) is used to purge oxygen and pressurize said semi-aqueous solution (260). A N_{2 (g)} (262) atmosphere provides a vapor pressure blanket that reduces oxidative reactions and prevents the semi-aqueous solution (260) from boiling at solution extraction temperatures exceeding 100° C. Moreover, and if used, it is preferred to apply process intensification techniques (264) such as ultrasonic agitation during a solid-liquid extraction process such as subcritical water extraction. Other process intensification processes such as mixing and centrifugation may also be used. Following the MSWE-solid extraction process, for example performed at 125° C. and a N_{2 (g)} atmospheric pressure of 5 atm, a water-based extractant is produced which contains one or more dissolved extracts. The water-based extractant is transferred to a CO₂ SALLE system (266), and preferably cooled to below 30° C. during transfer, and further processed using an exemplary CO₂ SALLE process, for example as discussed under FIGS. 3A and 3B. In an exemplary CO₂ SALLE process, liquid CO₂ (268) is injected into the water-based extractant, mixed into, and stratified to produce a CO₂ salted-out solvent mixture (270) phase containing both solvated and desolvated extracts removed from said water-based extractant. Said CO₂ salted-out solvent mixture is withdrawn from the CO₂ SALLE system (266) and transferred to a dense phase CO₂ recycling system (272). The exemplary CO₂ SALLE process thus described may be repeated, including the addition of extra WSWE compounds, as required to completely remove solvated and desolvated extracts from the water-based extractant. Moreover, this process may be monitored and controlled in-situ by an analytical chemical process (274), for example, using ultraviolet-visible (UV-VIS) spectroscopy, light-induced fluorescence spectroscopy, or high-performance liquid chromatography (HPLC), as well as analytical chemical methods such as gravimetric analysis and density measurements (semi-aqueous solution).

Still referring to FIG. 4, upon completion of said CO₂ SALLE process, and transfer of said CO₂ salted-out solvent mixtures (270) to said dense phase CO₂ recycling system (272), extracted solid substance (274) and extracted liquid substance (water-based extractant) (278) are removed from the tunable extraction system (260). Following this, the CO₂ salted-out solvent mixture (270) comprising liquid CO₂-WSWE-Additives-Extracts (280) is separated into component parts using processes including a desolvation process (282) such isostatic pressure distillation or near-cryogenic capillary crystallization, or a fractionation process (284) such as thin-film vacuum distillation. The products of these separation processes include isolated or fractionated extracts (286), WSWE compound and additives (288), and CO₂ gas (290), which may be compressed, cooled, and stored as reclaimed liquid CO₂ (300). As shown in FIG. 4, the separated WSWE-Additives (288) and CO₂ (290) may be recycled and reused in said CO₂ SALLE system (266). Finally, the recovered CO₂ salted-out solvent mixture (270), with the CO₂ gas component removed, may be treated using an ozonation process (302) to produce oxygenated extracts (304), discussed under FIGS. 10a and 10B herein.

Having discussed exemplary aspects of a tunable extraction system used in combination with a CO₂ SALLE process, following is a description of three exemplary CO₂ SALLE methods derived from FIGS. 3A, 3B, and 4 for use in a liquid-liquid and solid-liquid extraction process. Now referring to FIG. 5A, exemplary liquid and solid substances discussed under FIG. 4 may be processed using exemplary CO₂ SALLE Methods I (320), II (322), and III (324), and each method is followed by one or more exemplary Desolvation and Extract Recovery Methods A, B, and C, described as follows:

CO₂ SALLE Method I: CO₂-L-A/B (320)

In this exemplary CO₂ SALLE method, a semi-aqueous solution (326) containing one or more WSWE compounds and optional additives (naturally present or purposely added) and containing one or more dissolved target extracts, is expanded/salted-out and co-extracted using dense phase CO₂ (328) to recover said extracts from said semi-aqueous solution (326), the method comprising:
1. A semi-aqueous solution (326) comprising 100 Proof Vodka (approximately 50% by volume fermented ethanol (i.e., a natural WSWE compound) and 50% by volume water), is placed in a pressure vessel (327);
2. Dense phase CO₂ (328) is injected and bubbled (as a near-cryogenic CO₂ gas-solid aerosol) through said semi-aqueous solution (326) to cool and saturate with CO₂ to a pre-determined temperature between about 20° C. and −40° C. at a sublimating vapor pressure of about 3 atm (i.e., continuously venting to atmosphere), following which said cooled and CO₂-saturated semi-aqueous solution is autogenously pressurized (vis-à-vis sublimation pressurization and temperature rise) or mechanically pressurized with dense phase CO₂ (328) using a pump (preferred) to a pressure between 5 atm and 90 atm to selectively (and volumetrically) expand/salt-out to form a fermented ethanol-rich CO₂ salted-out solvent mixture (330) above said semi-aqueous solution and a liquid CO₂-rich CO₂ salted-out solvent mixture (332) above said ethanol-rich phase (330). The multiphasic mixture thus formed is preferably turbulently mixed and allowed to stratify into distinct phases as shown;
3. A portion of said fermented ethanol-rich CO₂ salted-out solvent mixture (330) is subsequently dissolved into said liquid CO₂-rich phase (332); and
4. Said CO₂ salted-out solvent mixtures are desolvated to recover fermented ethanol and CO₂.

Exemplary Desolvation and Extract Recovery Methods A and B comprise the following:

Desolvation-Extract Recovery Method A (334): CO₂ salted-out solvent mixture phase (330), which is rich in fermented EtOH, is decanted under CO₂ gas pressure for extract concentration and recovery, for example, using isostatic pressure distillation. Alternatively, the CO₂ salted-out solvent mixture (330) is decanted and analyzed using an analytical chemical process, for example, using instrumental techniques such as high-performance liquid chromatography (HPLC), gas chromatography (GC), UV-VIS spectroscopy, and light-induced fluorescence (LIF) spectroscopy.

Desolvation-Extract Recovery Method B (336): CO₂ salted-out solvent mixture phase (332), which is rich in liquid CO₂, is decanted under CO₂ gas pressure for extract concentration and recovery, for example, using a near-cryogenic CO₂ gas-solid aerosol spray separation process. Alternatively, the CO₂ salted-out solvent mixture (332) is decanted and analyzed using an analytical chemical process, for example, using instrumental techniques such as high-performance liquid chromatography (HPLC), gas chromatography (GC), UV-VIS spectroscopy, and light-induced fluorescence (LIF) spectroscopy.

CO₂ SALLE Method II: CO₂-L-S_{L}-A/B/C (322)

In this exemplary CO₂ SALLE method, a semi-aqueous solution (338) containing a WSWE compound (naturally present or purposely added) is co-extracted with a liquid-immersed solid substance (S_{L}) (340) containing one or more soluble extracts, and which is contained in a porous container or centrifuge basket (342). The semi-aqueous solution (338) and solid substance (S_{L}) (340) mixture are expanded/salted-out and co-extracted with dense phase CO₂ (344) to extract and recover soluble extracts, the method comprising:
1. A solid substance (S_{L}) (340) containing one or more dissolved target extracts, and enclosed within a porous container or centrifuge basket (342), is positioned within a pressure vessel (344);
2. Said pressure vessel (344) containing said solid substance (S_{L}) (342) is filled with a semi-aqueous solution (338) containing an extraction mixture comprising 60% by vol. water, 40% by vol. ethyl acetate, and in contact with immersed solid substance (S_{L}) (342);
3. Dense phase CO₂ (344) is injected and bubbled (as a near-cryogenic gas-solid aerosol) through said semi-aqueous solution to cool and saturate the semi-aqueous solution (338) and solid substance (S_{L}) (340) with CO₂ to a pre-determined temperature between about 20° C. and −40° C. at a sublimating vapor pressure of about 3 atm (i.e., pressure maintained by continuously venting sublimated CO₂ gas to atmosphere), following which said cooled and CO₂-saturated semi-aqueous solution (338) and immersed solid substance (S_{L}) (342) is autogenously pressurized (vis-à-vis sublimation pressurization and temperature rise with the pressure vessel (344) vent closed) or mechanically pressurized with dense phase CO₂ using a pump (preferred) to a pressure between 5 atm and 90 atm to selectively (and volumetrically) salt-out and form an ethyl acetate-rich CO₂ salted-out solvent mixture (346) above said semi-aqueous solution (338) and a liquid CO₂-rich CO₂ salted-out solvent mixture (348) above said ethyl acetate-rich phase (346). The multiphasic mixture thus formed is preferably turbulently mixed and allowed to stratify into distinct phases as shown;
4. A portion of said salted-out ethyl acetate-rich CO₂ salted-out solvent mixture (346), containing one or more dissolved extracts removed from the solid substance (S_{L}) (340), is subsequently dissolved into said CO₂-rich CO₂ salted-out solvent mixture (348); and
5. Said CO₂ salted-out solvent mixtures (346, 348) are desolvated to recover dissolved extracts, ethyl acetate, and CO₂. Moreover, said semi-aqueous solution (338) is decanted for additional processing and recycled back into the exemplary CO₂ SALLE method.

Exemplary Desolvation and Extract Recovery Methods A, B, and C comprise the following:

Desolvation-Extract Recovery Method A (350): CO₂ salted-out solvent mixture phase (346), which is rich in ethyl acetate, is decanted under CO₂ gas pressure for extract concentration and recovery, for example, using isostatic pressure distillation. Alternatively, the CO₂ salted-out solvent mixture (346) is decanted and analyzed using an analytical chemical process, for example, using instrumental techniques such as high-performance liquid chromatography (HPLC), gas chromatography (GC), UV-VIS spectroscopy, and light-induced fluorescence (LIF) spectroscopy.

Desolvation-Extract Recovery Method B (352): CO₂ salted-out solvent mixture phase (348), which is rich in liquid CO₂, is decanted under CO₂ gas pressure for extract concentration and recovery using, for example, distillation or near-cryogenic CO₂ solid-gas aerosol spray desolvation. Alternatively, the CO₂ salted-out solvent mixture (348) is decanted and analyzed using an analytical chemical process, for example, using instrumental techniques such as high-performance liquid chromatography (HPLC), gas chromatography (GC), UV-VIS spectroscopy, and light-induced fluorescence (LIF) spectroscopy.

Desolvation-Extract Recovery Method C (354): CO₂ salted-out solvent mixtures (346, 348) (including solubilized and suspended compounds) and semi-aqueous solution (338) are decanted under CO₂ gas pressure for further processing such as centrifugation, Dissolved CO₂ Flotation (DCF), and oil skimming to recover extracted and precipitated extracts from the surface of the CO₂ salted-out aqueous solution. Once processed to remove extracted compounds, said processed CO₂ salted-out semi-aqueous solution may be recycled back into the original extraction process for reuse.

CO₂ SALLE Method III: CO₂-L-S_{CO2}-A/B (324):

In this exemplary CO₂ SALLE method, a solid substance (S_{CO}_{₂}) (356) containing one or more soluble extracts, and which is contained in a porous container or centrifuge basket (358), is positioned above a semi-aqueous solution (360) containing a WSWE compound (naturally present or purposely added). The extraction system comprising said semi-aqueous solution (360) and solid substance (S_{CO}_{₂}) (356) is expanded/salted-out and co-extracted using dense phase CO₂ (362) to recover extracts from said solid substance (S_{CO}_{₂}) (356), the method comprising:

1. A pressure vessel (364) is partially filled with a semi-aqueous solution (360), which comprises water and a water-soluble water-emulsifiable compound (and optionally other additives);

2. A solid substance (S_{CO2}) (356) containing one or more extracts, and contained within a porous container or centrifuge basket (358), is positioned above said semi-aqueous solution (360) within said pressure vessel (364);

3. Dense phase CO₂ (362) is injected and bubbled (as a near-cryogenic CO₂ gas-solid aerosol) through said semi-aqueous solution (360) to cool and saturate the extraction system comprising semi-aqueous solution (360) and solid substance (S_{CO2}) (356) with CO₂ to a pre-determined temperature between about 20° C. and −40° C. at a sublimating vapor pressure of about 3 atm (i.e., pressure maintained by continuously venting to atmosphere), following which said cooled and CO₂-saturated semi-aqueous solution (360) and solid substance (S_{CO2}) (356) is autogenously pressurized (vis-à-vis sublimation pressurization and temperature rise with the pressure vessel (364) vent valve closed) or mechanically pressurized with dense phase CO₂ (362) using a pump (preferred) to a pressure between 5 atm and 90 atm to selectively (and volumetrically) salt-out and form a WSWE-rich CO₂ salted-out solvent mixture (366) as a phase containing said extracts above said semi-aqueous solution (360) and a liquid CO₂-rich CO₂ salted-out solvent mixture (368) above said WSWE-rich phase. The multiphasic mixture thus formed is preferably turbulently mixed and allowed to stratify into distinct phases as shown;

4. A portion of said WSWE-rich CO₂ salted-out solvent mixture (366) containing one or more dissolved extracts removed from said solid substance (S_{CO2}) (356) is subsequently dissolved into said liquid CO₂-rich CO₂ salted-out WSWE mixture (368); and

5. Said WSWE-rich and Liquid CO₂-rich CO₂ salted-out solvent mixtures (366, 368) are desolvated to recover solvated and desolvated extracts, WSWE, and CO₂.

Exemplary Desolvation and Extract Recovery Methods A and B comprise the following:

Desolvation-Extract Recovery Method A (370): WSWE-rich CO₂ salted-out solvent mixture (366) phase is decanted under CO₂ gas pressure for extract concentration and recovery, for example, using isostatic pressure distillation. Alternatively, WSWE-rich CO₂ salted-out solvent mixture (366) is decanted and analyzed using an analytical chemical process, for example, using instrumental techniques such as high-performance liquid chromatography (HPLC), gas chromatography (GC), UV-VIS spectroscopy, and light-induced fluorescence (LIF) spectroscopy.

Desolvation-Extract Recovery Method B (372): Liquid CO₂-rich CO₂ salted-out solvent mixture (368) is decanted under CO₂ gas pressure for extract concentration and recovery using, for example, distillation or near-cryogenic CO₂ solid-gas aerosol spray desolvation. Alternatively, Liquid CO₂-rich CO₂ salted-out solvent mixture (368) is decanted and analyzed using an analytical chemical process, for example, using instrumental techniques such as high-performance liquid chromatography (HPLC), gas chromatography (GC), UV-VIS spectroscopy, and light-induced fluorescence (LIF) spectroscopy.

Moreover, exemplary CO₂ SALLE Methods I, II, and III may be operated at subcritical water-supercritical CO₂ solvent system temperatures as high as 300° C. and CO₂ pressures as high as 5,000 psi (340 atm), in a CO₂-solvent modified subcritical water solid-liquid extraction process. However, it is preferred that the semi-aqueous extractant temperature be reduced (cooled) to below 100° C., and most preferably below 30° C., prior to phase stratification and desolvation steps to maximize CO₂ expanded/salted-out WSWE phase volume and to prevent boiling and formation of high temperature water vapor.

Still moreover, the prior art establishes that efficient subcritical water extractions are possible at temperatures of 150° C. or lower and vapor pressures of 20 atm or lower in many different solid-liquid extraction applications. This is an important aspect because higher processing temperatures waste energy and decompose or denature labile organic extracts. In this regard, and discussed in detail under FIG. 6A, replacing neat water with dilute hydroethanolic solutions useful for practicing the present invention further reduce subcritical water extraction temperatures. For example, an 80%:20% by vol. H₂O:EtOH subcritical water extractant operating at 225° C. and 25 atm produces an alcohol-like extraction solvent chemistry similar to 100% water (by vol.) operating at 300° C. and 85 atm, while providing a convenient salting-out WSWE compound mixture for CO₂ SALLE processing in accordance with the present invention.

Still moreover, liquid and/or solid substances may be pretreated before or during CO₂ SALLE Method I, II, and III using process intensification techniques such as grinding, ultrasonics (US), microwaves (MW), and centrifugation (CF) to enhance extraction, desolvation, and extract recovery processes of the present invention. Treatment techniques (pre-treatments and in-situ treatments) employing high frequency (i.e., 20/40 kHz) or low frequency (i.e., 300 Hz) acoustics, 2.45 GHz microwaves, and bi-directional centrifugation are used herein to intensify the CO₂ SALLE process to improve extraction efficiency and the recovery of valuable compounds.

FIG. 5B is a schematic describing a novel CO₂ SALLE co-extraction process called a cluster extraction. A cluster extraction combines CO₂ SALLE Method I of FIG. 5A with one or more conventional dense phase CO₂ extraction processes to produce an infused botanical extract. A cluster extraction involves the sequential or simultaneous co-extraction of solid and liquid substances (all containing different and synergistic organic compounds. For example, co-extracted organic compounds can enhance dense phase CO₂ (liquid or supercritical) extraction efficiency vis-à-vis co-solvency effects. In another example, co-extracted organic compounds change or improve the quality (i.e., healthfulness, aroma, color, or flavor) of the final extract.

An exemplary cluster extraction application is described under FIG. 5B. During CO₂ expansion/salting-out and extraction of organic components from a fermented aqueous alcoholic solution into a dense phase CO₂, for example liquid CO₂, an alcoholic beverage extract-infused CO₂ salted-out solvent mixture is formed and used as a solid substance extractant. Said alcoholic beverage-infused extractant may be used directly in a primary botanical extraction. Alternatively, said alcoholic beverage-infused extractant may be further modified by using same to extract organic compounds contained in one or more secondary botanical solid substances to form an alcoholic beverage-infused and botanical solid extract-infused extractant. There are many commercially available fermented liquids and botanical solids which serve as a huge source of natural, non-toxic, and mixed organic compounds. Besides improving botanical compound extraction performance, fermented liquid and botanical solid organic compounds incorporate healthy and flavorful chemistry into the primary biomaterial extract. For example, fermented ethanol and ethanol-soluble organic additives such as humulene are simultaneously dissolved into a liquid CO₂ solvent which assist with the extraction of organic compounds from one or more solid biomaterials. Said modified liquid phase CO₂ is used to extract flavorful extracts from a secondary solid substance such mint or lavender to form an extract-infused liquid CO₂ extraction solvent mixture for a primary solid substance such as cannabis, to form a mint or lavender flavor-infused CBD/THC tincture.

In this regard, fermented and botanical solid organic compounds dissolved in a dense phase CO₂-ethanol mixture behave as co-extractants. These co-extractants beneficially modify the solubility chemistry of the dense phase CO₂, imparting a broader spectrum of functional group chemistries and associated dispersive, polar, and hydrogen bonding properties. Prior art research establishes that a mixture of secondary natural co-extractants used with a primary extraction solvent and biomaterial improves the dynamics and performance of the extraction process through synergistic changes in the overall solubility chemistry and transport phenomenon associated with the biomaterial solid-liquid solvent extraction system.

An example of the co-extraction effect is found in Ciurlia, L. et al., “Supercritical Carbon Dioxide Co-Extraction of Tomatoes (Lycopersicum esculentum L.) and Hazelnuts (Corylus avellana L.): A New Procedure in Obtaining a Source of Natural Lycopene”, J. of Supercritical Fluids, 49 (2009) 338-344, (Ciurlia et al.). Ciurlia et al. performed a supercritical CO₂ extraction test comprising dried tomato powder mixed with ground roasted hazelnuts to simultaneously co-extract lycopene from the tomatoes and oils (and other compounds) from the hazelnuts. This extraction procedure was compared to a separate supercritical CO₂ extraction procedure under the same pressure, temperature, and flow conditions using liquid hazelnut oil mixed with tomato powder. Ground hazelnut solid co-extraction resulted in greater than 70% lycopene recovery, while hazelnut oil as a co-solvent in scCO₂ resulted in only 30% recovery. In the co-solvent process, the oil extraction was rapid at the beginning of the process, as the oil was transported and not extracted by the supercritical fluid. On the contrary, the co-extraction process showed that the hazelnut oil was gradually extracted from solid hazelnuts with a trend representing a two-mechanism extraction process. Ciurlia et al. hypothesized that a diffusion-controlled extraction of embedded oil in the ground hazelnuts allows a better solubilization of lycopene (over time) into co-extracted hazelnut oil. This diffusion-controlled mechanism enables more efficient lycopene extraction, with the consequent increase of lycopene yield as compared to CO₂ dopants or co-solvents.

Another example of the co-extraction effect is found in Aris, et al., “Effect of Particle Size and Co-Extractant on Momordica Charantia Extract Yield and Diffusion Coefficient using Supercritical CO₂”, Malaysian Journal of Fundamental and Applied Sciences, Vol. 14, No. 3, (2018), 368-373 (Aris et al.). Aris et al. determined that co-extracting biomaterial Momordica Charantia pre-soaked in methanol with supercritical CO₂ increased extraction efficiency of the target compound, charantin. In addition, pre-grinding the Momordica Charatia to a particle size of 0.3 mm was found to be optimal for extraction efficiency. Aris et al. concluded that mean particle size of 0.3 mm gave the highest extract yield of 3.32% and 1.34% respectively for with and without the methanol co-extractant, respectively. Moreover, the value of the diffusion coefficient (Dₑ) at 0.3 mm mean particle size, with and without the methanol co-extractant was determined to be 8.820×10⁻¹² and 7.920×10⁻¹² m²/s, respectively.

Now referring to FIG. 5B, the exemplary CO₂ SALLE cluster extraction method comprises three sequential and selective processing steps:

Step 1—CO₂ SALLE Method I: CO₂-L (380);
Step 2—Secondary Infusion: CO₂—S_{S-CO2} (382); and
Step 3—Primary Extraction: CO₂—S_{P-CO2} (384).

In this exemplary CO₂ SALLE method, said cluster extraction processing steps are performed sequentially and selectively using three discrete pressure vessels which are fluidly interconnected using high pressure lines, and facilitated with valves, level sensors, temperature and pressure sensors, liquid substance transfer valve, and at least one dense phase CO₂ pump (all not shown). Further to this, said processing steps may be selectively employed to control the amount of co-extractants delivered into each sequential process step. This aspect is facilitated by high pressure lines and by-pass valves (all not shown).

Step 1—CO₂ SALLE Method I: CO₂-L (380)

In a first step of this exemplary CO₂ SALLE cluster extraction method, an alcoholic beverage (386), an exemplary semi-aqueous solution and liquid substance, containing fermented ethanol and ethanol-soluble organic compounds, is expanded/salted-out and co-extracted using dense phase CO₂ (388) to form a CO₂ salted-out ethanol mixture, the method comprising:
1.1 An alcoholic beverage (386) comprising 100-Proof Vodka (approximately 50% by volume fermented ethanol (i.e., a natural WSWE compound) and 50% by volume water), is placed in a CO₂ SALLE pressure vessel (390);
1.2 Dense phase CO₂ (388) is injected and bubbled (as a near-cryogenic CO₂ gas-solid aerosol) through said alcoholic beverage (386) to cool and saturate with CO₂ to a pre-determined temperature between about 20° C. and −40° C. at a sublimating vapor pressure of about 3 atm (i.e., continuously venting to atmosphere), following which said cooled and CO₂-saturated semi-aqueous solution is autogenously pressurized (vis-à-vis sublimation pressurization and temperature rise) or mechanically pressurized with dense phase CO₂ (388) using a pump (preferred) to a pressure between 5 atm and 90 atm to selectively (and volumetrically) expand/salt-out to form a fermented ethanol-rich CO₂ salted-out solvent mixture (392) phase located above said alcoholic beverage (386) and a liquid CO₂-rich CO₂ salted-out solvent mixture (394) phase located above said ethanol-rich phase (392). The multiphasic mixture thus formed is preferably turbulently mixed and allowed to stratify into two or three distinct phases as shown; and
1.3 A portion of said fermented ethanol-rich CO₂ salted-out solvent mixture (392) is subsequently dissolved (396) into said liquid CO₂-rich phase (394).
   Step 2—Secondary Infusion: CO₂—S_{S-CO}_{₂} (382)

In a second step of this exemplary CO₂ SALLE cluster extraction method, said one or both CO₂ salted-out solvent mixtures (392, 394) from CO₂ SALLE pressure vessel (390) is transferred (398) under CO₂ pressure into a secondary infusion pressure vessel (400) containing a mixture of solid substances to form an infused CO₂ salted-out solvent mixture, the method comprising:
2.1 Said one or both CO₂ salted-out solvent mixtures (392, 394) from CO₂ SALLE pressure vessel (390) is transferred (398) under CO₂ pressure into a secondary infusion pressure vessel (400);
2.2 Said secondary infusion pressure vessel (400) contains one or more secondary solid substances, for example a combination of ground herbs (402) and ground spices (404), which are contained in a porous container (406); and
2.3 Said one or both CO₂ salted-out solvent mixtures (392, 394) penetrate (408) said herbs (402) and spices (404) and extract (410) organic compounds contained therein to form an herb/spice-infused CO₂ salted-out solvent mixture (412).
   Step 3—Primary Extraction: CO₂—S_{P-CO}_{₂}—B (384)

In a third and final step of this exemplary CO₂ SALLE cluster extraction method, herb/spice-infused CO₂ salted-out solvent mixture (412) from the secondary infusion pressure vessel (400) is transferred (414) under CO₂ pressure into a primary extraction pressure vessel (416) containing a primary solid substance to form an herb/spice-infused tincture containing a primary extract, the method comprising:
3.1 Said herb/spice-infused CO₂ salted-out solvent mixture (412) from the secondary infusion pressure vessel (400) is transferred (414) under CO₂ pressure into a primary extraction pressure vessel (416);
3.2 Said primary extraction pressure vessel (416) contains a primary solid substance, for example ground and dried cannabis (418), and is contained in a porous basket (420); and
3.3 Said herb/spice-infused CO₂ salted-out solvent mixture (412) penetrates (422) said cannabis (418) and extracts (424) organic compounds contained therein to form an herb/spice-infused tincture (426) containing a primary extract.

Finally, and still referring to FIG. 5B, said exemplary CO₂ SALLE cluster extraction process is selective. The amount of alcoholic beverage (386) extract and secondary solid substance (402, 404) extract used to form said alcoholic beverage-infused CO₂ salted-out solvent mixtures (392, 394) and said herb/spice-infused CO₂ salted-out solvent mixture (412), respectively, is selectively controlled and used to produce a particular composition of herb/spice-infused tincture (426) containing a primary extract. This is accomplished by selectively transferring dense phase CO₂ (388) through said CO₂ SALLE pressure vessel (390), said secondary infusion pressure vessel (400), and said primary extraction vessel (416) containing a primary solid substance. This is accomplished using three high pressure dense phase CO₂ fluid transfer systems: CO₂ SALLE transfer system (428), secondary infusion transfer system (430), and primary extraction transfer system (432). Said three dense phase CO₂ fluid transfer systems (428, 430, 432) comprise fluidly interconnected high-pressure pipes or lines which are pressurized and filled with dense phase CO₂ using a CO₂ pump and a source of liquid CO₂, and facilitated with high pressure ball valves, check valves, metering valves, and temperature and pressure sensors (all not shown) needed to monitor, control, and direct dense phase CO₂ flow into and through a particular pressure vessel system.

Having described exemplary CO₂ SALLE methods, following is a discussion of a novel hybrid subcritical water-CO₂ SALLE process utilizing a modified heated and pressurized water or semi-aqueous extraction process in combination with a CO₂ SALLE process.

As discussed under FIGS. 3A, 3C, and 3D, tunable extraction systems of the present invention increase the range of phytochemical molecules possessing low-to-high P.S.A., MW, and complexity that can be extracted from plant materials while simplifying and lowering the energy of procedures used to concentrate, desolvate, and recover phytochemical extracts. In this regard, another exemplary type of tunable extraction system is a hybrid extraction system that combines a subcritical water extraction process with a CO₂ SALLE process. A hybrid subcritical water-CO₂ SALLE process provides a full range of cohesion energies ranging from hydrocarbon-like to water-like and permits the use of a full range of process intensification techniques not possible using either extraction method alone. For example, employing higher solvent temperatures to improve cellulosic swelling and solubility of high MW phytochemicals is easily implemented in a water-based solvent system, but not possible using liquid CO₂, nor practical using supercritical CO₂ because of the much higher pressures needed to produce equivalent cohesion energy at higher temperatures. In another example, microwave pretreatment of biomaterials to induce cellular cracking is not practical using a microwave-absorbing semi-aqueous solvent but can be employed in dense phase CO₂ solvent system. In still another example, ultrasonic treatment of biomaterial-liquid solvent mixtures to induce cavitation near solid surfaces to enhance cellular penetration, solvation, and mass transfer of phytochemicals is straightforwardly implemented in a water-based extraction solvent, even at high temperatures, but not practical using a dense phase CO₂ extraction process alone.

Besides serving as a medium for adding beneficial thermal and mechanical energy to a solid-liquid extraction system, a particularly useful aspect of subcritical water is its ability to change cohesion energy based on temperature. As the temperature of water is increased, with an increasing autogenous or artificial vapor pressure to prevent boiling, its cohesion energy is decreased. At a temperature of 300° C. and a vapor pressure of 85 atm, subcritical water exhibits a cohesion energy like an alcohol. As such, subcritical water is a green solvent technology that can provide hydrocarbon-like solvent properties, and which is non-toxic and non-flammable. However, to achieve hydrocarbon-like conditions, a high temperature and pressure must be established. This can be deleterious to phytochemicals and poses high-pressure equipment corrosion and worker safety issues. Moreover, and as discussed herein under prior art, concentrating and recovering extracts is energy intensive and slow. As such, an aspect of the exemplary hybrid subcritical water-CO₂ SALLE process is to lower operating temperatures and pressures needed to achieve full-spectrum solvency. Another aspect of the hybrid extraction process is to simplify and lower energy required to concentrate, desolvate, and recover subcritical water extracts.

A hybrid subcritical water-CO₂ SALLE extraction process uses water-alone or as a WSWE-modified subcritical water solution in a subcritical water extraction process, followed by an exemplary CO₂ SALLE process to concentrate, desolvate, and recover one or more phytochemicals derived from said subcritical water extraction process. For example, a mixture of water and biomaterial is heated and pressurized using N_{2 (g)} or CO_{2 (g)}. One or more WSWE compounds and optional additives may be added to the water prior to heating and performing a primary extraction process, called a modified subcritical water extraction (MSWE) system or process. If CO_{2 (g)} is used in a MSWE process, pressure- and temperature-tunable monophasic or biphasic MSWE extractions may be performed. Alternatively, one or more WSWE compounds and optional additives may be added to an unmodified subcritical water extractant following the primary extraction process and during a CO₂ SALLE process.

Conventional SWE processes employ pressurized N_{2 (g)} to purge dissolved oxygen gas from water to prevent extract oxidation and to provide a vapor pressure to prevent water from boiling at elevated extraction temperatures. By contrast, dense phase CO₂ is employed in the exemplary hybrid subcritical water-CO₂ SALLE process for a variety of useful purposes: (1) a dissolved air purging and gas flotation agent; (2) a vapor pressure control agent; (3) a water acidification agent; (4) a water-ionized and hydrated agent; (5) a dissolved WSWE compound expansion agent; (6) a co-extraction agent; (7) a near-cryogenic sublimation cooling (and CO₂ saturation) agent; and (8) a sublimating desolvation agent.

FIG. 6A is a graph showing the change in cohesion energy in terms of a total Hansen Solubility Parameter (HSP, δ_{T}) versus temperature for three different subcritical water-based solutions. Adding a water-soluble compound such as, for example, ethanol to water forms a so-called hydroethanolic solvent mixture which introduces hydrocarbon-like cohesion energy to the SWE extraction chemistry. Ethanol-water combinations possess broad-spectrum solvent polarities and HSPs capable of extracting a wide range of hydrophilic and lipophilic constituents from a biomass. Still moreover, adding ethanol to water enables a reduction in the subcritical operating temperature (at saturation pressure) required to attain a HSP like propylene carbonate, ethanol, methanol, or acetone, for example. Finally, a hydroethanolic solvent mixture is useful as a feedstock for the CO₂ SALLE process. The presence of dissolved EtOH enables direct implementation of the CO₂ SALLE process.

For example, Plaza et al., FIG. 2, presents an HSP (δ_{T})-Temperature (° C.) curve (“δ_{T}-° C. curve”) for subcritical water, and by comparison to a phytochemical extract called Betulin with a HSP δ_{T}=20.5 MPa^{1/2}, δ_{D}=17.7 MPa^{1/2}, δ_{H}=9.7 MPa^{1/2}, and δ_{P}=3.8 MPa^{1/2}. Betulin is an important triterpene compound commonly extracted from Birch bark, and which has demonstrated efficacy in the treatment of certain types of cancerous tumors. Betulin is insoluble in water (HSP δ_{T} 47.8 MPa^{1/2}), but highly soluble in an alcohol such as ethanol (HSP δ_{T} 26.6 MPa^{1/2}).

As such, in the SWE process, water must be heated (and pressurized) to above 300° C. (according to the δ_{T}-° C. curve under Plaza et al., FIG. 2, and reproduced under FIG. 6A as 100% water (δ_{T}-° C. curve) to attain an Betulin-like HSP. However, operating a SWE process at elevated temperature and pressure, and particularly for an extended period, can cause oxidation and denaturing of labile phytochemicals.

Now referring to FIG. 6A, adapting the δ_{T}-° C. curve under Plaza et al., FIG. 2, for 100% water (440) using Teas fractional cohesion parameters (Hansen 2007) and plotting δ_{T}-° C. curves for a 80:20 (Water:EtOH v:v) mixture (442) and a 50:50 (Water:EtOH v:v) mixture (444) reveals that the needed operating temperatures and vapor pressures for a modified subcritical water extractant are significantly lower to attain an equivalent and optimal HSP δ_{T} value of 25 MPa^{1/2}; from 300° C. at 85 atm (446), to 225° C. at 25 atm (448), and to 175° C. at 9 atm (450), respectively. Vapor pressures for each temperature are for (pure) water and were determined using the Antoine equation with appropriate constants for the subcritical range values plotted.

In this regard, and still referring to FIG. 6A, the beneficial reduction in operating temperature and corresponding vapor pressure for a hybrid extraction process is due to a reduction in the total cohesion energy (δ_{T}) (452) of the solvent extraction system. The reduction in total cohesion energy (452) is due to (and selectively controlled by) both a thermal energy effect (454) and a chemical energy effect (456). The thermal effect (454) is caused by the heating of the modified subcritical water extractant. The chemical effect (456) is caused by the addition of lower cohesion energy WSWE compounds (i.e., ethanol) into water, and if used with dense phase CO₂, CO₂ hydration by water and CO₂ expansion of said WSWE compounds dissolved in water. The thermal effect (454) introduces molecular vibrational (kinetic) energy that disrupts (decreases) hydrogen bonding. The chemical effect (456) introduces molecular ionization or complexation, and molecular expansion that disrupts (decreases) both hydrogen bonding and polarity. As such, the thermal effect (454) predominantly affects the δ_{H} component of the total cohesion energy (452) and the chemical effect (456) affects both the δ_{H} and δ_{P} of the total cohesion energy (452).

FIG. 6B is a chart contrasting and comparing the change in total cohesion energy versus semi-aqueous solution temperature for a modified subcritical water extraction (MSWE) of FIG. 6A with integration of an exemplary CO₂ SALLE process of FIG. 3B. Now referring to FIG. 6B, HSP δ_{T}-° C. curve (FIG. 6A, (444)) represents the change in total cohesion energy (FIG. 6A, (452)) over a temperature range of between 25° C. to 275° C. for an exemplary 50%:50% H₂O:EtOH semi-aqueous mixture used as a modified subcritical water extraction (MSWE) solution. As shown in FIG. 6B, the HSP δ_{T}-° C. curve (FIG. 6A, (444)) spans a total cohesion energy (FIG. 6A, (452) between approximately 36 MPa^{1/2} (460) to approximately 13 MPa^{1/2} (462) for this same temperature range under a vapor pressure ranging between approximately 1 atm and 60 atm.

It is well established in the prior art that optimal extraction efficiency is attained using a conventional SWE process at a temperature of 150° C. or less, and an extraction time of 30 minutes or less. For example, Saim, N. et al., “Subcritical Water Extraction of Essential Oils from Coriander (Coriandrum sativum L.) Seeds”, The Malaysian Journal of Analytical Sciences, Vol. 12, No. 1, 2008 (Saim et al.) investigated the use of SWE in the extraction of essential oil from coriander (Coriandrum sativum L.) seeds. Ground coriander seeds were subjected to SWE with water for an extraction time of 15 min under several extraction conditions comprising vapor pressures of 60 atm and 70 atm and temperatures of 65, 100 and 150° C. Saim et al. compared the SWE method extraction efficiency with another water-based extraction technique called hydrodistillation, a process that requires approximately 3 hours to complete. Extracted compounds dissolved in water-based extractants from the SWE method and hydrodistillation method were extracted with hexane and determined by gas chromatography mass spectrometry (GC-MSD). Saim et al. determined that the efficiency (g oil/g of coriander) of SWE was higher than that provided by hydrodistillation with reduced extraction time. The major compounds found were linalool, isoborneol, citronellyl, butyrate, and geraniol. Further to this, Saim et al. determined that the SWE method has the possibility of manipulating the composition of the oil by varying the temperature and adjusting the pressure. Moreover, vapor pressure was found to be an unimportant key process variable (KPV). SWE temperature was determined to be the main driver. This is indicative of the heating effect on decreasing hydrogen bonding energy (C) to produce an extraction chemistry with a lower cohesion energy. Based on FIG. 6A, at 150 C, an unmodified subcritical water extraction solvent (FIG. 6A, (440)) exhibits a total cohesion energy (FIG. 6A, (452) of approximately 40 MPa^{1/2}. Also significant, Saim et al. determined that an extraction temperature greater than (>) 150° C. resulted in extract losses due to volatilization as well as chemical and thermal degradation. At a lower extraction temperature of 65° C. and an extraction time of 15 min, the extraction yield was superior to SWE performed at elevated temperatures and to the conventional hydrodistillation method performed for 3 hours.

Finally, the investigation of Saim et al. showed that the thermal effect (FIG. 6A, (454) is the key driver in a conventional SWE process. Just a small reduction in total cohesion energy, from 47.8 MPa^{1/2} at 20° C. to 40 MPa^{1/2} at 150° C., an 8 MPa^{1/2} total cohesion energy difference, provides a remarkably high extraction efficiency. As such, this would indicate that the thermal effect probably involves several synergistic effects besides hydrogen bonding energy reduction, for example cellulosic swelling actions (improved solvent access and mass transfer) and critical solution temperature (CST) effects (complete extract solubility at high temperature). In this regard, an aspect of the present invention is that increasing CO₂ pressure lowers the CST of the extraction solvent system vis-à-vis CO₂ expansion and salting-out of the WSWE components. Again, referring to FIG. 6B, a hybrid subcritical water-CO₂ SALLE process enables the use of lower extraction temperatures while expanding the total cohesion energy (FIG. 6A, (452)). An exemplary 50%:50% H₂O:EtOH v:v semi-aqueous mixture (464) at 25° C. and a N_{2 (g)} vapor pressure of 10 atm has a HSP δ_{T} of about 36 MPa^{1/2} (460). Heating said semi-aqueous mixture (464) to a temperature of 150° C. (466) and injecting CO_{2 (g)} to establish a vapor pressure of 20 atm produces a monophasic solution with a HSP δ_{T} of about 27 MPa^{1/2} (468). Following this, cooling and pressurizing said heated solution (466) with dense phase CO₂ to a temperature of 25° C. and to a CO₂ pressure of 80 atm (470) produces a biphasic or multiphasic solution. The production of a biphasic or multiphasic solution depends upon the volume of ethanol-extract expanded/salted-out solvent phase produced from the semi-aqueous solution and the volume of liquid CO₂ produced. For example, an excess amount of ethanol-extract phase saturates a smaller volume of liquid CO₂ phase and results in a multiphasic solution comprising a water-rich semi-aqueous solution (lower phase), an ethanol-rich CO₂ salted-out solvent mixture (middle phase), and a liquid CO₂-rich CO₂ salted-out solvent mixture (upper phase). The CO₂ salted-out solvent mixtures containing CO₂ expanded ethanol, extracts, and CO_{2 (g/l)} have an HSP δ_{T} as low as 15 MPa^{1/2} (472). Given this, a modified subcritical water extraction (MSWE) process (474) combined with a CO₂ SALLE process provides a more robust extraction process with a temperature range of between 25° C. and 150° C., with a HSP δ_{T} range between about 36 MPa^{1/2} and 18 MPa^{1/2}, an 18 MPa^{1/2} total cohesion energy difference. In summary, the hybrid MSWE-CO₂ SALLE process optimizes thermal and chemical energy to provide full spectrum solvency.

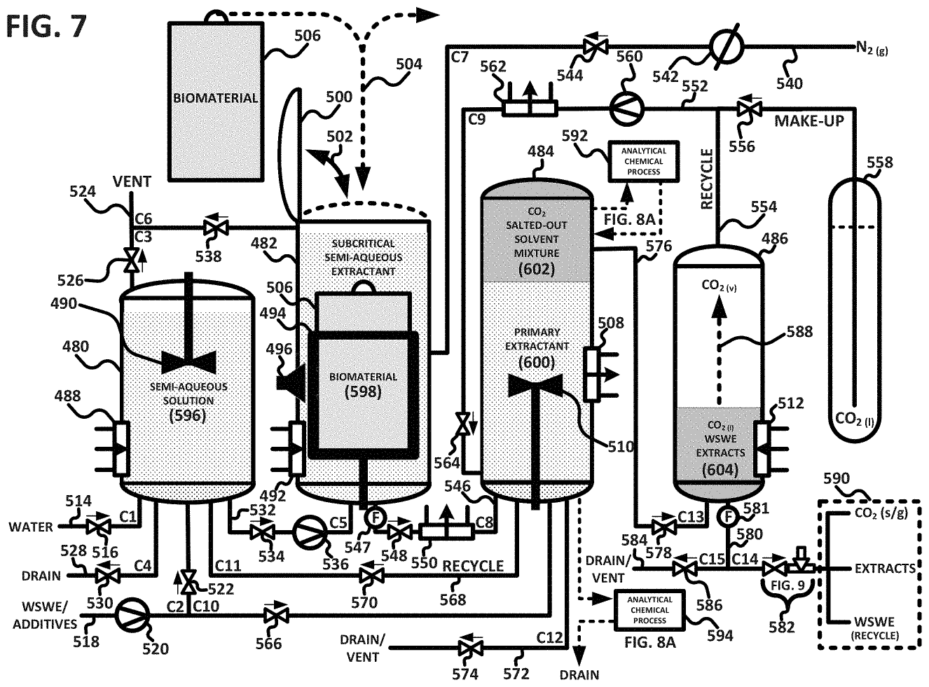

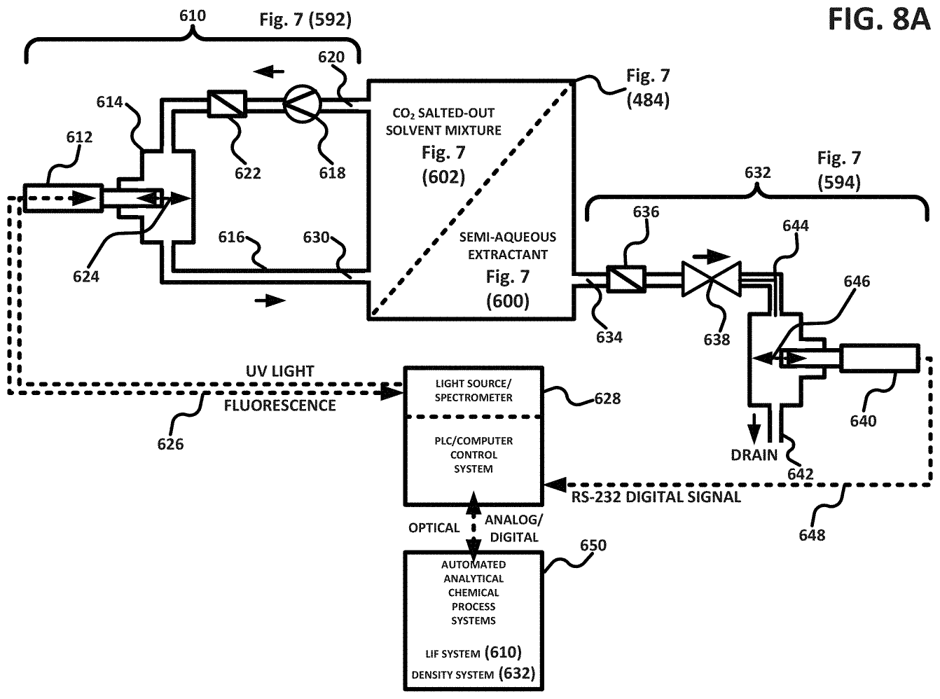

A solid-liquid phase extraction utilizing said hybrid MSWE-CO₂ SALLE process can be performed sequentially and in-situ using a single pressure vessel system. However, a single pressure vessel system is mainly useful for R&D systems without time and capacity constraints. More preferably, and for high-capacity extraction applications, multiple pressure vessel systems are used in sequence to optimize time, energy, extraction capacity, and extract and extraction media recovery operations. An exemplary multiple vessel processing system is described under FIG. 7 herein. For example, subcritical water extractant produced in a heated-pressurized MSWE pressure vessel system is transferred under gas pressure, cooled in transit, to a CO₂ SALLE pressure vessel system which provides biphasic or multiphasic extraction and CO₂-WSWE-extract recovery operations. The extracted solid substance contained in the MSWE pressure vessel system is discarded or transferred to a depressurized CO₂ SALLE pressure vessel system to perform a secondary biphasic or multiphasic solid-liquid extraction process, followed by extract concentration, desolvation, and recovery operations.

FIG. 7 provides a schematic of an exemplary hybrid MSWE-CO₂ SALLE system. Now referring to FIG. 7, the exemplary hybrid system comprises four pressure vessel subsystems, from left to right:
1. Semi-aqueous solution pressure vessel subsystem (480), rated for a maximum operating temperature of 100° C. and a maximum pressure of 10 atm;
2. MSWE pressure vessel subsystem (482), rated for a maximum operating temperature of 150° C. and a maximum pressure of 60 atm;
3. CO₂ SALLE pressure vessel subsystem (484), rated for a minimum operating temperature of −40° C., a maximum operating temperature of 100° C., and a maximum pressure of 80 atm; and
4. Desolvation pressure vessel subsystem (486), rated for a minimum operating temperature of −20° C., a maximum operating temperature of 100° C., and a maximum operating pressure of 80 atm.

Said pressure vessel subsystems are designed and constructed using materials suitable for operating at the exemplary temperatures and pressures and employing CO₂ and water-based process fluids of the present invention. In this regard, stainless steel or Hastelloy, Haynes® high performance alloys, are preferred materials of construction.

The semi-aqueous solution pressure vessel subsystem (480) is thermally insulated and equipped with a heating means (488) such as a steam heat exchanger, a thermostatically regulated electric band heater, or a recirculating fluid heater system capable of preheating the subsystem and semi-aqueous solution content to a maximum temperature of about 100° C., and a mixing means (490) such as a magnetically-driven mixing blade or a recirculating fluid heater-in-line static mixing system.

The MSWE pressure vessel subsystem (482) is thermally insulated and equipped with a heating means (492) such as a thermostatically regulated electric band heater or a recirculating fluid heater system capable of heating the subsystem and contents to an extraction temperature between 50° C. and 150° C., a mixing means comprising a magnetically-driven bladed centrifuge drum (494) with a torque as needed to rotate a centrifuge basket and biomaterial content, while mixing with a semi-aqueous subcritical extractant, and a titanium ultrasonic horn (496) with an energy capacity as needed to sonicate subsystem contents contained in said centrifuge drum (494). Said MSWE pressure vessel subsystem is further equipped with a quick-opening closure (500) which can be conveniently opened and closed (502) to insert and withdrawal (504) a centrifuge basket (506) containing a dried or dewatered biomaterial.

The CO₂ SALLE pressure vessel subsystem (484) is thermally insulated and equipped with a cooling means (508) such as a chilled-water heat exchanger or a recirculating refrigerated fluid cooling system capable of cooling the subsystem and contents to between −40° C. and 30° C., a mixing means (510) such as a magnetically driven mixing blade or a recirculating refrigerated fluid cooler-in-line static mixing system.

Finally, the desolvation pressure vessel subsystem (486) is not thermally insulated and is equipped with heating means (512) such as a thermostatically regulated electric band heater circumferentially affixed to the surface of the pressure vessel near the lower hemisphere, and capable of heating the lower section of the subsystem and contents to produce a clean CO_{2 (g)} distillate temperature between 20° C. and 40° C.

Having described exemplary features, following is a discussion of high-pressure fluid interconnections and process fluid supply connections between and into the exemplary pressure vessel subsystems. The exemplary pressure vessel subsystems thus described are fluidly interconnected to each other and to external process fluid supplies including water, WSWE compounds and additives, nitrogen gas, and liquid carbon dioxide, referred to as “circuits” herein. Moreover, each subsystem is fluidly connected to either a venting and/or draining circuit, for a total of fifteen fluid transfer circuits (C1-C15).

Again, referring to FIG. 7, the semi-aqueous solution pressure vessel subsystem (480) is fluidly interconnected to a source of pressurized water through high pressure supply line or pipe (514) and water inlet supply valve (516); collectively referred to as the “water supply circuit (C1)” herein. In addition, the semi-aqueous solution pressure vessel subsystem (480) is fluidly interconnected to a source of WSWE and additives through high pressure supply line or pipe (518), WSWE supply pump (520), and WSWE inlet supply valve (522); collectively referred to as the “semi-aqueous solution WSWE supply circuit (C2)” herein. Moreover, the semi-aqueous solution pressure vessel subsystem (480) is fluidly interconnected to the atmosphere through a high-pressure vent line or pipe (524) and atmospheric vent valve (526); collectively referred to as the “semi-aqueous solution atmospheric vent circuit (C3)” herein. Still moreover, the semi-aqueous solution pressure vessel subsystem (480) is fluidly interconnected to a drain using a high pressure drain line or pipe (528) and drain valve (530); collectively referred to as the “semi-aqueous solution drain circuit (C4)” herein. Finally, the semi-aqueous solution pressure vessel subsystem (480) is fluidly interconnected to the MSWE pressure vessel subsystem (482) using a high pressure semi-aqueous fluid transfer line or pipe (532), liquid transfer pump (534), and semi-aqueous solution inlet supply valve (536); collectively referred to as the “semi-aqueous solution supply circuit (C5)” herein.

The MSWE pressure vessel subsystem (482) is fluidly interconnected to the atmosphere through a high-pressure vent line or pipe (524) and atmospheric vent valve (538); collectively referred to as the “MSWE subsystem atmospheric vent circuit (C6)” herein. Moreover, the MSWE pressure vessel subsystem (482) is fluidly interconnected to a source of regulated nitrogen gas through a high-pressure line or pipe (540), nitrogen pressure regulator (542), and nitrogen gas inlet valve (544); collectively referred to as the “nitrogen gas supply circuit (C7)” herein. Finally, the MSWE pressure vessel subsystem (482) is fluidly interconnected to the CO₂ SALLE pressure vessel subsystem (484) using a high pressure semi-aqueous fluid transfer line or pipe (546), fluid filter (547), subcritical water extractant inlet supply valve (548), and cooling heat exchange means (550); collectively referred to as the “subcritical water extractant supply circuit (C8)” herein.

The CO₂ SALLE pressure vessel subsystem (484) is fluidly interconnected to a source of recycled or make-up CO₂ supply through a high pressure CO₂ inlet line or pipe (552) connected to the upper hemisphere (554) of the desolvation pressure vessel subsystem (486), and through high pressure CO₂ inlet valve (556) and high pressure liquid CO₂ supply (558), CO₂ gas-liquid transfer pump (560), cooling heat exchanger means (562), and liquid CO₂ inlet valve (564); collectively referred to as the “CO₂ supply circuit (C9)” herein. Moreover, the CO₂ SALLE pressure vessel subsystem (484) is fluidly interconnected to a source of WSWE and additives through high pressure supply line or pipe (518), WSWE supply pump (520), and WSWE inlet supply valve (566); collectively referred to as the “CO₂ SALLE WSWE supply circuit (C10)” herein. Still moreover, the CO₂ SALLE pressure vessel subsystem (484) is fluidly interconnected to the WSWE pressure vessel system (482) through high pressure solution recycle line or pipe (568) and solution recycle valve (570); collectively referred to as the “raffinate recycle circuit (C11)” herein. In addition, the CO₂ SALLE pressure vessel subsystem (484) is fluidly interconnected to the drain through high pressure CO₂ SALLE solution drain line or pipe (572) and CO₂ SALLE solution drain valve (574); collectively referred to as the “CO₂ SALLE drain circuit (C12)” herein. Finally, the CO₂ SALLE pressure vessel subsystem (484) is fluidly interconnected to the desolvation pressure vessel system (486) through high pressure CO₂ salted-out solvent mixture line or pipe (576) and CO₂ salted-out solvent mixture valve (578); collectively referred to as the “CO₂ salted-out solvent mixture supply circuit (C13)” herein.

The desolvation pressure vessel subsystem (486) is fluidly interconnected to high pressure CO₂ inlet line or pipe (552) through the upper hemisphere (554) of the desolvation pressure vessel subsystem (486); collectively referred to as the “CO₂ supply circuit (C9)” herein. Moreover, the desolvation system is fluidly interconnected to the near-cryogenic desolvation system through high pressure CO₂-WSWE-Extract desolvation line or pipe (580), fluid filter (581), and desolvation device (582), discussed in more detail under FIG. 9; collectively referred to as the “near-cryogenic desolvation circuit (C14)” herein. Still moreover, the desolvation pressure vessel subsystem (486) is fluidly interconnected to drain through high pressure CO₂ drain line or pipe (584) and CO₂ drain valve (586); collectively referred to as the “desolvation subsystem drain circuit (C15)” herein. Finally, desolvation processes used in the exemplary desolvation pressure vessel subsystem (486) comprises isostatic (high pressure) CO₂ distillation (588) and near-cryogenic capillary condensation (590). Moreover, and not shown, many other desolvation methods and processes may be used to process the concentrated CO₂ salted-out solvent mixture (604) to separate, recover, and recycle water-based extractant, water-soluble or water-emulsifiable compounds, dense phase carbon dioxide, and extract. Exemplary desolvation methods include gravity separation, phase separation, near-cryogenic phase separation, high pressure distillation, atmospheric distillation, vacuum distillation, membrane separation, gas flotation, or evaporation.

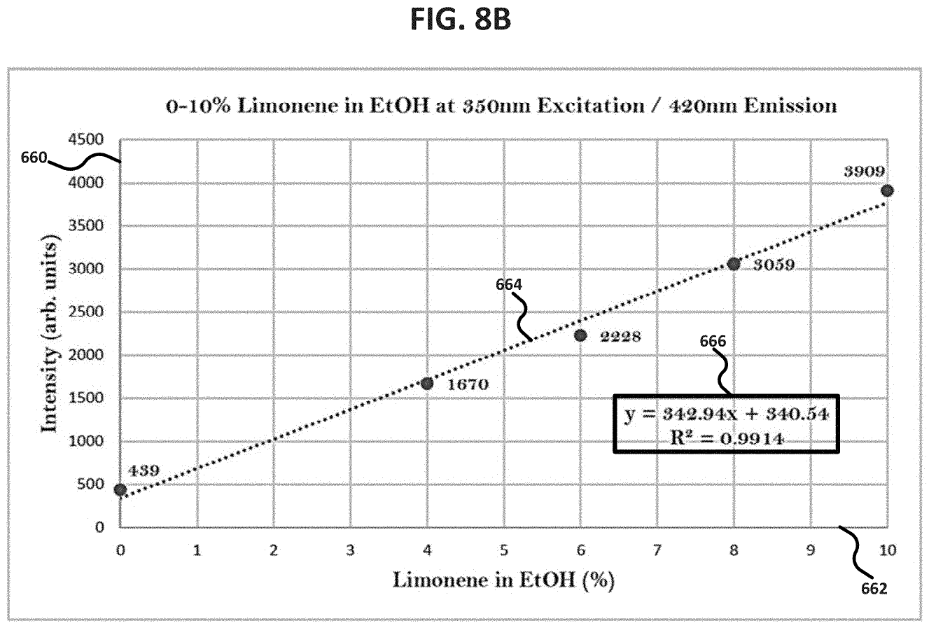

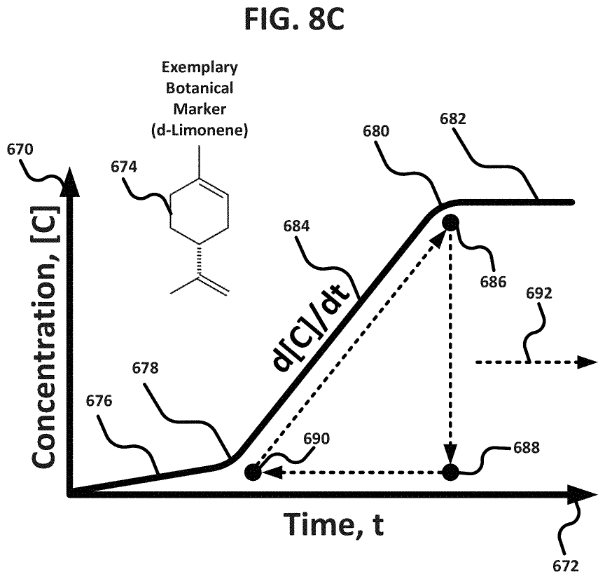

Finally, the exemplary hybrid MSWE-CO₂ SALLE system shown in FIG. 7 is preferably automated and controlled by a process logic controller (PLC) and software integrated with variously located temperature, pressure, and level sensors (all not shown), and with said transfer pumps, automated valves, heating means, and cooling means described herein. Moreover, exemplary analytical chemical processes, described in more detail under FIG. 8A herein, may be employed to monitor KPVs of the CO₂ SALLE process. For example, selective measurement of a key extract chemical marker (592) dissolved within the WSWE-rich or liquid CO₂-rich CO₂ salted-out solvent mixtures (non-aqueous phase) or measurement of relative solution density (594) to determine the concentration level of dissolved WSWE-additive compounds in the semi-aqueous solution (aqueous phase) as the CO₂ expansion/salting-out and desolvation process progresses.

Still referring to FIG. 7, an exemplary MSWE-CO₂ SALLE method comprises the following steps and processes:

### Biomaterial Preparation Process

A dry biomaterial is ground using a grinder to between about 0.5- and 2-mm particle size using a conventional grinder and poured into a semi-permeable or porous container constructed from a non-contaminating material. Following this, the container of ground biomaterial is placed into a centrifuge basket (506).

### Semi-Aqueous Solution Preparation Process

A predetermined amount of water is introduced into the semi-aqueous solution pressure vessel subsystem (480) through fluid transfer circuit (C1). With the fluid mixing means (490) operational, a predetermined amount of WSWE compound and optional additives is introduced through fluid transfer circuit (C2) and mixed into the water. The mixture is heated using the fluid heating means (488) to a predetermined temperature, for example 80° C., to form a heated semi-aqueous solution (596) therein.

### MSWE Pressure Vessel Subsystem Preparation Process

The closure (500) of the MSWE pressure vessel system (482) is opened (502), following which the centrifuge basket (506) containing pre-ground biomaterial is transferred (504) and placed into the internal centrifuge drum (494). The closure (500) of the MSWE pressure vessel system (482) is closed (502).

### Modified Subcritical Water Extraction Process

The heated semi-aqueous solution (596) contained in the semi-aqueous solution pressure vessel subsystem (480) is pumped into the MSWE pressure vessel subsystem (482) through fluid transfer circuit (C5). Following this, MSWE atmospheric vent valve (538) is opened, and nitrogen gas is introduced through fluid transfer circuit (C7) for a predetermined amount of time at a pressure of 2 atm to remove dissolved oxygen from the heated solution. Following this, nitrogen gas flow is stopped, and with the MSWE atmospheric vent valve (538) still open, the ultrasonic treatment horn (496) is energized for a predetermined amount of time and power level, during which the centrifuge drum is slowly rotated to thoroughly sonicate and degas the biomaterial and solution (598), respectively. Following sonication and degas operations, the MSWE atmospheric vent valve (538) is closed, and nitrogen gas is introduced again through fluid transfer circuit (C7) to provide a suitable internal vapor pressure necessary to prevent solution boiling at operating temperature. For example, the fluid heating means (492) is used to increase the temperature of the semi-aqueous solution (extractant) from 80° C. to 125° C. with a N₂ (g) vapor pressure of 10 atm. During the heating cycle the centrifuge drum is rotated at a predetermined speed, for example between 10 and 100 rpm. Upon reaching the predetermined extraction temperature (and pressure), the heated, pressurized, and dynamic extraction system is maintained for a predetermined amount of time, for example between 15 and 240 minutes. The extraction time is dependent upon the extraction solution temperature, chemical composition of the semi-aqueous solution, and the type and concentration of target phytochemicals. Following completion of the MSWE process, the centrifuge drum is slowed and the primary extractant (600) containing biomaterial extracts is transferred using the N_{2 (g)} gas pressure, filtered, and cooled in transit through fluid transfer circuit (C8) into the CO₂ SALLE pressure vessel subsystem (484). Following transfer of the primary extractant (600), residual N_{2 (g)} gas pressure is removed from the MSWE subsystem (482) through MSWE vent circuit (C6). Upon reaching atmospheric pressure, the closure (500) is opened and the centrifuge basket (506) containing the extracted biomaterial is removed from the centrifuge drum and transported (504) to a discard and refill station (not shown).

### CO₂ SALLE Process

Pre-cooled primary extractant (600) is further cooled to a predetermined temperature below 30° C. using the cooling means (508), during which mixing means (510) is operating. During cool down and mixing operations, liquid CO₂ is introduced into the CO₂ SALLE subsystem (484) through fluid transfer circuit (C9) to produce a predetermined dense phase CO₂ pressure, for example in discrete stages, between 20 atm and 80 atm, which initiates and progresses the CO₂ expansion and salting-out assisted liquid-liquid extraction process. Liquid CO₂ mixes with the primary extractant (600) to cool and saturate with CO₂, which expands dissolved WSWE compounds and forms aqueous CO₂. Once the desired final CO₂ fluidization pressure is reached, the mixing means (510) is stopped to allow the mixture to separate into distinct phases. The exemplary CO₂ SALLE process produces a biphasic stratification: a lower water-rich primary extractant phase (600), a predominantly aqueous phase, and an upper liquid CO₂-rich CO₂ salted-out solvent mixture (602), a predominantly non-aqueous phase, containing a dissolved portion of WSWE and extracts phase-separated from the primary extractant (600).

Following this, a predetermined amount of the liquid CO₂-rich CO₂ salted-out solvent mixture (602) is withdrawn from the CO₂ SALLE subsystem and transferred to the desolvation subsystem (486) through fluid transfer circuit (C13) for extract concentration, desolvation, and recovery operations. As already discussed herein, the CO₂ SALLE process may be monitored using an analytical chemical process, for example using in-situ instrumental analysis of the non-aqueous or aqueous phases discussed under FIG. 8A.

### Desolvation Process

Liquid CO₂-rich CO₂ salted-out solvent mixture (602) withdrawn from the CO₂ SALLE subsystem, comprising a concentrated mixture of liquid CO₂, WSWE/additives, and solvated or desolvated extracts (604), is heated using heating means (512). Heating the concentrated mixture (604) to between about 25° C. and 40° C. distills out high pressure CO₂ gas (588), which is withdrawn (554), compressed (560), and condensed (562) into a pure liquid CO₂ co-extractant for reuse in the CO₂ SALLE subsystem (484) through fluid transfer circuit (C9). This withdrawal-recycle sequence is repeated as required to completely dissolve and withdraw the CO₂ salted-out solvent mixture from the CO₂ SALLE subsystem (484), which further concentrates the mixture (604) contained in the desolvation subsystem (486). Finally, the CO₂-extracted primary extractant (600) contained in CO₂ SALLE subsystem (484), and now depleted of WSWE compounds and biomaterial extracts, is transferred under CO₂ pressure back to the original semi-aqueous solution pressure vessel subsystem (480) through fluid transfer circuit (C11). The recycled extractant may be reformulated with new or recycled WSWE compounds from the desolvation process. Alternatively, the CO₂-extracted primary extractant (600) is transferred under CO₂ pressure to the drain through fluid transfer circuit (C12).

### Extract Recovery and Process Fluids Recycling Process

The concentrated mixture (604) containing CO₂-WSWE-Extracts is removed under CO₂ gas pressure though fluid transfer circuit (C14). An exemplary desolvation and separation process (590) discussed herein uses a near-cryogenic CO₂ (s→g) aerosol assembly and process described under FIG. 9 to expand and desolvate the extracts and WSWE compounds, and separate crystallized CO₂ particles as a sublimated gas back into the atmosphere. This atmospheric desolvation process produces mixture temperatures as low as −77 C, which prevents volatilization of low MW extracts. Following this, the WSWE-extract mixture may be distilled to produce an extract mixture and recyclable WSWE compounds.

### Selectivity

Finally, there are numerous possible solid-liquid and liquid-liquid extraction system and process schemes and configurations utilizing the hybrid subcritical water-CO₂ SALLE process. A novel aspect of the present invention is the ability to be selective (i.e., produce select fractions of a particular polarity of phytochemical extracts) or non-selective (i.e., produce a full spectrum of mixed polarity extracts). An example of a selective process follows. Subcritical water extractant (FIG. 7, (598)) used without a WSWE compound or optional additives will extract and dissolve both polar and nonpolar extracts from plant materials, and particularly at higher extraction temperatures under which unmodified water chemically behaves like ethanol. As discussed under FIGS. 6A and 6B, this is due to the thermal energy effect which progressively reduces water hydrogen bonding cohesion energy as temperature increases. However, once the heated and pressurized water-only extractant containing dissolved nonpolar extracts is introduced into the CO₂ SALLE subsystem (FIG. 7, (484)), and cooled, the solvated nonpolar extracts will desolvate due to a huge increase in the cohesion energy of the water-based extractant to form a nonpolar CO₂-soluble phase, like a CO₂ salted-out WSWE phase. Polar water-soluble plant extracts will remain dissolved in the water phase. Following removal of the non-polar extract fraction using the dense phase CO₂ solvent mixture withdrawal and desolvation processes described under FIG. 7, WSWE compounds and additives may be added to water-based extractant. Alternatively, the hot and pressurized water-based extractant may be decanted to a separate cooling-separation tank (not shown) whereupon the desolvated nonpolar extracts (i.e., plant oils) are desolvated and separated using in-situ dissolved gas flotation (nitrogen or carbon dioxide degassing) and recovered from the surface of the cooled-degassed extractant using a commercial oil skimmer device, for example, available from Abanake Corporation, Chagrin Falls, Ohio, USA. Following this, the cooled and oil skimmed extractant is returned to the CO₂ SALLE subsystem (FIG. 7, (484)) for dissolved polar extract recovery. WSWE compounds and additives are added to the cooled and oil skimmed extractant, and the CO₂ SALLE process is performed to expand/salt-out WSWE compounds containing water-soluble extracts using the same dense phase CO₂ co-extraction and desolvation processes described under FIG. 7.

In summary, an exemplary semi-aqueous extraction method using the apparatus and process described under FIG. 7 comprises a semi-aqueous extraction method for recovering an extract from a natural product, the steps comprising:
1. Placing the natural product containing the extract into a first pressure vessel (FIG. 7, (482));
2. Adding a semi-aqueous solution, which comprises water and a water-soluble or water-emulsifiable compound, to the first pressure vessel (FIG. 7, 480));
3. Pressurizing said semi-aqueous solution and natural product with dense phase CO₂ to a pressure between 1 atm and 340 atm to establish a tunable extraction system within the first pressure vessel (FIG. 7, 482));
4. Heating said tunable extraction system contained within the first pressure vessel to a temperature between 30° C. and 300° C. and maintaining temperature for a time between 5 minutes and 120 minutes to produce a heated water-based extractant containing water-soluble or water-emulsifiable compound and extract within the first pressure vessel (FIG. 7, (482));
5. Cooling said heated water-based extractant to a temperature between −40° C. and 40° C. during transfer to a second pressure vessel (FIG. 7, 484));
6. Expanding and salting-out said cooled water-based extractant within the second pressure vessel using dense phase CO₂ to produce a first separated phase, which comprises water-soluble or water-emulsifiable compound containing the extract (FIG. 3B, (152));
7. Simultaneously co-extracting said first separated phase in the second pressure vessel with said dense phase CO₂ to produce a second separated phase, which comprises a CO₂ salted-out solvent mixture containing the extract (FIG. 3B, (154));
8. Transferring said CO₂ salted-out solvent mixture containing the extract to a third pressure vessel (FIG. 7, (486)); and
9. Desolvating said CO₂ salted-out solvent mixture within the third pressure vessel to concentrate and recover said extract (FIG. 7, (590)).

The natural product can comprise plant, vegetable, fruit, nut, spice, herb, hops, root, bark, hemp, or cannabis; and said extract is decarboxylated.

Having described exemplary aspects of a hybrid subcritical water-CO₂ SALLE extraction process under FIG. 7 as well as several alternative and novel extraction schemes possible utilizing a CO₂ SALLE process, following is a discussion of exemplary analytical chemical processes used to monitor key process variables of the CO₂ SALLE process under FIGS. 8A, 8B, and 8C.

In-situ analytical chemical processes are used herein to provide real-time direct or indirect measurement of so-called marker chemicals (key extracts) and WSWE compounds during a CO₂ SALLE process. Analyzing CO₂ salted-out solvent mixtures and/or semi-aqueous extractants provides useful information about the condition and progress of the CO₂ SALLE system and process, respectively. In the following discussion, an exemplary analytical technique called light-induced fluorescence (LIF) spectroscopy is used to measure changes in concentration of a dissolved terpene, d-limonene, contained in the CO₂ salted-out solvent mixture during an exemplary botanical solid-liquid CO₂ SALLE process. Related to this, the relative density of the semi-aqueous extractant is measured to monitor the progress of the WSWE salting-out process. Moreover, although the present example uses a solid botanical material in the exemplary solid-liquid CO₂ SALLE process described under FIG. 7, the same analytical chemical process techniques may be used in virtually any solid-liquid and liquid-liquid extraction application utilizing the CO₂ SALLE process, for example as described under FIG. 4 herein.

FIG. 8A is a schematic showing the integration and use of exemplary in-situ analytical process chemical systems for monitoring and controlling KPVs of the CO₂ SALLE process during a solid-liquid extraction process, for example extracting terpenes and cannabinoids from hemp flowers. Now referring to FIG. 8A, an exemplary analytical chemical process technique for monitoring the CO₂ salted-out solvent mixture (FIG. 7, (602)) produced in the CO₂ SALLE process uses a closed-loop light-induced fluorescence (LIF) probe and spectroscope integrated with a recirculating fluid sampling and measurement capillary loop, referred to herein as the LIF system (610), as shown in FIG. 7 (592). The LIF system (610) comprises an LIF optical probe (612) affixed to a high-pressure optical measurement tee (614) and high pressure PEEK capillary sampling loop (616). A small high-pressure pump (618) withdrawals CO₂ salted-out solvent mixture (FIG. 7, (602)) through an inlet sample capillary tube (620), continuously or periodically during the CO₂ SALLE process, and transports the fluid sample through a fluid filter (622) and through said optical measurement tee (614). A LIF measurement (624) is obtained within said optical measurement tee (614), which is optically communicated (626) to a spectroscope and PLC/computer system (628). Finally, the processed sample is returned to the bulk CO₂ salted-out solvent mixture (FIG. 7, (602)) through an outlet sample capillary tube (630).

The LIF optical probe (612) is used to measure the concentration of key “chemical markers” dissolved within the CO₂ salted-out solvent mixture (FIG. 7, (602)) during the CO₂ SALLE process. During a continuous or periodic sampling and measurement process, the LIF optical probe (612) simultaneously emits light while measuring a resulting fluorescence (624) of one or more organic compounds. Organic compounds that fluoresce when exposed to ultraviolet light are termed “fluorophores” and most unsaturated botanical compounds are fluorophores. Botanical fluorophores dissolved in the CO₂ salted-out solvent mixture (FIG. 7, (602)) are selectively excited to a higher energy state by the absorption of light in the range between 200 nm to 2200 nm, which is then followed by a near-spontaneous re-emission of light. The wavelength is selected to be the one at which the botanical species has its largest cross section. Usually within a few nanoseconds to microseconds, the excited botanical species de-excite and emit light at a wavelength longer than the excitation wavelength.

Use of LIF spectroscopy in botanical extractions is well established. For example, LIF spectroscopy is used in CBD fractional distillation processes to determine the quality of a distillate fraction. In Ranzan, C. et al., “Fluorescence Spectroscopy as a Tool for Ethanol Fermentation On-line Monitoring”, 8th IFAC Symposium on Advanced Control of Chemical Processes, Furama Riverfront, Singapore, Jul. 10-13, 2012 (Ranzan et al.), Ranzan et al. details a fluorescence spectroscopy process and system for monitoring and controlling bio-based ethanol production. LIF spectroscopy is used to monitor the progress of the fermentation process based on time-based changes in sucrose, ethanol, biomass, and glycerol concentration within a fermentative broth.

Another exemplary analytical chemical process is a relative density measurement. An exemplary relative density measurement system uses an open-loop density sensor integrated with a fluid sampling and measurement capillary delivery line, referred to herein as the relative density system (632), as shown in FIG. 7 (594). The relative density system (632) comprises an inlet capillary tube (634), fluid filter (636), fluid sample inlet valve (638), density sensor (640), and an outlet capillary tube (642), which is connected to a drain. A microscopic PEEK capillary tube (644), for example with an internal diameter of 0.008 inches, is connected to the outlet of the sampling valve (638) to provide a low pressure, low flow fluid transfer through the density sensor (640). Moreover, preferably a full-mixed biphasic sample of the semi-aqueous extractant (FIG. 7, (594) is withdrawn through capillary tube (634), degassed to remove excess CO₂ and to allow remaining phase-separated WSWE-additives to re-dissolve into solution, and then analyzed to determine a relative density.

The relative density system (632) is used to periodically sample and measure the relative density of the semi-aqueous extractant (FIG. 7, (600)) to determine the relative concentration of WSWE-additive compounds remaining in the semi-aqueous extractant during the CO₂ SALLE process. The density sensor (640) contains a microelectromechanical system (MEMS) that measures (646) a precise small volume and low-pressure sample of degassed and homogeneous semi-aqueous extractant (FIG. 7, (594)). Subsequently, the internal MEMS device transmits a density value between 0.6 g/ml and 1 g/ml vis-à-vis a RS232 interface (648) to the PLC/computer system (628).

Finally, and again referring to FIG. 8A, fluid sampling, filtering, degassing, and testing operations using the exemplary LIF system (610) and relative density system (632) are preferably automated using said PLC/computer (628) system integrated with fully automated analytical chemical process systems (650) comprising the LIF sensor system (610) and the relative density sensor system (632).

FIG. 8B is an exemplary LIF spectrogram for the LIF system (610) described under FIG. 8A. As shown in FIG. 8B, the exemplary LIF spectrogram comprises a correlation between fluorescence intensity (660), represented in arbitrary units, versus botanical marker (d-Limonene) concentration (662), and represented as a percent by volume (% v:v). A typical concentration range of a botanical extract was simulated using orange peel d-limonene extract as a chemical marker, P/N limonene 96, available from Florachem Corporation, Jacksonville, Fla., dissolved into a grain derived ethanol, 200 Proof, P/N CDA 12A-1, 200 Proof (denatured with heptane), available from Lab Alley LLC, Austin, Tex. Four (4) Limonene-Ethanol solutions were created comprising 4%, 6%, 8%, and 10% v:v. In addition, a blank solution comprising 100% ethanol (0% d-limonene) was produced. The simulated botanical extract marker samples, including the EtOH blank, were tested using the FloraSPEC system to determine the linearity of the fluorescence response. It was determined that the optimal (maximum) fluorescence response was produced using an excitation light source of 350 nm which produced a fluorescence emission wavelength of 420 nm. The EtOH blank produces no appreciable fluorescence, which is also the case for neat dense phase CO₂. The resulting curve (664) shown in FIG. 8B demonstrates excellent linearity for the concentration range between 0% and 10%, with an R²=0.9914 (666). Furthermore, the equation (666) can be used with PLC/computer system (FIG. 8A, (628)) and LIF system automation (FIG. 8A, (650)) to monitor the operation of the CO₂ SALLE subsystem (FIG. 7, (484)), and particularly the level of d-Limonene present in the CO₂ salted-out solvent mixture (FIG. 7, (602)) during dense phase CO₂ withdrawal, desolvation, and recycling operations to maintain maximum dense phase CO₂ co-extraction efficiency.

FIG. 8C provides an exemplary solvent extraction curve representing an idealized profile for a typical botanical extraction process. As discussed in Ballesteros, L. F. et al., “Selection of the Solvent and Extraction Conditions for Maximum Recovery of Antioxidant Phenolic Compounds from Coffee Silverskin”, Food Bioprocess Technol (2014) 7:1322-1332 (Ballesteros et al.), optimization of extraction conditions is an important aspect of any solid-solvent extraction process. With regards to the present invention, the solvent chemistry, solvent-botanical extract ratios, solvent-botanical solid ratio, extraction time, extract recovery processes, pressure, and temperature represent the key process variables (KPVs), as they affect the kinetics of a particular biomaterial extraction process and the bioactivity of the resulting extracts. Therefore, it is critical to understand and develop optimal extraction and extract recovery conditions. However, the optimization process can be very time consuming and expensive, particularly if the incoming source or supply of solid biomaterial and extract content is highly variable. In this regard, optimizing, monitoring, and controlling extraction and extract recovery conditions under variable conditions can be particularly challenging. Often, extraction times are extended, which causes over-processing that wastes time, energy, and materials. As such, the exemplary LIF system is a cost- and performance-effective alternative to conventional trial-and-error, off-line wet bench instrumental methods, and extended extraction cycles conventionally used to optimize biomaterial extraction methods. Now referring to FIG. 8C, for any given botanical material extraction system (including solvent, solvent blends, biomaterial, dissolved biomaterial extracts, and dissolved bio-based additives) and set of KPVs, there exists an optimal extraction profile which is described by one or more botanical marker concentrations (670) represented as ([C]) over an extraction time (672), represented as (t). Using the exemplary LIF system of the present invention, the solvent-solid-extract system can be monitored in real-time. During a particular botanical extraction process and method of the present invention, one or more botanical chemical markers, for example d-limonene (674), are monitored to produce an optimized extraction profile comprising the initial detection of the botanical marker (676), followed by a noticeable increase in botanical marker concentration (678), and finally to a leveling off of the botanical marker concentration (680), which is an indication of a solvent-extract saturation condition (682). An optimized extraction process would maintain the botanical marker concentration(s) somewhere along the extraction curve (684), representing an optimal extraction rate (d[C]/dt), between the initial botanical extract concentration (628) and the leveling off point (630). Using this scheme, an extraction process can be optimized in terms of extraction solvent flowrate or exchange (i.e., neat liquid CO₂ exchange during the CO₂ salting-out process). For example, the botanical marker concentration level(s) can be monitored to a certain maximum concentration level (686), whereupon the solvent and dissolved extracts are removed (688) from the extraction vessel and fresh solvent is introduced (690) to maintain the optimized extraction rate (d[C]/dt, (684)). Alternatively, fresh extraction solvent can be continuously flowed (692) through the extraction vessel to maintain the optimal extraction rate (d[C]/dt, (684)).

Having described exemplary aspects of the processes and apparatuses of the present invention, following is a more detailed discussion of the CO₂ aerosol generation system, CO₂ aerosol assembly, and use of same in the present invention.

FIG. 9 is a schematic showing an exemplary system for producing and delivering a CO₂ solid-liquid (s→l) near-cryogenic aerosol using a liquid CO₂ capillary condensation process, referred to herein as a CO₂ aerosol assembly. The exemplary CO₂ aerosol assembly of FIG. 9 is used in the present invention to inject, cool, and saturate an extraction solvent system with pure CO₂. In addition, the exemplary CO₂ aerosol assembly of FIG. 9 is also used to crystallize (CO₂ component) and desolvate a CO₂ salted-out solvent mixture containing one or more solvated or desolvated extracts to produce a tincture comprising WSWE compounds and extracts.

Now referring to FIG. 9, the exemplary CO₂ aerosol assembly as used as a cooling device is supplied by a source of liquid CO₂ (700). For example, liquid CO₂ (700) is supplied at a pressure between 600 psi and 1000 psi and a temperature between 10° C. and 30° C. from a high-pressure liquid CO₂ cylinder equipped with a siphon tube or from dense phase CO₂ recycling system. Liquid CO₂ is selectively and controllably injected into a capillary condensing tube segment (702) using a liquid CO₂ valve (704) connected to a manually adjustable micrometering valve (706). Said capillary condensing tube segment (702) comprises one or more open-cycle Joule-Thomson (J-T) expansion capillaries. High pressure capillaries may be flexible (708) or straight (710) tubes with fixed internal diameters or have an expanding diameter (712). Polyetheretherketone (PEEK) polymer tubing (preferred) or stainless-steel capillary tubing may be used.

For example, an exemplary CO₂ aerosol assembly may be constructed using a 0.25-inch internal diameter (I.D.) stainless steel liquid CO₂ supply valve (704), manual or automatic, connected to a 0.25 inch 18-turn high pressure stainless steel micrometering valve (706), which is manually adjusted and set, and which is connected to a section of 0.040 inch I.D. PEEK J-T expansion tube (710). Said exemplary J-T expansion tube (710) has an I.D. of between about 0.002 inches and 0.040 inches and a length of between about 2 inches and 36 inches. One or more J-T expansion tubes (710) may be connected to one liquid CO₂ micrometering valve (706) to provide a range of cooling capacities ranging from approximately 1000 BTU/hour using a 0.010-inch I.D. J-T expansion tube (710) to 5,000 BTU/hour using a 0.040 inch I.D. J-T expansion tube (710). Said exemplary micrometering valve (706) is adjustable from about 0.002 inch (about 1 turn from fully closed) to 0.040 inch (about 18 turns from fully-closed), and is preferably used with one or more J-T expansion tubes having a combined I.D. of 0.040 inch or less.

The CO₂ aerosol assembly thus described produces a micronized, relatively low-pressure CO₂ (solid-gas) aerosol (714). Liquid CO₂ (700) is injected through (opened) valve (704), through preset micrometering valve (706), and into said J-T expansion tube (708, 710, or 712). Following injection into said J-T expansion tube (708, 710, or 712), liquid CO₂ instantly begins to boil, super cool, and condense rapidly within the internal volume and along an internal pressure gradient (high→low) within said J-T expansion tube (708, 710, or 712) to form a mixture of microscopic sublimating solid CO₂ particles and expanding cold CO₂ gas having a temperature of −56.6° C. and a pressure of approximately 5.1 atm. Said microscopic sublimating solid CO₂ aerosol particles possess small crystal diameters, ranging from nanometers to micrometers, possess a surface temperature of −78.5 degree C., and produce a rapid and increasing sublimation pressure once injected into solid-liquid or liquid-liquid extraction system. Solid phase CO₂ particles exhibit a hydrocarbon-like HSP of approximately 22 MPa^{1/2} with a surface energy (S.E.) of approximately 5 mN/m, which enables rapid surface wetting and solvation into organic WSWE compounds such as ethanol (HSP δ_{T}—25.8 MPa^{1/2}, S.E.—21.8 mN/m). Moreover, micronized CO₂ particles have large surface areas and sublimate very quickly following injection. The extraction system remains at approximately ambient pressure during injection and expansion if the system is vented to the atmosphere or increases in pressure during injection if the system vent is closed. This process is called autogenous pressurization or sublimation pressurization.

Finally, the exemplary CO₂ aerosol assembly of FIG. 9 may be used as a desolvation device. A concentrated liquid CO₂-rich CO₂ salted-out solvent mixture (716) containing one or more solvated or desolvated extracts is used in place of pure liquid CO₂. Using the same apparatus and operational scheme described herein for pure liquid CO₂, injection of concentrated liquid CO₂-rich CO₂ salted-out solvent mixture (716) into the CO₂ aerosol assembly and expansion under atmospheric conditions produces a tincture (718) comprising WSWE compounds and dissolved extracts, for example as described under FIG. 7 (582, 590).

Having thus described exemplary and preferred aspects and embodiments of various extraction and desolvation processes and apparatuses of the present invention, following is a discussion of a novel method for producing mixtures of bio-based emulsifiers, including both tinctures and extracts, and emulsions employing same.

FIG. 10A is a schematic describing a novel use of an ozonation process to alter the chemistry of beverage and biomaterial extracts to produce oxygenated tinctures or concentrates for producing bio-based extract-infused emulsions. Referring to FIG. 10A, a CO₂ salted-out solvent mixture (730), for example a tincture containing EtOH and EtOH-soluble compounds such as alcoholic beverage extracts and additives, and a fractional amount of co-extracted water, is placed into a reservoir (732). Following this, said CO₂ salted-out solvent mixture (730) is processed according to the following exemplary method:

Step 1: A method for preparing a bio-based mixture containing one or more oxygenated bio-based emulsifiers, the method comprising:

a. reacting ozonated gas (734), purified air or oxygen, with a decanted and desolvated (i.e., gross CO₂ removed) CO₂ salted-out solvent mixture (730) or tincture containing one or more unsaturated biomaterial and/or alcoholic beverage extracts and additives to form a mixture of unsaturated biomaterial and/or alcoholic beverage extracts and oxygenated extracts and additives, an oxygenated CO₂ salted-out solvent mixture (736); and
b. monitoring and controlling oxygenation level in said oxygenated CO₂ salted-out solvent mixture (736) by light-induced fluorescence spectroscopy, a digital timer, or a viscosity sensor (all not shown);

wherein said oxygenated CO₂ salted-out solvent mixture (736) may be used directly to form bio-based extract infused water-in-oil and oil-in-water emulsions.

The method of Step 1, whereby said oxygenated CO₂ salted-out solvent mixture (736) is distilled (738) to form a purified EtOH liquid (740), which may be recycled back to the originating CO₂ SALLE process, and an oxygenated emulsifier concentrate (742). The method of Step 1, wherein the ozonated gas (734) has a concentration between 0.2 mg/hour and 15000 mg/hour of ozone gas at a temperature between −20 degrees C. and 30 degrees C., and a pressure of about 1 atm.

The method of Step 1, wherein said CO₂ salted-out solvent mixture (730) contains one or more unsaturated natural substances such as cannabinoids, terpenoids, flavonoids, natural oils, bio-based oils and alcohols, garlic oil, lecithin, soybean oil, coconut oil, olive oil, rapeseed oil, corn oil, safflower oil, long-chain alcohol, oleic acid, and oleyl alcohol, among other unsaturated natural and synthetic compounds and mixtures of same, and suitable for use in foods, beverages, pharmaceuticals, cosmetics, and lotions.

The method of Step 1, wherein said CO₂ salted-out solvent mixture (730) is reacted with the ozonated gas in the presence of deionized water and additives to form an oxygenated emulsion. The method of Step 1, wherein said oxygenated CO₂ salted-out solvent mixture (736) is sparged with compressed air, nitrogen or carbon dioxide for a predetermined period of time to remove residual, unreacted ozone gas.

The method of Step 1, wherein oxygenated CO₂ salted-out solvent mixture (736) and oxygenated emulsifier concentrate (742) are used as emulsifying agents during the manufacture of foods, beverages, pharmaceuticals, cosmetics, and lotions. The method of Step 1, wherein a source of concentrated oxygen for said ozonated gas (734), and which is used for ozonation reactions, is derived from a semi-permeable gas membrane. The method of Step 1 wherein the level of oxygenated extractable substance formed in said CO₂ salted-out solvent mixture is controlled using a digital timer or viscosity sensor.

FIG. 10B describes the effect of ozonation of an exemplary plant extract, oleic acid, including changes in chemical and physical properties which enable improved emulsification. Referring to FIG. 10B, an oleic acid molecule (750) containing an unsaturated carbon-carbon double bond (752) is ozonated (754) to form an oxygenated oleic acid molecule (756) now containing an 1,2,4 trioxolane functional group (758). The ozonation process (or molecular “oxygenation” process) can be direct or indirect. Direct oxygenation of an unsaturated bio-based compound involves adding ozone to a water and/or ethanol-based solution containing one or more unsaturated bio-based compounds and additives. Indirect oxygenation involves first adding ozone to a water and/or ethanol-based solution to form an oxygenated solution and then mixing same with one or more unsaturated bio-based compounds and additives. Exemplary ozone generators for practicing the present invention include an air or oxygen fed corona generator and electrolytic generator (in-situ ozone generator).

Using the hydrophilic-lipophilic balance (HLB) Equation 2 (Eq. 2), described under Griffin, W. C., “Calculation of HLB Values of Non-Ionic Surfactants”, Journal of the Society of Cosmetic Chemists, 5 (4), 1954, pp. 249-256 (Griffin HLB Equation), the HLB value for the oleic acid molecule (750) is increased from HLB=2 (760) to HLB=5 (762). The result of ozonation is an oxygenated oleic acid molecule possessing a 150% increase in HLB value favoring the formation of a water-in-oil emulsion, a 17% increase in molecular mass, increased cohesion energy favoring improved water solubility, a larger polar surface area favoring water solubility, and a higher boiling point.

$$Equation 2 ( Eq . 2 ) - Griffin HLB Equation$$

$$HLB = \frac{20 \times M {W}_{O G}}{M {W}_{O}} \\ {MW}_{O G} - Su \text{m:} Molecular Weight of Oxygenated Functional Groups {MW}_{o} - Molecular Weight of Oleic Acid or Oxygenated Oleic Acid$$

Two exemplary bio-based compounds for formulating oxygenated emulsifiers and emulsions using the oxygenation method and process described under FIGS. 10a and 10B include Lecithin, a natural product of plant and animal tissue, and diallyl disulfide, a natural product found abundance in garlic oil extracts. In this regard, the Aulton HLB Scale is described in Aulton, M. E., “Pharmaceutics: The Science of Dosage Form Design” 2nd edition, Churchill Livingstone, 2002. p. 96 (Aulton HLB Scale). The Aulton HLB Scale shows that oil-in-water (O/W) emulsions are favored using emulsifiers with an HLB value range between 8 to 16, and water-in-oil (W/O) emulsions are favored using emulsifiers with an HLB value range between 3 to 6. With respect to Lecithin, a cationic compound, ozonation produces an oxygenated Lecithin molecule with an increase in the HLB value from between 2 to 4 to between 4 to 8 and is dependent upon the ozonation dose rate. Both W/O and O/W emulsions can be formulated using lecithin and oxygenated lecithin mixtures. With respect to diallyl disulfide, ozonation produces an oxygenated disulfide molecule with an increase in the HLB value from 0 (completely lipophilic/hydrophobic molecule) to between HLB=4 to HLB=8 and is dependent upon the ozonation dose rate. Both W/O and O/W emulsions can be formulated using a diallyl disulfide/oxygenated disulfide mixture.

Finally, the present invention discloses two different methods for producing a decarboxylated extractable substance. Cannabis and hemp, among many other botanical products, in their natural or raw states do not provide potent psychoactive or medicinal effects. Achieving these desirable effects requires a process called decarboxylation. The decarboxylation process “activates” chemical compounds in cannabis and hemp so that the human body can use them. Specifically, raw cannabis and hemp and cannabis contain non-psychoactive and synergistic carboxylic acids such as tetrahydrocannabinolic acid (THCA), cannabidiolic acid (CBDA), and cannabigerolic acid (CBGA). When heated, these carboxylic acids transform (over a period of time) to cannabinoids: (psychoactive) tetrahydrocannabinol (THC), (synergistic) cannabidiol (CBD), and (synergistic) cannabigerol (CBG), all with the loss of a CO₂ molecule. These exemplary cannabinoids interact with the body's endocannabinoid system vis-à-vis a psychoactive or non-psychoactive mode.

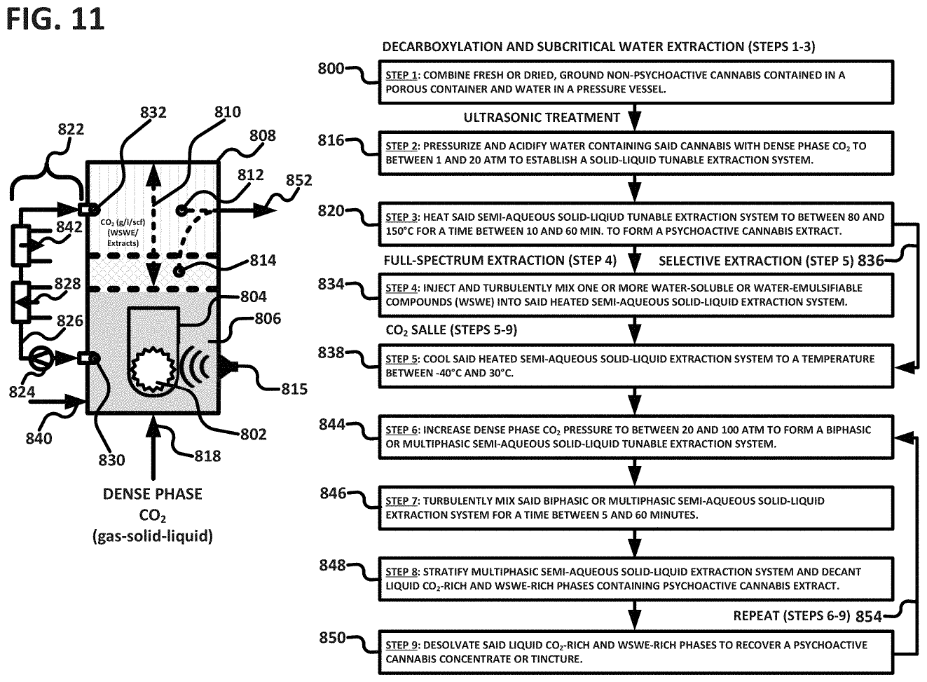

A first decarboxylation method comprises a straightforward thermal procedure whereby a desolvated CO₂ salted-out solvent mixture containing extractable substance produced from a plant material is heated to a temperature between 100° C. and 120° C. for a time between 30 minutes and 120 minutes. This method is simple and useful particularly if the WSWE compound containing the extractable substance has a high boiling point, for example a vegetable oil. However, loss of volatile phytochemicals will occur during this process if performed in a system which is open to the atmosphere. A second decarboxylation process is disclosed which performs the decarboxylation process in-situ and within a closed system during a heated water-based extraction process followed by a CO₂ SALLE process, and is discussed in detail under FIG. 11.

FIG. 11 provides a schematic and flowchart describing an exemplary hybrid cannabis (or hemp) decarboxylation and extraction process utilizing a semi-aqueous extractant, employing subcritical water temperature and pressure conditions, and followed by a CO₂ SALLE process. The decarboxylation-extraction process of FIG. 11 is derived from the exemplary subcritical water-CO₂ SALLE processes described under CO₂ SALLE Method II described under FIG. 5A and modified or hybrid subcritical water-CO₂ SALLE processes described under FIG. 6A, FIG. 6B, and FIG. 7.

Compared to conventional decarboxylation processes, the hybrid decarboxylation-extraction process of the present invention provides several operational advantages and distinctions including: 1) eliminating the need for a separate thermal decarboxylation process, 2) elimination of volatile extract losses, 3) elimination of offgassing and outgassing odors common to heated air thermal treatment schemes, and 4) a carbonic acid-catalyzed decarboxylation process. The hybrid decarboxylation-extraction process can be used to process any variety of cannabis and hemp, or any natural product.

Now referring to FIG. 11, following is a decarboxylation-extraction method and process using an exemplary non-psychoactive cannabis, containing a significant amount of THCA, immersed in a semi-aqueous solution, decarboxylated under a dense phase CO₂ gas atmosphere under subcritical water temperature conditions, and co-extracted using a dense phase CO₂ liquid. The exemplary decarboxylation-extraction method described under FIG. 11 provides an exhaustive extraction process to produce a full-spectrum psychoactive cannabis and hemp extracts.

The exemplary decarboxylation-extraction method comprises the following steps:

Step 1 (800): Combining fresh or dried, and ground non-psychoactive cannabis (802), contained in a removable porous container (804) such as a glass thimble, perforated basket, porous fabric, or centrifuge drum, and water (806) in a pressure vessel (808). Cannabis (802) varieties include for example Cannabis sativa, cannabis indica, or Cannabis ruderalis. The exemplary cannabis plant contains numerous phytochemicals, including non-psychoactive and synergistic carboxylic acids such as THCA, CBDA, and CBGA. In this example application of the decarboxylation-extraction method, the cannabis plant used contains a significant amount of THCA content to be decarboxylated to the psychoactive THC. The volume (and level) of water (806) and quantity of ground cannabis (802) contained in said pressure vessel (808) is controlled using a level sensor (not shown) to provide an internal freeboard space (810) that allows for the formation of a predetermined volume of liquid CO₂-rich CO₂ salted-out solvent phase and mixture (812) and (optionally) a WSWE-rich CO₂ salted-out solvent phase and mixture (814) during CO₂ SALLE operations. Moreover, said cannabis (802) immersed in water (807) is pretreated for a predetermined amount of time using 20 kHz or 40 kHz ultrasonic horn (815) possessing sufficient power to disrupt cellular structures contained in said cannabis (802) and to provide significant preheating of the water (807).

Step 2 (816): Said pressure vessel (808) is sealed, following which the mixture of (ultrasonically treated) cannabis (802) and water (806) is pressurized with dense phase CO₂ (818) to acidify and provide an internal CO₂ vapor pressure between 1 atm and 20 atm, establishing a tunable semi-aqueous solid-liquid extraction system comprising cannabis, water, and aqueous CO₂. An internal pressure sensor and external CO₂ pump (both not shown) preferably control the internal pressure of said pressure vessel (808).

Step 3 (820): Heating said tunable semi-aqueous solid-liquid extraction system to a decarboxylation temperature between 80° C. and 150° C., with CO₂ acidification and CO₂ vapor pressure of between 1 atm and 20 atm, for example, and held for a predetermined carbonic acid-catalyzed thermal decarboxylation time between 10 and 60 minutes. A conventional decarboxylation temperature and time schedule is based on known reaction conditions and rates to convert non-psychoactive extractable carboxylic acids to their neutral forms; for example, tetrahydrocannabinolic acid (THCA) to (psychoactive) tetrahydrocannabinol (THC), cannabidiolic acid (CBDA) to (synergistic) cannabidiol (CBD), and cannabigerolic acid (CBGA) to (synergistic) cannabigerol (CBG). For example, Perrotin-Brunel, H. et al., “Decarboxylation of Δ⁹-tetrahydrocannabinol: Kinetics and Molecular Modeling”, Journal of Molecular Structure 987 (2011) 67-73 (Perrotin-Brunel et al.), FIG. 2 and Table 1 (Page 69), provide reaction plots and rates for the THCA→THC decarboxylation process between 90° C. and 140° C., and which are used in this Step 3 (820). Further to this, Perrotin-Brunel et al. modelled weak and strong organic and inorganic acid-catalyzed decarboxylation rates, and determined that acidification enhanced decarboxylation rates through protonated keto-enol reaction pathways. As such, it is reasonable to suggest that combining carbonic acid (produced under high aqueous CO₂ pressure) with a conventional thermal treatment schedule under Step 3 (820) represents a process intensified method for both cannabis decarboxylation and subcritical water extraction through protonation (CO₂ acidification) and heat energy mechanisms. Heat may be added to the semi-aqueous solid-liquid extraction system, for example, using a reversible solid-liquid mixing loop (822) comprising a recirculating pump (824), recirculating pipe (826), and fluid heating means (828). Fluid heating means (828) includes any conventional technique such as an electric fluid heater or steam heat exchanger. The solid-liquid mixing loop fluidly interconnects the lower interior hemisphere (830) of the pressure vessel (808) to the upper interior hemisphere (832) of the pressure vessel (808). Using this reversible fluid mixing scheme, semi-aqueous solution and dense phase CO₂ can be flowed from the lower hemisphere to the upper hemisphere, and vis-a-versa, respectively. An internal temperature sensor (not shown) is preferably used in combination with said reversible solid-liquid mixing loop.

Following Step 3 (820), said heated semi-aqueous solid-liquid extraction system containing decarboxylated and subcritical water extracted cannabis may be mixed with a WSWE compound or mixture under Step 4 (834) and processed sequentially under Steps 5-9 to produce a full-spectrum cannabis concentrate or tincture. Alternatively, Step 4 (834) may be skipped (836) and said heated semi-aqueous solid-liquid extraction system containing decarboxylated and subcritical water extracted cannabis may be cooled directly under Step 5 (838). Not adding a WSWE compound or mixture at this stage provides selectivity in the exemplary decarboxylation and extraction process. For example, absent a polar miscible WSWE compound, nonpolar liquid CO₂ will selectively and predominantly extract nonpolar terpenoids and cannabinoids from the cooled semi-aqueous solid-liquid extraction system during subsequent CO₂ SALLE concentration and recovery Steps 5-9.

Step 4 (834): Injecting and mixing (840) one or more water-soluble or water-emulsifiable compounds, and optional additives, into said heated semi-aqueous solid-liquid extraction system containing decarboxylated (psychoactive) and subcritical water extracted cannabis.

Step 5 (838): Cooling said heated semi-aqueous solid-liquid extraction system containing decarboxylated and subcritical water extracted cannabis, and WSWE/additive compounds, to a temperature between −40° C. and 30° C. Heat may be removed from the heated semi-aqueous solid-liquid extraction system using the exemplary solid-liquid mixing loop (822) used for heating, and comprising a recirculating pump (824), recirculating pipe (826), and fluid cooling means (842). Fluid cooling means (842) include any conventional technique, for example a chilled water heat exchanger, and which may be used with near-cryogenic CO₂ aerosol injection. For example, following a pre-cooling stage using a chilled water heat exchanger to below 100° C., a vent valve (not shown) connected to the pressure vessel (808) may be opened to the atmosphere, whereupon a near-cryogenic dense phase CO₂ (s→g) aerosol may be injected into the pre-cooled semi-aqueous solid-liquid extraction system through dense phase CO₂ inlet (818) to further cool and saturate the solid-liquid solvent system with aqueous CO₂. An internal temperature sensor (not shown) is preferably used in combination with said solid-liquid mixing loop, fluid cooling means (842), and CO₂ aerosol injection (818).

Step 6 (844): Increasing dense phase CO₂ pressure through dense phase CO₂ inlet (818) to between 20 and 100 atm at a temperature between −40° C. and 30° C. to form a biphasic or multiphasic (if WSWE compounds are present) semi-aqueous solid-liquid extraction system. An internal pressure sensor and external CO₂ pump (both not shown) preferably control the internal pressure of said pressure vessel (808).

Step 7 (846): Turbulently mixing said biphasic or multiphasic semi-aqueous solid-liquid extraction system using said mixing loop (622) for a predetermined time between 5 and 60 minutes to facilitate the extraction of decarboxylated cannabinoids and other extractables from cannabis (802). Turbulent mixing may be accomplished using the exemplary solid-liquid mixing loop (822), previously described, and preferably flowing liquid CO₂-rich and WSWE-rich CO₂ salted-out solvent mixtures or phases from the upper hemisphere (832) into the lower hemisphere (830). Alternative mixing means include ultrasonics, mechanical blade, and centrifuge drum.

Step 8 (848): Halting mixing to allow the biphasic or multiphasic semi-aqueous solid-liquid extraction system to stratify into distinct layers; a water-rich semi-aqueous phase (806), WSWE-rich CO₂ salted-out solvent mixture or phase (814), and a liquid CO₂-rich CO₂ salted-out solvent mixture or phase (812) containing a portion of cannabis extracts. Following this, the CO₂ salted-out solvent mixtures (i.e., liquid CO₂-rich (812) and WSWE-rich (814) phases) containing decarboxylated (psychoactive) cannabis extracts are decanted (852) from said pressure vessel (808).

Step 9 (850): Desolvating said CO₂ salted-out solvent mixtures (i.e., liquid CO₂-rich (812) and WSWE-rich (814) phases) containing decarboxylated (psychoactive) cannabis extracts to concentrate and recover said psychoactive cannabis extracts as a concentrate or tincture, as described previously herein. Following this, dense phase CO₂ pressurization, mixing, extraction, stratification, decanting, and desolvating Steps 6-9 may be repeated (854) as needed to recover cannabis extracts from the semi-aqueous solid-liquid solvent system.

The exemplary decarboxylation-extraction process described under Steps 1-9 may use dense phase CO₂ under supercritical conditions, which provides additional selectivity during co-extraction and desolvation operations. Moreover, higher semi-aqueous solid-liquid extraction system temperatures may be used to enhance extraction efficiency. As such, the entire pressure-temperature operating window for providing pressurization, heating, and cooling processes during a decarboxylation-extraction process is a dense phase CO₂ pressure between 1 atm and 340 atm and a semi-aqueous solid-liquid extraction system temperature between −40° C. and 300° C. More preferably, said dense phase CO₂ is contacted with said semi-aqueous solid-liquid extraction system at a temperature between −20° C. and 150° C. and at a pressure between 1 atm and 150 atm. In addition, process intensification techniques such as microwave pre-treatment, ultrasonic processing, and centrifugation may be employed to optimize the decarboxylation-extraction process conditions and extract yields.

Still moreover, the decarboxylation-extraction process described under FIG. 11 can be performed in a sequential order using two pressure vessel systems equipped with quick-opening closures: a first hot treatment pressure vessel system equipped with heating and mixing means, including an ultrasonic horn, is used to perform Steps 1-3 and a second cold treatment pressure vessel system equipped with cooling and mixing means is used to perform Steps 4-9. Following Steps 1-3 performed in said first hot treatment pressure vessel system, the semi-aqueous extractant is removed, cooled, and transferred to said second cold treatment pressure vessel system, and processed using a CO₂ SALLE method, for example as described under FIG. 7.

Following this, the processed semi-aqueous extractant (referred to as “Raffinate”) is recycled or discharged to drain. The decarboxylated cannabis contained in said hot treatment pressure vessel system is removed and transferred to said second cold treatment pressure vessel system and processed using a CO₂ SALLE method to extract and recover residual nonpolar compounds. Following this, exhaustively extracted and decarboxylated cannabis (referred to as “Marc”) is removed from the second cold treatment pressure vessel system and disposed of or recycled.

Finally, the exemplary decarboxylation-extraction process described under FIG. 11 can be performed using an alcoholic beverage to replace water under Step 1 (800) or used as the WSWE additive under Step 4 (834). As previously discussed herein, alcoholic beverages contain natural WSWE compounds such as fermented ethanol and ethanol-soluble organic compounds. Moreover, high Proof alcoholic beverages such as Vodka or Grain Alcohol reduce subcritical water extraction temperatures required to achieve organic solvent-like solubility chemistry while achieving required decarboxylation temperature and time. Alcoholic beverages used in combination with botanical mixtures, for example herbs, spices, hemp, and psychoactive plants such as cannabis indica, Cannabis sativa, or Cannabis ruderalis, and processed using the CO₂ SALLE multiphasic extraction process produce natural, flavorful, and psychoactive extract concentrates or tinctures.

Having described exemplary aspects of the present invention, and its usefulness for extracting beneficial compounds from a biomaterial, it can be understood that the present invention can be used in many other novel solid-liquid and liquid-liquid extraction applications. In this regard, Table 5 provides examples of use for the present invention.

TABLE 5
Examples of Use

| | Method | Description |
|---|---|---|
| 1. | CO₂ Salting-out Assisted Liquid-Liquid Extraction (CO₂ | Dense phase CO₂ (gas, liquid, or supercritical) is used to selectively |
| | SALLE) Method | expand and salt-out one or more water-soluble or water- |
| | | emulsifiable compounds containing one or more extractable |
| | | substances from a semi-aqueous solution used in a solid-liquid or |
| | | liquid-liquid extraction process. |
| 2. | A Method for Natural Products Extraction | A plant material is extracted using a mixture of dense phase CO₂ |
| | | and a semi-aqueous phase containing one or more water-soluble or |
| | | water-emulsifiable substances to extract, concentrate, and recover |
| | | full-spectrum natural extracts including cannabinoids, terpenes, |
| | | flavonoids, and carotenoids. |
| 3. | A Method for Food and Beverage Infusion | An alcoholic beverage containing fermented ethanol and ethanol- |
| | | soluble compounds such as natural beverage flavors is expanded |
| | | and salted-out, dissolved into a dense phase CO₂, and (optionally) |
| | | used to co-extract one or more herbs or spices to form a natural, |
| | | healthful, and flavorful infusion. |
| 4. | A Process for Desolvating a Liquid or Solid Extract from | A near-cryogenic solid-gas spray method of the present invention |
| | Dense Phase CO₂ | for desolvating liquid and solid extracts from dense phase CO₂. |
| 5. | A Method for Extracting Organic Compounds from a | A fermentation broth is extracted using the present invention to |
| | Fermented Broth | recover dissolved organics for medical and pharmaceutical uses. |
| 6. | A Method for Forming Water-Oil and Oil-Water | A botanical extract mixture is partially oxygenated to form a water- |
| | Botanical Emulsions | oil or oil-water emulsifying agent for formulating emulsions |
| | | containing same. |
| 7. | Method and Apparatus for a Hybrid Water-CO₂ | A water-based extraction process is used as a primary extraction |
| | Extraction Process | process in combination with a dense phase CO₂ co-extraction |
| | | process and followed by a CO₂ SALLE process of the present |
| | | invention to exhaustively or selectively extract a liquid or solid |
| | | substance. |
| 8. | A Method for Alcohol Recovery from Aqueous Solutions | Recovery of alcohol such as isopropanol and ethanol from an |
| | | industrial wastewater or fermented liquid. |
| 9. | A Method for Environmental Sample Analysis | Environmental samples such as plants, soils, animal tissues, and |
| | | waters are extracted to recover pollutants such as dissolved or |
| | | suspended oils, chelated metals, pesticides, and pharmaceutical |
| | | drugs for in-situ or ex-situ instrumental analysis. |
| 10. | A Method for Concentrating and Analyzing an Extract | Extracts produced by a solid-liquid or liquid-liquid extraction |
| | from a Solid-Liquid or Liquid-Liquid Extraction Process | process are concentrated and analyzed using as analytical chemical |
| | | process. |
| 11. | A Method for Decarboxylating and Extracting Cannabis | Fresh or dried cannabis is decarboxylated and extracted using |
| | using Subcritical Water and CO₂ | subcritical water and a multiphasic CO₂ SALLE process. |

The present invention is useful for extracting, concentrating, and recovering one or more organic, inorganic, and ionic compounds from a liquid or solid substance. Said organic, inorganic, or ionic compounds may be useful, for example, as food, beverage, nutraceutical, pharmaceutical, or cosmetic additives. Said organic, inorganic, or ionic compounds may be useful, for example, as analytes in an environmental pollution assessment. Said liquid substances may be, for example, potable waters, water-based extractants, or industrial wastewaters. Said solid substances may be, for example, plants, vegetables, fruits, animal tissue, and contaminated soils.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the title, headings, terms, and phrases used herein are not intended to limit the subject matter or scope; but rather, to provide an understandable description of the invention. The invention is composed of several sub-parts that serve a portion of the total functionality of the invention independently and contribute to system level functionality when combined with other parts of the invention. The terms “CO2” and “CO₂” and carbon dioxide are interchangeable. The terms “natural product” and “natural substance” and “biomaterial” and “plant-based” and “botanical products” are interchangeable. The terms “bio-based” and “natural” are interchangeable. The terms “Hansen Solubility Parameter” and “HSP” and “solubility parameter” and “cohesion energy” and the symbol “δ” are interchangeable. The terms “extract” and “extractable substance” and “extractable material” and “extractable compound” and “analyte” are interchangeable. The terms “extraction vessel” and “pressure vessel” and “process vessel” and “extractor” are interchangeable. The term “CO₂ SALLE” includes both CO₂ salting-out and CO₂ solvent expansion phenomena assisted liquid-liquid extraction. The terms “a” or “an”, as used herein, are defined as one or more than one. The term plurality, as used herein, is defined as two or more than two. The term another, as used herein, is defined as at least a second or more. The terms including and/or having, as used herein, are defined as comprising (i.e., open language). The term coupled, as used herein, is defined as connected, although not necessarily directly, and not necessarily mechanically. Any element in a claim that does not explicitly state “means for” performing a specific function, or “step for” performing a specific function, is not to be interpreted as a “means” or “step” clause as specified in 35 U.S.C. Sec. 112, Parag. 6. In particular, the use of “step of” in the claims herein is not intended to invoke the provisions of 35 U.S.C. Sec. 112, Parag. 6.

Incorporation of Reference: All research papers, publications, patents, and patent applications mentioned in this specification are herein incorporated by reference to the same extent as if each individual publication, patent, or patent appl. was specifically and individually indicated to be incorporated by reference.

We claim:

1. A semi-aqueous extraction method for recovering an extract from a substance, the steps comprising:
a. Placing the substance into a pressure vessel;
b. Adding a semi-aqueous solution, comprising a mixture of water and water-soluble or water-emulsifiable compound, to the pressure vessel;
c. Pressurizing said semi-aqueous solution and the substance using dense phase CO₂ to establish a tunable extraction system in the pressure vessel;
d. Expanding and salting-out said tunable extraction system using said dense phase CO₂ to produce a first separated phase, which comprises the water-soluble or water-emulsifiable compound containing the extract; and
e. Simultaneously co-extracting said first separated phase into said dense phase CO₂ to produce a second separated phase, which comprises a CO₂ salted-out solvent mixture containing the extract.

2. The semi-aqueous extraction method of claim 1, wherein said substance comprises natural product, pomace, animal tissue, soil, sludge, slurry, potable water, alcoholic beverage, fermentation broth, industrial wastewater, fermented food, or water-based extractant.

3. The semi-aqueous extraction method of claim 1, wherein said extract comprises phytochemical, essential oil, polyphenol, fermented compound, fermented ethanol, ethanol-soluble compound, decarboxylated compound, psychoactive compound, terpenoid, cannabinoid, flavonoid, carboxylic acid, protein, oxygenated compound, organic compound, metalorganic compound, inorganic compound, chemical pollutant, or ionic compound.

4. The semi-aqueous extraction method of claim 1, wherein said water-soluble or water-emulsifiable compound comprises alcohol, polyol, ketone, ester, nitrile, ether, organosulfur compound, surfactant, emulsion, hydrotrope, or aqueous carbon dioxide.

5. The semi-aqueous extraction method of claim 1, wherein said dense phase CO₂ comprises gaseous CO₂, solid CO₂, liquid CO₂, or supercritical CO₂.

6. The semi-aqueous extraction method of claim 1, wherein said dense phase CO₂ is contacted with said tunable extraction system at a temperature between −40° C. and 300° C. and at a pressure between 1 atm and 340 atm.

7. The semi-aqueous extraction method of claim 1, wherein said dense phase CO₂ is preferably contacted with said tunable extraction system at a temperature between −20° C. and 150° C. and a pressure between 5 atm and 150 atm.

8. The semi-aqueous extraction method of claim 1, wherein said CO₂ salted-out solvent mixture comprises gaseous CO₂ and CO₂ expanded and salted-out water-soluble or water-emulsifiable compound;
said CO₂ salted-out solvent mixture comprises liquid CO₂ and CO₂ expanded and salted-out water-soluble or water-emulsifiable compound; or
said CO₂ salted-out solvent mixture comprises supercritical CO₂ and CO₂ expanded and salted-out water-soluble or water-emulsifiable compound.

9. The semi-aqueous extraction method of claim 1, wherein said CO₂ salted-out solvent mixture is a water-soluble or water-emulsifiable-rich CO₂ salted-out solvent mixture containing the extract and a dense phase CO₂-rich CO₂ salted-out solvent mixture containing the extract.

10. The semi-aqueous extraction method of claim 1, wherein a quantity of said water-soluble or water-emulsifiable compound contained in said tunable extraction system and Hansen Solubility Parameters of said water-soluble or water-emulsifiable compound contained in said tunable extraction system are calculated based on an amount of the extract to be extracted by said water-soluble or water-emulsifiable compound and Hansen Solubility Parameters of the extract to be extracted by said water-soluble or water-emulsifiable compound.

11. The semi-aqueous extraction method of claim 1, wherein a quantity of said dense phase CO₂ and Hansen Solubility Parameters of said dense phase CO₂ are calculated based on an amount of said water-soluble or water-emulsifiable compound containing the extract to be co-extracted by said dense phase CO₂ and Hansen Solubility Parameters of said water-soluble or water-emulsifiable compound containing the extract to be co-extracted by said dense phase CO₂.

12. The semi-aqueous extraction method of claim 1, wherein said tunable extraction system is mixed with additives comprising purified water, organic acid, organic salt, inorganic salt, surfactant, co-surfactant, enzyme, pH buffer, chelation agent, triacetin, or ozone.

13. The semi-aqueous extraction method of claim 1, wherein said water-soluble or water-emulsifiable compound contained in said tunable extraction system is selectively expanded and salted-out using CO₂ pressure, CO₂ temperature or CO₂ volume.

14. The semi-aqueous extraction method of claim 1, wherein a concentration of said water-soluble or water-emulsifiable compound in said tunable extraction system or said CO₂ salted-out solvent mixture is between 0.1% and 95% by volume.

15. The semi-aqueous extraction method of claim 1, wherein said CO₂ salted-out solvent mixture is used in a secondary process comprising solid-liquid extraction process, liquid-liquid extraction process, analytical chemical process, desolvation process, ozonation process, fractionation process, or decarboxylation process.

16. The semi-aqueous extraction method of claim 15, wherein said desolvation process comprises utilizing gravity separation, phase separation, near-cryogenic phase separation, high pressure distillation, atmospheric distillation, vacuum distillation, membrane separation, gas flotation, or evaporation to form a desolvated CO₂ salted-out solvent mixture, which comprises a water-soluble or water-emulsifiable compound containing the extract.

17. The semi-aqueous extraction method of claim 16, wherein an ozonated gas is bubbled through said desolvated CO₂ salted-out solvent mixture to form an oxygenated extract.

18. The semi-aqueous extraction method of claim 17, wherein said ozonated gas has a concentration between 0.2 mg/hour and 15000 mg/hour of ozone gas at a temperature between minus 20 degrees C. and 30 degrees C., and a pressure of about 1 atm.

19. The semi-aqueous extraction method of claim 17, wherein the concentration of said oxygenated extract is monitored and controlled using a digital timer or a viscosity sensor.

20. The semi-aqueous extraction method of claim 15, wherein said analytical chemical process comprises analyzing the extract dissolved in said CO₂ salted-out solvent mixture using UV-VIS spectrophotometry, fluorescence spectroscopy, Raman spectroscopy, gas chromatography, high-performance liquid chromatography, ion chromatography, liquid density analysis, or gravimetric analysis.

21. The semi-aqueous extraction method of claim 20, wherein said analytical chemical process is performed in-situ or ex-situ.

22. A semi-aqueous extraction method for recovering an extract from a natural product, the steps comprising:
a. Placing the natural product containing the extract into a first pressure vessel;
b. Adding a semi-aqueous solution, which comprises water and a water-soluble or water-emulsifiable compound, to the first pressure vessel;
c. Pressurizing said semi-aqueous solution and natural product with dense phase CO₂ to a pressure between 1 atm and 340 atm to establish a tunable extraction system within the first pressure vessel;
d. Heating said tunable extraction system contained within the first pressure vessel to a temperature between 30° C. and 300° C. and maintaining temperature for a time between 5 minutes and 120 minutes to produce a heated water-based extractant containing water-soluble or water-emulsifiable compound and extract within the first pressure vessel;
e. Cooling said heated water-based extractant to a temperature between −40° C. and 40° C. during transfer to a second pressure vessel;
f. Expanding and salting-out said cooled water-based extractant within the second pressure vessel using dense phase CO₂ to produce a first separated phase, which comprises water-soluble or water-emulsifiable compound containing the extract;
g. Simultaneously co-extracting said first separated phase in the second pressure vessel with said dense phase CO₂ to produce a second separated phase, which comprises a CO₂ salted-out solvent mixture containing the extract;
h. Transferring said CO₂ salted-out solvent mixture containing the extract to a third pressure vessel; and
i. Desolvating said CO₂ salted-out solvent mixture within the third pressure vessel to concentrate and recover said extract.

23. The semi-aqueous extraction method of claim 22, wherein said natural product comprises plant, vegetable, fruit, nut, spice, herb, hops, root, bark, hemp, or cannabis.

24. The semi-aqueous extraction method of claim 22, wherein said extract is decarboxylated.

25. A semi-aqueous extraction method for forming an alcoholic mixture, the steps comprising:
a. Placing a natural product containing an extract into a pressure vessel;
b. Adding an alcoholic beverage containing fermented ethanol and ethanol-soluble fermented compounds to the pressure vessel;
c. Pressurizing said alcoholic beverage and the natural product using dense phase CO₂ to establish a tunable extraction system in the pressure vessel;
d. Expanding and salting-out said tunable extraction system using said dense phase CO₂ to produce a first separated phase, which comprises fermented ethanol, ethanol-soluble fermented compounds, and the extract;
e. Simultaneously co-extracting said first separated phase using said dense phase CO₂ to produce a second separated phase, which comprises a CO₂ salted-out solvent mixture containing the fermented ethanol, the ethanol-soluble fermented compounds, and the extract; and
f. Desolvating said CO₂ salted-out solvent mixture to concentrate and to form the alcoholic mixture.

26. The semi-aqueous extraction method of claim 25, wherein said alcoholic beverage comprises beer, vodka, port, rum, gin, whiskey, bourbon, brandy, grain alcohol, cognac, tequila, wine, baijiu, sake, soju, hard seltzer, or hard cider.

27. The semi-aqueous extraction method of claim 25, wherein said alcoholic mixture is desolvated to form a non-alcoholic concentrate.
